(19) United States
(12) Patent Application Publication
(10) Pub. No.: US 2025/0283052 A1
(43) Pub. Date: Sep. 11, 2025

(54) THERAPEUTIC NK CELL POPULATIONS

(71) Applicant: Gamida Cell Ltd., Ramat Gan (IL)

(72) Inventors: Tracey LODIE, Jerusalem (IL); Yona GEFFEN, Jerusalem (IL); Aviad PATO, Jerusalem (IL); Julia RIFMAN, Jerusalem (IL); Sherri COHEN, Jerusalem (IL)

(21) Appl. No.: 18/580,407

(22) PCT Filed: Jul. 18, 2022
(86) PCT No.: PCT/US2022/037462

§ 371 Date: Jan. 18, 2024

Related U.S. Application Data
(60) Provisional application No. 63/223,024, filed on Jul. 18, 2021.

(51) Int. Cl. C12N 5/00; A61K 31/675; A61K 31/7076; A61K 35/17; A61K 38/20; A61K 40/15; A61K 40/42; A61P 35/00; C07K 16/30; C12N 5/0783

(52) U.S. Cl. CPC C12N 5/525; A61K 31/675; A61K 31/7076; A61K 35/17; A61K 38/2013; A61K 40/15; A61K 40/42; A61P 35/00; C07K 16/30; C12N 5/0646; A61K 2239/38; C12N 2500/30; C12N 2501/2315

(57) ABSTRACT

Methods of expanding and cryopreserving a natural killer (NK) cell fraction for clinical use are provided, and particularly, methods for providing expanded, cryopreserved NK cell fractions and protocols for their use, which can be employed for applications in cell transplants and infusions for treatment of cancer and other disease.

### RELATED APPLICATIONS

This application is a U.S. National Phase application, filed under U.S.C. § 371, of International Application No. PCT/US2022/037462, filed Jul. 18, 2022, which claims priority to, and the benefit of, U.S. Provisional Application No. 63/223,024, filed Jul. 18, 2021, the contents of each of which are incorporated herein by reference in their entireties.

### FIELD AND BACKGROUND

Infusion of Natural Killer (NK) cells represent an attractive approach to immunotherapeutic treatment of patients with cancers, including hematologic malignancies. However, efficacy has been limited by the short-term persistence of NK cells and their impaired effector function after infusion. The insufficient numbers of NK cells in peripheral blood mononuclear cells and the limited ability of adoptively transferred NK cells to proliferate in vivo, as well as to home to, and be retained in the tumor micro-environment, likely play a role in their limited efficacy to date. Accordingly, there is a need in the art for methods directed to culturing and expanding NK cells such that the resulting NK cell fractions display increased homing, retention and proliferation activities upon in vivo infusion, while maintaining their killing activity.

The present invention relates to methods of culturing natural killer (NK) cells, selection of expanded NK cell populations for administration to subjects in need thereof and the therapeutic use of suitable, ex-vivo expanded NK cell fractions for transplantation in the clinical setting, for treatment of hematological malignancies and other (e.g. malignant) conditions. The present invention also envisions compositions and kits comprising the expanded NK cell fractions.

### SUMMARY

The present disclosure provides NK cell fractions comprising a population of nucleated cells, wherein the population comprises at least 1.0×10⁶ nucleated cells, wherein at least about 70% of the cells in the population are viable, wherein: at least about 70% of the cells in the population are CD56+; no more than about 0.5% of the cells in the population are CD3+; no more than about 10% of the cells in the population are CD19+; no more than about 10% of the cells in the population are CD14+; at least about 44% of the cells in the population are CD49a+; no more than about 27% of the cells in the population are LAG3+; no more than about 32% of the cells in the population are CD200R+; no more than about 25% of the cells in the population are CD57+; at least about 10% of the cells in the population are CD16+; and at least about 10% of the cells in the population are CD62L+.

In some aspects, at least about 90% of the cells in the population are CD56+.

In some aspects, about 90 to about 95% of the cells in the population are CD56+.

In some aspects, about 0.2 to about 0.3% of the cells in the population are CD3+.

In some aspects, at least about 70% of the cells in the population are CD56+/CD3− and no more than about 0.5% of the cells in the population are CD56−/CD3+.

In some aspects, at least about 99% of the cells in the population are CD56+/CD3−.

In some aspects, at least about 15% of the cells in the population are CD62L+.

In some aspects, about 18% to about 70% of the cells in the population are CD62L+.

In some aspects, at least about 20% of the cells in the population are CD16+.

In some aspects, about 20% to about 60% of the cells in the population are CD16+.

In some aspects, no more than about 1% of the cells in the population are LAG3+, no more than about 1.5% of the cells in the population are CD200R+, and no more than about 2.5% of the cells in the population are CD57+.

In some aspects, no more than about 3% of the cells in the population are CD56+/LAG3+, no more than about 11% of the cells in the population are CD56+/CD200R+, and no more than about 4% of the cells in the population are CD56+/CD57+.

In some aspects, at least about 43% of the cells in the population are CD56+/CD16+.

In some aspects, at least about 57% of the cells in the population are CD56+/CD16+.

In some aspects, at least about 78% of the cells in the population are CD56+/CD62L+.

In some aspects, no more than about 77% of the cells in the population are NKp80+. In some aspects, no more than about 15.77% of cells in the population are NKp80+.

In some aspects, the NK cell fraction comprises: i) at least about 17.5×10⁸ nucleated cells; ii) at least about 35×10⁸ nucleated cells; iii) at least about 2.5×10⁹ nucleated cells; iv) at least about 5×10⁹ nucleated cells; v) at least about 1.25×10⁷ nucleated cells; vi) at least about 2.5×10⁷ nucleated cells; vii) at least about 5×10⁷ nucleated cells; or viii) at least about 1×10⁸ nucleated cells.

In some aspects, an NK cell fraction had been previously frozen and subsequently thawed.

The present disclosure provides cryopreserved NK cell fractions comprising an NK cell fraction of the present disclosure and DMSO, wherein the concentration of DMSO is about 10% v/v.

In some aspects, a cryopreserved NK cell fraction is stable: i) at about −80° C. for at least about six weeks; and/or ii) at about −150° C. for at least about 12 months.

The present disclosure provides methods of preparing NK cell fractions, the method comprising: (a) obtaining an apheresis product comprising NK cells and CD3+ cells allogeneic to subject; (b) separating the apheresis product into a CD3-depleted cell fraction and a CD3+ cell fraction; (c) inactivating cells of said CD3+ cell fraction by irradiation; (d) ex vivo culturing said CD3-depleted cell fraction with the inactivated, irradiated CD3+ cell fraction under conditions allowing for cell proliferation, wherein said conditions comprise providing nutrients, serum, IL-15, a CD3 agonist and nicotinamide in an amount between 1.0 mM to 10 mM; (e) supplementing said combined CD3-depleted and CD3+ cell fractions with fresh nutrients, serum, IL-15 and nicotinamide 6-10 days following step (d) to produce an expanded CD3-depleted cell fraction; (f) harvesting said combined CD3-depleted and CD3+ cell fractions 14-16 days following step (d); and (g) washing and concentrating said combined CD3-depleted and CD3+ cell fraction of step (f), thereby producing the NK cell fraction.

In some aspects, an apheresis product is a human apheresis product comprising human NK and CD3+ cell fractions.

In some aspects, a CD3 agonist is OKT3.

In some aspects, said conditions for allowing for cell proliferation comprise: i) human serum at a concentration of about 10% (v/v); ii) IL-15 at a concentration of about 20 ng/ml; iii) OKT3 at a concentration of about 1 μg/ml; iv) nicotinamide at a concentration of about 7.0 mM; and v) nutrients comprising minimal essential cell culture medium.

In some aspects, said CD3-depleted and said irradiated CD3+ cells are seeded at a 1:1 ratio in step (d).

In some aspects, said culturing of step (d) is affected in flasks at i) 0.30-0.40×10⁶ CD3-depleted and 0.30-0.40×10⁶ irradiated CD3+ cells/ml; ii) 0.35×10⁶ CD3-depleted and 0.35×10⁶ irradiated CD3+ cells/ml; iii) 400-900×10⁶ CD3-depleted and 400-900×10⁶ irradiated CD3+ cells per flask; or iv) 700×10⁶ CD3-depleted and 700×10⁶ irradiated CD3+ cells per flask.

The present disclosure provides methods for cryopreservation of a NK cell fraction comprising: (a) suspending an NK cell fraction of the present disclosure in a DMSO-free cryopreservation buffer; (b) adding DMSO to 10% v/v; (c) reducing temperature of the cells to −120° C.; and (d) storing the cryopreserved NK cell at a temperature of less than or equal to −120° C.

The present disclosure provides methods for preparing a thawed NK cell fraction comprising: (a) thawing a cryopreserved NK cell fraction of the present disclosure in a 37° C. water bath; (b) diluting said cryopreserved NK cell fraction thawed in step (a) with infusion solution thereby producing a thawed, cryopreserved NK cell fraction for transplantation.

The present disclosure provides methods of treating a hematological disease in a human subject in need thereof, the method comprising: (a) administering at least one anti-cancer monoclonal antibody to said subject; (b) administering at least one immunosuppressive agent to said subject; (c) administering an NK cell fraction of the present disclosure; and (d) administering IL-2 to said subject.

In some aspects, step (c) comprises administering a first dose of said NK cell fraction or thawed NK cell fraction and two days later administering a second dose of said NK cell fraction or thawed NK cell fraction.

In some aspects: (i) said first dose and said second dose each comprise at least about 1.25×10⁷ cells/kg, for a total dose of 2.5×10⁷ cells/kg; (ii) said first dose and said second dose each comprise at least about 2.5×10⁷ cells/kg, for a total dose of at least about 5×10⁷ cells/kg; (iii) said first dose and said second dose of said NK cell fraction each comprise 5×10⁷ cells/kg, for a total dose of 1×10⁸ cells/kg; or (iv) said first dose and said second dose of said NK cell fraction each comprise 1×10⁸ cells/kg, for a total dose of 2×10⁸ cells/kg.

In some aspects, said immunosuppressive agent is a chemotherapeutic immunosuppressive agent, irradiation or any combination thereof.

In some aspects, said hematological disease is a hematologic malignancy.

In some aspects, said hematological disease is multiple myeloma.

In some aspects, said hematological disease is non-Hodgkins lymphoma (NHL), In some aspects, the NHL is: i) CD20 positive B cell NHL; ii) follicular lymphoma (FL); iii) high grade B-cell lymphoma (HGBCL); iv) HGBCL, not otherwise specified (HGBCL, NOS); v) primary mediastinal large B-cell lymphoma (PMBCL); or vi) diffuse large B cell lymphoma (DLBCL).

In some aspects, said NHL is characterized by at least one of: a) relapsed/refractory disease that has failed conventional therapy; b) patient having received at least two prior lines of therapy, preferably wherein at least one of the at least two prior lines of therapy comprised the administration of chemotherapy and at least one of the at least two prior lines of therapy comprise the administration of an anti-CD20 monoclonal antibody; c) measurable disease as defined by the Lugano response criteria; and d) wherein said NLH is FL transformed to HGBCL, the subject has previously received at least one line of therapy after transformation to HGBCL.

In some aspects, said hematologic disease is NHL and said anticancer monoclonal antibody is rituximab, preferably wherein the rituximab is administered at least once at a dose of about 375 mg/m², preferably wherein rituximab is administered at least three times.

In some aspects, at least one anti-cancer monoclonal antibody is administered to the subject at least three times, preferably wherein the at least one anti-cancer monoclonal antibody is administered: at about 10 days before the first administration of the NK cell fraction or thawed NK cell fraction; at about 3 days before the first administration of the NK cell fraction or thawed NK cell fraction; and at 12-16 days following the first administration of the NK cell fraction or thawed NK cell fraction.

In some aspects, said at least one immunosuppressive agent comprises cyclophosphamide and/or fludarabine.

In some aspects, said at least one immunosuppressive agent comprises both cyclophosphamide (400 mg/m2) and fludarabine (30 mg/m²), wherein the cyclophosphamide is administered at a dose of 400 mg/m² and the fludarabine is administered at a dose of 30 mg/m², and wherein said cyclophosphamide and said fludarabine are administered: at about 5 days before the first administration of the NK cell fraction or thawed NK cell fraction; at about 4 days before the first administration of the NK cell fraction or thawed NK cell fraction; and at about 4 days before the first administration of the NK cell fraction or thawed NK cell fraction.

In some aspects, step (d) comprises administering 6×10⁶ units IL-2 one each one of following transfusion of said allogeneic thawed cryopreserved expanded NK cells: (i) on the day of the first administration of the NK cell fraction or thawed NK cell fraction; (ii) two days following the first administration of the NK cell fraction or thawed NK cell fraction; and (iii) four days following the first administration of the NK cell fraction or thawed NK cell fraction.

According to an aspect of some embodiments of the present invention there is provided a method of preparing an NK cell fraction for a subject in need thereof, the method comprising:
(a) obtaining an apheresis product comprising NK cells and CD3+ cells allogeneic to subject;
(b) separating the apheresis product into a CD3-depleted cell fraction and a CD3+ cell fraction;
(c) inactivating cells of the CD3+ cell fraction by irradiation;
(d) ex vivo culturing the CD3-depleted cell fraction with the inactivated, irradiated CD3+ cell fraction under conditions allowing for cell proliferation, wherein the conditions comprise providing nutrients, serum, IL-15, a CD3 agonist and nicotinamide in an amount between 1.0 mM to 10 mM;
(e) supplementing the combined CD3-depleted and CD3+ cell fractions with fresh nutrients, serum, IL-15 and nicotinamide 6-10 days following step (d) to produce an expanded CD3-depleted cell fraction;
(f) harvesting the combined CD3-depleted and CD3+ cell fractions 14-16 days following step (d); and
(g) washing and concentrating the combined CD3-depleted and CD3+ cell fraction of step (f),
thereby producing an expanded NK cell fraction for transplantation in the subject.

According to an aspect of some embodiments of the present invention there is provided an NK cell fraction prepared according to methods of the invention.

According to another aspect of some embodiments of the present invention there is provided a method for cryopreservation of NK cell fractions comprising:
(a) suspending NK cells of an NK cell fraction in a DMSO-free cryopreservation buffer;
(b) adding DMSO to 10% v/v,
(c) reducing temperature of the cells to −120° C., and
(d) storing the cryopreserved NK cell at <−120° C.

According to other aspects of some embodiments of the present invention there is provided a method for preparing a cryopreserved NK cell fraction for administration comprising
(a) thawing the cryopreserved NK cell fraction in a 37° C. water bath;
(b) diluting the cryopreserved NK cell fraction with infusion solution thereby producing a thawed, cryopreserved NK cell fraction for transplantation.

According to an aspect of some embodiments of the present invention there is provided a cryopreserved NK cell fraction prepared according to methods of the invention.

According to some embodiments of the present invention there is provided a thawed, cryopreserved NK cell fraction for administration (also referred to as transplantation) produced by the method of the invention, wherein the cryopreserved NK cell fraction is the cryopreserved NK cell fraction produced by the methods of the invention.

According to some embodiments of the present invention the cryopreserved NK cell fraction is a cryopreservation bag comprising 2.5×10⁸ total cells/ml in a volume of 20 ml. Thus, according to some embodiments of the present invention the cryopreserved NK cell fraction is a cryopreservation bag comprising 50×10⁸ cells in a volume of 20 ml.

According to some embodiments of the present invention, the expanded CD3-depleted cells of the thawed, cryopreserved NK cell fraction, have increased in-vivo retention in the spleen and bone marrow following infusion into irradiated mice compared to CD3-depleted cells expanded under identical conditions having 5 mM nicotinamide and without the CD3+ cell fraction.

According to some embodiments of the present invention, the thawed, cryopreserved NK cell fraction for transplantation is characterized by the following parameters:
(a) at least 70% CD56+/CD3− cells;
(b) at least 70% viability;
(c) fewer than 1×10⁶ CD3+/CD56− cells/Kg mass of patient, upon infusion;
(d) no more than 5 EU endotoxin/Kg mass of patient, upon infusion;
(e) no mycoplasma, and
(f) sterile.

According to some embodiments of the present invention, the thawed, cryopreserved NK cell fraction for transplantation is characterized by the following parameters:
(a) at least 70% CD56+/CD3− cells;
(b) at least 70% viability;
(c) less than 0.5% of cells are CD3+/CD56−;
(d) no more than 0.5 Endotoxin Units (EU)/mL;
(e) no mycoplasma, and
(f) sterile

According to some embodiments of the present invention the thawed cryopreserved NK cell fraction is provided in a fluorinated ethylene propylene (FEP) cryopreservation bag.

According to some embodiments of the present invention the thawed cryopreserved NK cell fraction, comprises 2.5×10⁸ cells/ml.

According to some embodiments of the present invention the thawed cryopreserved NK cell fraction is provided in a volume of 100 ml.

According to some embodiments of the present invention the apheresis product is a human apheresis product comprising human NK and CD3+ cell fractions.

According to some embodiments of the present invention, the serum is human serum.

According to some embodiments of the present invention the conditions for allowing for cell proliferation comprise providing 10% human serum.

According to some embodiments of the present invention the IL-15 comprises 20 ng/ml IL-15.

According to some embodiments of the present invention the CD3 agonist is OKT3, and the OKT3 comprises 1 μg/ml OKT3.

According to some embodiments of the present invention the nicotinamide comprises 7.0 mM nicotinamide.

According to some embodiments of the present invention the nutrients comprise minimal essential cell culture medium.

According to some embodiments of the present invention the NK cells of the expanded NK cell fraction comprise at least 40-97% CD56+/CD3− cells.

According to some embodiments of the present invention the washed and concentrated expanded NK cell fraction generated by step (e) of the method for of preparing an NK cell fraction for a subject in need thereof is characterized by the following parameters:
(a) at least 70% CD56+/CD3− cells;
(b) at least 70% viability;
(c) fewer than 1×10⁶ CD3+/CD56− cells/Kg mass of patient, upon infusion;
(d) no more than 5 EU endotoxin/Kg mass of patient, upon infusion;
(e) no mycoplasma, and
(f) sterile.

According to some embodiments of the present invention the washed and concentrated expanded NK cell fraction generated by step (e) of the method for of preparing an NK cell fraction for a subject in need thereof is characterized by the following parameters:
(a) at least 70% CD56+/CD3− cells;
(b) at least 70% viability;
(c) less than 0.5% of cells are CD3+/CD56−;
(d) no more than 0.5 Endotoxin Units (EU)/mL;
(e) no mycoplasma, and
(f) sterile

According to some embodiments of the present invention the CD3+ cells are fewer than 0.5× ×10⁶ CD3+/CD56− cells/Kg mass of patient, upon infusion. According

According to some embodiments of the present invention the CD3-depleted and the irradiated CD3+ cells are seeded at a 1:1 ratio.

According to some embodiments of the present invention, the culturing is affected in flasks at 0.30-0.40×10⁶ CD3-depleted and 0.30-0.40×10⁶ irradiated CD3+ cells/ml.

According to some embodiments of the present invention, culturing affected in flasks at 0.35×10⁶ CD3-depleted and 0.35×10⁶ irradiated CD3+ cells/ml.

According to some embodiments of the present invention, culturing is affected in flasks at 400-900×10⁶ CD3-depleted and 400-900×10⁶ irradiated CD3+ cells per flask.

According to some embodiments of the present invention culturing is affected in flasks at 700×10⁶ CD3-depleted and 700×10⁶ irradiated CD3+ cells per flask.

According to some embodiments of the present invention the expanded CD3-depleted cells have increased expression of CD62L compared to CD3-depleted cells expanded under identical conditions having 5 mM nicotinamide and without the CD3+ cell fraction.

According to some embodiments of the present invention the expanded CD3-depleted cells have increased antibody dependent cytotoxicity compared to CD3-depleted cells expanded under identical conditions having 5 mM nicotinamide and without the CD3+ cell fraction.

According to an aspect of some embodiments of the present invention there is provided a method of treating a hematological disease in a human subject in need thereof, the method comprising:
(a) administering an anti-cancer monoclonal antibody to the subject;
(b) administering at least one immunosuppressive agent to the subject;
(c) transplanting an allogeneic thawed, cryopreserved expanded NK cell fraction into the subject in need thereof, wherein the allogeneic thawed cryopreserved expanded NK cell fraction has been expanded by ex-vivo culturing with CD3+ cells, nutrients, serum, IL-15, a CD3 agonist and nicotinamide in an amount between 1.0 mM to 10 mM; and
(d) administering IL-2 to the subject,
thereby treating the hematologic disease in the subject.

According to some embodiments of the present invention the immunosuppressive agent is a chemotherapeutic immunosuppressive agent and/or irradiation.

According to some embodiments of the present invention the hematological disease is a hematologic malignancy. According to some embodiments of the present invention the hematological disease is non-Hodgkin's lymphoma (NHL).

According to some embodiments of the present invention the hematological disease is follicular lymphoma (FL) or a high-grade B-cell lymphoma (HGBCL). According to some embodiments of the invention, the hematological disease is diffuse large B cell lymphoma (DLBCL). According to some embodiments of the invention, the hematological disease is mantle cell lymphoma (MCL). According to some embodiments of the invention, the hematological disease is HGBCL, not otherwise specified (HGBCL, NOS). According to some embodiments of the invention, the hematological disease is primary mediastinal large B-cell lymphoma (PMBCL).

According to some embodiments of the present invention the NHL is CD20 positive B cell NHL.

According to some embodiments of the present invention the NHL is characterized by at least one of:
(a) relapsed/refractory disease that has failed conventional therapy;
(b) patient having received at least 2 prior lines of therapy (at least one of which contained chemotherapy and at least one of which contained an anti-CD20 monoclonal antibody);
c) measurable disease as defined by the Lugano response criteria;
d) wherein the NLH is FL transformed to HGBCL, must have received at least one line of therapy after transformation to HGBCL.

According to some embodiments of the present invention the hematologic malignancy is NHL, and the anticancer monoclonal antibody is rituximab (375 mg/m²).

According to some embodiments of the present invention administering an anti-cancer monoclonal antibody to the subject is performed three times.

According to some embodiments of the present invention the administering comprises administering a first dose of the allogeneic thawed cryopreserved expanded cell fraction followed two days later by a second dose of the allogeneic thawed cryopreserved expanded NK cell fraction.

According to some embodiments of the present invention administering the anti-cancer antibody is performed three times: at 10 days before the first dose, at 3 days before the first dose and at 12-16 days following the first dose of the allogeneic thawed cryopreserved expanded NK cell fraction.

According to some embodiments of the present invention the cell fraction comprises between 1×10⁷ and 5×10⁸ allogeneic thawed cryopreserved expanded NK cells per kg subject.

According to some embodiments of the present invention the cell fraction comprises between 2×10⁸ allogeneic thawed cryopreserved expanded NK cells per kg subject.

According to some embodiments of the present invention the combined the first and the second doses comprise 2×10⁷/kg to 2×10⁸/kg total allogeneic thawed cryopreserved expanded NK cells.

According to some embodiments of the present invention the
(a) the first dose and the second dose of the NK cell fraction each comprise 2.5×10⁷ allogeneic thawed cryopreserved expanded NK cells per kg subject, for a total dose of 5×10⁷ allogeneic thawed cryopreserved expanded NK cells per kg subject, or
(b) the first dose and the second dose of the NK cell fraction each comprise 5×10⁷ allogeneic thawed cryopreserved expanded NK cells per kg subject, for a total dose of 1×10⁸ allogeneic thawed cryopreserved expanded NK cells per kg subject, or
(c) the first dose and the second dose of the NK cell fraction each comprise 1×10⁸ allogeneic thawed cryopreserved expanded NK cells, for a total dose of 2×10⁸/kg allogeneic thawed cryopreserved expanded NK cells per kg subject.

According to some embodiments of the present invention the allogeneic thawed cryopreserved expanded NK cell fraction is administered to the subject no more than 4 hours after thawing.

According to some embodiments of the present invention the allogeneic thawed cryopreserved expanded NK cell fraction is administered to said subject by infusion, without a filter or pump, at a rate not greater than 10 cc/kg weight of the patient/hr.

According to some embodiments of the present invention the allogeneic thawed cryopreserved expanded NK cell fraction is administered to said subject by infusion, or pump, at a rate not greater than 10 cc/kg weight of the patient/hr.

According to some embodiments of the present invention the allogeneic thawed cryopreserved expanded NK cell fraction is administered to said subject by infusion, with a filter or pump, at a rate not greater than 10 cc/kg weight of the patient/hr. In some aspects the filter can have a pore size of about 170 to about 260 microns.

According to some embodiments of the present invention the at least one immunosuppressive agent comprises cyclophosphamide and/or fludarabine.

According to some embodiments of the present invention the
(i) said at least one immunosuppressive agent comprises both cyclophosphamide (400 mg/m²) and fludarabine (30 mg/m²); and
(ii) wherein said cyclophosphamide and said fludarabine are administered on each one of days 5, 4 and 3 days prior to transfusion of said allogeneic thawed cryopreserved expanded NK cells.

According to some embodiments of the present invention, administering (step (d)) comprises administering 6×10⁶ units IL-2 following transfusion of the allogeneic thawed cryopreserved expanded NK cells:
(i) on the day of transfusion of the allogeneic thawed cryopreserved expanded NK cells; and
(ii) two days following transfusion of the allogeneic thawed cryopreserved expanded NK cells; and
(iii) four days following transfusion of the allogeneic thawed cryopreserved expanded NK cells.

According to some embodiments of the present invention the method comprises transplanting a transplantable NK cell fraction prepared according to any of the methods.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting. Any aspect and/or embodiment described herein can be combined with any other aspect and/or embodiment described herein.

### BRIEF DESCRIPTION OF THE DRAWING(S)

The above and further features will be more clearly appreciated from the following detailed description when taken in conjunction with the accompanying drawings.

FIG. 1 is a histogram showing the retention of superior NK cell function in expanded cells cultured, cryopreserved and thawed according to the methods disclosed herein, as compared to cells cultured without nicotinamide or without irradiated feeder cells. Cells were cultured with 5 mM NAM without feeders, 5 mM NAM with feeders or 7 mM NAM with feeders and cryopreserved. ADCC assay was performed using labeled BL2 cells. Killing of BL2 cells was determined by FACS as a percentage of propidium-iodine (PI)-positive (dead) from the total labeled cells;

FIG. 2 is a histogram showing enhanced NK cell potency in expanded cells cultured, cryopreserved and thawed according to the methods disclosed herein, as compared to cells cultured without nicotinamide or without irradiated feeder cells. Cells were cultured for 2 weeks with 5 mM NAM without feeders, 5 mM NAM with feeders or 7 mM NAM with feeders and cryopreserved. The cells were thawed, and 2 hours post-thaw were sampled for staining against CD62L and analyzed by FACS.

FIG. 3 is a histogram showing the beneficial effect of culture, cryopreservation and thawing according to the methods disclosed herein on NK cell immune checkpoint receptors. Cells were cultured for 2 weeks without NAM and with 2.5 mM, 5 mM NAM or 7 mM NAM. Samples for staining against CD200R were analyzed by FACS.

FIG. 4 is a histogram showing the beneficial effects of culture, cryopreservation and thawing according to the methods disclosed herein on NK cell homing following infusion into irradiated NSG mice. CD3-depleted Cells were cultured with and without NAM. NSG mice were sub-lethally irradiated 24 hours prior to GDA-201 infusion and IL2 and IL15 IP injection. The cells were thawed, and 2 hours post-thaw infused to the mice. After 7 days mice were harvested and analyzed for human NK cells presence by staining for human CD45 APC and CD56 FITC. Dead cells were excluded using 7AAD dye.

FIGS. 5A and 5B are histograms illustrating the enhanced engraftment potential of the expanded cells cultured, cryopreserved and thawed according to the methods disclosed herein. Cells were cultured with 5 mM NAM without feeders, 5 mM NAM with feeders or 7 mM NAM with feeders and cryopreserved. NSG mice were sublethally irradiated 24 hours prior to NK infusion and IL2 and IL15 IP injection. The cells were thawed and 2 hours post-thaw infused to the mice. After 3 days mice were harvested and analyzed for human NK cells presence by staining for human CD45 APC and CD56 FITC. Dead cells were excluded using 7AAD dye. 5A: Absolute number of NK events in spleen. 5B: Fraction of NK in bone marrow.

FIGS. 6A-6C show reduction of solid tumor volume in the mouse lung carcinoma A549 model by administration of the expanded cells cultured, cryopreserved and thawed according to the methods disclosed herein. Cells were cultured with 7 mM NAM with CD3+ feeders, and then administered IV to sub-lethally irradiated mice receiving 5×10⁶ A549 cells with or without anti-Her2 antibody Herceptin) (FIGS. 6A and 6B). Administration of the NK cells is clearly effective in reducing the tumor load (6C, C-NK), and synergistic when combined with the anti-Her2 antibody (6C, D-NK+Her2).

FIGS. 7A-7C show enhanced reduction of solid tumor volume in the mouse lung carcinoma A549 model by repeated administration of the expanded cells cultured, cryopreserved and thawed according to the methods disclosed herein. Cells were cultured with 7 mM NAM with CD3+ feeders, and then administered IV to sub-lethally irradiated mice receiving 5×10⁶ A549 cells with or without anti-Her2 antibody Herceptin) (FIGS. 7A and 7B). Multiple administration of the NK cells is clearly more effective in reducing the tumor load (7C, C-NK), and strongly synergistic when combined with the anti-Her2 antibody (6C, D-NK+Her2).

FIG. 8 is a series of graphs showing the expression of various cell surface markers in cells produced using the methods of the present disclosure, in the absence of nicotinamide (NAM0) or the presence of 7 mM nicotinamide (NAM7) as measured by FACs.

FIG. 9 is a series of representative FACs plots showing the expression of various cell surface markers in cells produced using the methods of the present disclosure, in the absence of nicotinamide (NAM0) or the presence of 7 mM nicotinamide (NAM7) as measured by FACs.

### DETAILED DESCRIPTION

The present invention is of methods of expanding natural killer (NK) cells from a CD3-depleted cell fraction for administration (also referred to herein as transplantation) by infusion into a subject, which can be efficiently cryopreserved and thawed, while at the same time, maintaining or enhancing function of the cells ex-vivo and/or in-vivo. In one embodiment, ex-vivo culture of a CD3-depleted fraction of an apheresis product comprising NK cells with a nicotinamide and/or other nicotinamide moiety, NK cell growth factors and inactivated cells from the corresponding CD3+ fraction facilitates the production of NK cell populations for use as a therapeutic ex-vivo expanded NK cell preparation, which includes an expanded population of functional NK cells having parameters suitable for infusion into a subject (e.g. robust expansion of NK cells alongside a reduced CD3+ T cell fraction), retaining superior viability and function with cryopreservation. Specifically in this respect, the present invention can be used to provide NK cell fractions and protocols for their cryopreservation and use, which can be employed for applications in cell transplants and infusions for treatment of cancer and other disease. Non-limiting applications may include allogeneic adoptive immunotherapy and combination immunotherapy along with sensitizing agents and other anti-cancer modalities.

The principles and operation of the present invention may be better understood with reference to the accompanying descriptions.

Before explaining at least one embodiment of the invention in detail, it is to be understood that the invention is not necessarily limited in its application to the details set forth in the following description. The invention is capable of other embodiments or of being practiced or carried out in various ways.

Natural killer (hereinafter also abbreviated as “NK”) cells are lymphoid cells that participate in immune reactions, exhibiting spontaneous non-MHC-restricted cytotoxic activity against tumor cells. Thus, developing clinical-grade protocols for effectively ex-vivo expanding the number of viable NK cells and effectively enhancing their function and suitability for cryopreservation, as well as likelihood of homing to lymph nodes and their homeostatic proliferation in-vivo following infusion, could improve the success of adoptive immunotherapy with NK cells for the treatment of cancerous conditions, such as solid tumors, hematopoietic malignancies and the like.

The present invention provides clinically appropriate conditions for preparing and characterizing expanded NK cell fractions suitable for transplantation by infusion, in the clinical setting, based on culturing a CD3-depleted cell fraction comprising NK cells with nicotinamide, together with inactivated cells of the corresponding CD3+ fraction, above a certain concentration of nicotinamide, and further cryopreserving the expanded cell fraction, as is further detailed herein. As such, in embodiments thereof, the present invention provides clinically appropriate culture conditions for production of an expanded NK cell fraction of functionally mature NK cells, with minimal non-NK cell (e.g. CD3+/CD56−) proliferation, therapeutic NK fractions and criteria for their selection, cryopreservation and thawing, as well as clinical protocols for their use in treatment of cancerous disease, in particular, hematological malignancies.

### NK Cell Fractions of the Present Disclosure

The present disclosure provides compositions comprising an NK cell fraction comprising a population of nucleated cells.

In some aspects, the population of nucleated cells can comprise at least about 1.0×10⁶, or at least about 5.0×10⁶, or at least about 1.0×10⁷, or at least about 5.0×10⁷, or at least about 1.0×10⁸, or at least about 5.0×10⁸, or at least about 1.0×10⁹, or at least about 5.0×10⁹, or at least about 1.0×10¹⁰, or at least about 5.0×10¹⁰, or at least about 1.0×10¹¹, or at least about 5.0×10¹¹, or at least about 1.0×10¹², or at least about 5.0×10¹² nucleated cells. In some aspects, the population of nucleated cells can comprise at least about at least about 1.0×10⁶ cells. In some aspects, the population of nucleated cells can comprise at least about at least about 17.5×10⁸ cells. In some aspects, the population of nucleated cells can comprise at least about at least about 35×10⁸. In some aspects, the population of nucleated cells can comprise at least about at least about 2.5×10⁹ cells. In some aspects, the population of nucleated cells can comprise at least about at least about 5×10⁹ cells.

In some aspects, at least about 60%, or at least about 65%, or at least about 70%, or at least about 75%, or at least about 80%, or at least about 85%, or at least about 90%, or at least about 95%, or at least about 99% of the cells in the population of nucleated cells are viable. In some aspects, at least about 70% of the cells in the population of nucleated cells are viable.

In some aspects, at least about at least about 60%, or at least about 65%, or at least about 70%, or at least about 75%, or at least about 80%, or at least about 85%, or at least about 90%, or at least about 95%, or at least about 99% of the cells in the population of nucleated cells are CD56+. In some aspects, at least about 70% of cells in the population of nucleated cells are CD56+.

In some aspects, about 80% to about 99%, or about 85% to about 95%, or about 90 to about 95% of the cells in the population of nucleated cells are CD56+. In some aspects, about 90 to about 95% of the cells in the population of nucleated cells are CD56+.

In some aspects, no more than about 0.1%, or no more than about 0.2%, or no more than about 0.3%, or no more than about 0.4%, or no more than about 0.5%, or no more than about 0.6%, or no more than about 0.7%, or no more than about 0.8%, or no more than about 0.9%, or no more than about 1.0% of cells in the population of nucleated cells are CD3+. In some aspects, no more than 0.5% of cells in the population of nucleated cells are CD3+.

In some aspects, about 0.01% to about 0.1%, or about 0.01% to about 0.2%, or about 0.01% to about 0.3%, or about 0.01% to about 0.4%, or about 0.01% to about 0.5%, or about 0.01% to about 0.6%, or about 0.01% to about 0.7%, or about 0.01% to about 0.8%, or about 0.01% to about 0.9%, or about 0.01% to about 1.0% of cells in the population of nucleated cells CD3+. In some aspects, about 0.01% to about 0.5% of the cells in the population of nucleated cells are CD3+.

In some aspects, about 0.1% to about 0.5%, or about 0.2% to about 0.3% of cells in the population of nucleated cells are CD3+. In some aspects, about 0.2% to about 0.3% of cells in the population of nucleated cells are CD3+.

In some aspects, at least about 60%, or at least about 65%, or at least about 70%, or at least about 75%, or at least about 80%, or at least about 85%, or at least about 90%, or at least about 95%, or at least about 99% of the cells in the population of nucleated cells are CD56+/CD3−. In some aspects, at least about 70% of cells in the population of nucleated cells are CD56+/CD3−. In some aspects, at least about 99% of the cells in the population of nucleated cells are CD56+/CD3−.

In some aspects, about 80% to about 99%, or about 85% to about 95%, or about 90 to about 95% of the cells in the population of nucleated cells is CD56+/CD3−. In some aspects, about 90 to about 95% of the cells in the population of nucleated cells is CD56+/CD3−.

In some aspects, no more than about 0.1%, or no more than about 0.2%, or no more than about 0.3%, or no more than about 0.4%, or no more than about 0.5%, or no more than about 0.6%, or no more than about 0.7%, or no more than about 0.8%, or no more than about 0.9%, or no more than about 1.0% of cells in the population of nucleated cells are CD56−/CD3+. In some aspects, no more than 0.5% of cells in the population of nucleated cells are CD56−/CD3+.

In some aspects, about 0.01% to about 0.1%, or about 0.01% to about 0.2%, or about 0.01% to about 0.3%, or about 0.01% to about 0.4%, or about 0.01% to about 0.5%, or about 0.01% to about 0.6%, or about 0.01% to about 0.7%, or about 0.01% to about 0.8%, or about 0.01% to about 0.9%, or about 0.01% to about 1.0% of cells in the population of nucleated cells are CD56−/CD3+. In some aspects, about 0.01% to 0.5% of cells in the population of nucleated cells are CD56−/CD3+.

In some aspects, about 0.1% to about 0.5%, or about 0.2% to about 0.3% of cells in the population of nucleated cells are CD56−/CD3+. In some aspects, about 0.2% to about 0.3% of cells in the population of nucleated cells are CD56−/CD3+.

In some aspects, at least about 5%, or at least about 10%, or at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75% of cells in the population of nucleated cells are CD62L+. In some aspects, at least about 10% of the cells in the population of nucleated cells are CD62L+. In some aspects, at least about 15% of the cells in the population of nucleated cells are CD62L+. In some aspects, at least about 13% of cells in the population of nucleated cells are CD62L+.

In some aspects, about 8% to about 80%, or about 13% to about 75%, or about 18% to about 70% of cells in the population of nucleated cells are CD62L+. In some aspects, about 13% to about 88% of cells in the population of nucleated cells are CD62L+.

In some aspects, at least about 5%, or at least about 10%, or at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75% of the cells in the population of nucleated cells are CD16+. In some aspects, at least about 10% of the cells in the population of nucleated cells are CD16+. In some aspects, at least about 20% of the cells in the population of nucleated cells are CD16+. In some aspects, at least 30% of the cells in the population of nucleated cells are CD16+.

In some aspects, about 10% to about 70%, or about 15% to about 65%, or about 20% to about 60% of cells in the population of nucleated cells are CD16+. In some aspects, about 20% to about 60% of cells in the population of nucleated cells are CD16+. In some aspects, about 30% to about 88% of cells in the population of nucleated cells are CD16+.

In some aspects, no more than about 5%, or no more than about 10%, or no more than about 15%, or no more than about 20%, or no more than about 25% of cells in the population of nucleated cells are CD19+. In some aspects, no more than about 10% of cells in the population of nucleated cells are CD19+. In some aspects, no more than about 0.7% of the cells in the population of nucleated cells are CD19+.

In some aspects, about 0.01% to about 5%, or about 0.01% about 10%, or about 0.01% to about 15%, or about 0.01% to about 20%, or about 0.01% to about 25% of cells in the population of nucleated cells are CD19+. In some aspects, about 0.01% to about 10% of cells in the population of nucleated cells are CD19+. In some aspects, about 0.01% to about 0.7% of the cells in the population of nucleated cells are CD19+.

In some aspects, about 0.1% to about 5%, or about 0.1% about 10%, or about 0.1% to about 15%, or about 0.1% to about 20%, or about 0.1% to about 25% of cells in the population of nucleated cells are CD19+. In some aspects, about 0.1% to about 10% of cells in the population of nucleated cells are CD19+. In some aspects, about 0.1% to about 0.7% of the cells in the population of nucleated cells are CD19+.

In some aspects, no more than about 5%, or no more than about 10%, or no more than about 15%, or no more than about 20%, or no more than about 25% of cells in the population of nucleated cells are CD14+. In some aspects, no more than about 10% of cells in the population of nucleated cells are CD14+. In some aspects, no more than about 0.05% of the cells in the population of nucleated cells are CD14+.

In some aspects, about 0.01% to about 5%, or about 0.01% to about 10%, or about 0.01% to about 15%, or about 0.01% to about 20%, or about 0.01% to about 25% of cells in the population of nucleated cells are CD14+. In some aspects, about 0.01% to about 10% of cells in the population of nucleated cells are CD14+. In some aspects, about 0.01% to about 0.05% of the cells in the population of nucleated cells are CD14+.

In some aspects, about 0.1% to about 5%, or about 0.1% to about 10%, or about 0.1% to about 15%, or about 0.1% to about 20%, or about 0.1% to about 25% of cells in the population of nucleated cells are CD14+. In some aspects, about 0.1% to about 10% of cells in the population of nucleated cells are CD14+. In some aspects, about 0.1% to about 0.05% of the cells in the population of nucleated cells are CD14+.

In some aspects, at least about 44.99% of the cells in the population of nucleated cells are CD49a+. In some aspects, at least about 45% of the cells in the population of nucleated cells are CD49a+. In some aspects, at least about 96% of the cells in the population of nucleated cells are CD49a+. In some aspects, at least about 35%, or at least about 40%, or at least about 45%, or at least about 50%, or at least about 50%, or at least about 55%, or at least about 60%, or at least about 65%, or at least about 70%, or at least about 75%, or at least about 80%, or at least about 85%, or at least about 90%, or at least about 95% CD49a+. In some aspects, about 44% to about 96% of the cells in the population of nucleated cells are CD49a+.

In some aspects, no more than 26.39% of the cells in the population of nucleated cells are NKp80+. In some aspects, no more than about 27% of the cells in the population of nucleated cells are NKp80+. In some aspects, no more than about 76.68% of cells in the population of nucleated cells are NKp80+. In some aspects, no more than about 76% of cells in the population of nucleated cells are NKp80+. In some aspects, no more than about 25%, or no more than about 30%, or no more than about 35%, or no more than about 40%, or no more than about 45%, or no more than about 50%, or no more than about 55%, or no more than about 60%, or no more than about 65%, or no more than about 70%, or no more than about 75%, or no more than about 77%, or no more than about 80% of cells in the population of nucleated cells are NKp80+. In some aspects, about 26% to about 77% of the cells in the population of nucleated cells are NKp80+.

In some aspects, about 0.01% to about 26.39% of the cells in the population of nucleated cells are NKp80+. In some aspects, about 0.01% to about 27% of the cells in the population of nucleated cells are NKp80+. In some aspects, about 0.01% to about 76.68% of cells in the population of nucleated cells are NKp80+. In some aspects, about 0.01% to about 76% of cells in the population of nucleated cells are NKp80+. In some aspects, about 0.01% to about 25%, or about 0.01% to about 30%, or about 0.01% to about 35%, or about 0.01% to about 40%, or about 0.01% to about 45%, or about 0.01% to about 50%, or about 0.01% to about 55%, or about 0.01% to about 60%, or about 0.01% to about 65%, or about 0.01% to about 70%, or about 0.01% to about 75%, or about 0.01% to about 77%, or about 0.01% to about 80% of cells in the population of nucleated cells are NKp80+.

In some aspects, about 0.1% to about 26.39% of the cells in the population of nucleated cells are NKp80+. In some aspects, about 0.1% to about 27% of the cells in the population of nucleated cells are NKp80+. In some aspects, about 0.1% to about 76.68% of cells in the population of nucleated cells are NKp80+. In some aspects, about 0.1% to about 76% of cells in the population of nucleated cells are NKp80+. In some aspects, about 0.1% to about 25%, or about 0.01% to about 30%, or about 0.01% to about 35%, or about 0.1% to about 40%, or about 0.1% to about 45%, or about 0.1% to about 50%, or about 0.1% to about 55%, or about 0.1% to about 60%, or about 0.1% to about 65%, or about 0.1% to about 70%, or about 0.1% to about 75%, or about 0.1% to about 77%, or about 0.1% to about 80% of cells in the population of nucleated cells are NKp80+.

In some aspects, no more than about 0.57% of the cells in the population of nucleated cells are LAG3+. In some aspects, no more than about 1% of the cells in the population of nucleated cells are LAG3+. In some aspects, no more than about 2% of the cells in the population of nucleated cells are LAG3+. In some aspects, no more than about 26.89% of the cells in the population of nucleated cells are LAG3+. In some aspects, no more than about 27% of the cells in the population of nucleated cells are LAG3+. In some aspects, no more than about 2.5%, or no more than about 5%, or no more than about 10%, or no more than about 15%, or no more than about 20%, or no more than about 30%, or no more than about 35%, or no more than about 40% of cells in the population of nucleated cells are LAG3+. In some aspects, no more than about 10% of cells in the population of nucleated cells are LAG3+. In some aspects, about 0.5% to about 27% of cells in the population of nucleated cells are LAG3+.

In some aspects, about 0.01% to about 0.57% of the cells in the population of nucleated cells are LAG3+. In some aspects, about 0.01% to about 1% of the cells in the population of nucleated cells are LAG3+. In some aspects, about 0.01% to about 2% of the cells in the population of nucleated cells are LAG3+. In some aspects, about 0.01% to about 26.89% of the cells in the population of nucleated cells are LAG3+. In some aspects, about 0.01% to about 27% of the cells in the population of nucleated cells are LAG3+. In some aspects, about 0.01% to about 2.5%, or about 0.01% to about 5%, or about 0.01% to about 10%, or about 0.01% to about 15%, or about 0.01% to about 20%, or about 0.01% to about 30%, or about 0.01% to about 35%, or about 0.01% to about 40% of cells in the population of nucleated cells are LAG3+. In some aspects, about 0.01% to about 10% of cells in the population of nucleated cells are LAG3+.

In some aspects, about 0.1% to about 0.57% of the cells in the population of nucleated cells are LAG3+. In some aspects, about 0.1% to about 1% of the cells in the population of nucleated cells are LAG3+. In some aspects, about 0.1% to about 2% of the cells in the population of nucleated cells are LAG3+. In some aspects, about 0.1% to about 26.89% of the cells in the population of nucleated cells are LAG3+. In some aspects, about 0.1% to about 27% of the cells in the population of nucleated cells are LAG3+. In some aspects, about 0.1% to about 2.5%, or about 0.1% to about 5%, or about 0.1% to about 10%, or about 0.1% to about 15%, or about 0.1% to about 20%, or about 0.1% to about 30%, or about 0.1% to about 35%, or about 0.1% to about 40% of cells in the population of nucleated cells are LAG3+. In some aspects, about 0.1% to about 10% of cells in the population of nucleated cells are LAG3+.

In some aspects, no more than about 1.03% of cells in the population of nucleated cells is CD200R+. In some aspects, no more than about 2% of the cells in the population of nucleated cells is CD200R+. In some aspects, no more than about 3.68% of cells in the population of nucleated cells is CD200R+. In some aspects, no more than about 31.8% of cells in the population of nucleated cells is CD200R+. In some aspects, no more than about 32% of the cells in the population of nucleated cells is CD200R+. In some aspects, no more than about 2.5%, or no more than about 5%, or no more than about 10%, or no more than about 15%, or no more than about 20%, or no more than about 30%, or no more than about 35%, or no more than about 40% of cells in the population of nucleated cells are CD200R+. In some aspects, no more than about 10% of cells in the population of nucleated cells are CD200R+. In some aspects, about 1% to about 32% of cells in the population of cells are CD200R+.

In some aspects, about 0.01% to about 1.03% of cells in the population of nucleated cells is CD200R+. In some aspects, about 0.01% to about 2% of the cells in the population of nucleated cells is CD200R+. In some aspects, about 0.01% to about 3.68% of cells in the population of nucleated cells is CD200R+. In some aspects, about 0.01% to about 31.8% of cells in the population of nucleated cells is CD200R+. In some aspects, about 0.01% to about 32% of the cells in the population of nucleated cells is CD200R+. In some aspects, about 0.01% to about 2.5%, or about 0.01% to about 5%, or about 0.01% to about 10%, or about 0.01% to about 15%, or about 0.01% to about 20%, or about 0.01% to about 30%, or about 0.01% to about 35%, or about 0.01% to about 40% of cells in the population of nucleated cells are CD200R+. In some aspects, about 0.01% to about 10% of cells in the population of nucleated cells are CD200R+.

In some aspects, about 0.1% to about 1.03% of cells in the population of nucleated cells is CD200R+. In some aspects, about 0.1% to about 2% of the cells in the population of nucleated cells is CD200R+. In some aspects, about 0.1% to about 3.68% of cells in the population of nucleated cells is CD200R+. In some aspects, about 0.1% to about 31.8% of cells in the population of nucleated cells is CD200R+. In some aspects, about 0.1% to about 32% of the cells in the population of nucleated cells is CD200R+. In some aspects, about 0.1% to about 2.5%, or about 0.1% to about 5%, or about 0.1% to about 10%, or about 0.1% to about 15%, or about 0.1% to about 20%, or about 0.1% to about 30%, or about 0.1% to about 35%, or about 0.1% to about 40% of cells in the population of nucleated cells are CD200R+. In some aspects, about 0.1% to about 10% of cells in the population of nucleated cells are CD200R+.

In some aspects, no more than about 2.05% of cells in the population of nucleated cells are CD57+. In some aspects, no more than about 2.5% of cells in the population of cells is CD57+. In some aspects, no more than about 3.6% of cells in the population of cells is CD57+. In some aspects, no more than about 4% of cells in the population of cells is CD57+. In some aspects, no more than about 24.59% of cells in the population of cells is CD57+. In some aspects, no more than about 25% of cells in the population of cells is CD57+. In some aspects, no more than about 2.5%, or no more than about 5%, or no more than about 10%, or no more than about 15%, or no more than about 20%, or no more than about 30%, or no more than about 35%, or no more than about 40% of cells in the population of nucleated cells are CD57+. In some aspects, no more than about 10% of cells in the population of nucleated cells are CD57+. In some aspects, about 2% to about 25% of cells in the population of nucleated cells are CD57+.

In some aspects, about 0.01% to about 2.05% of cells in the population of nucleated cells are CD57+. In some aspects, about 0.01% to about 2.5% of cells in the population of cells is CD57+. In some aspects, about 0.01% to about 3.6% of cells in the population of cells is CD57+. In some aspects, about 0.01% to about 4% of cells in the population of cells is CD57+. In some aspects, about 0.01% to about 24.59% of cells in the population of cells is CD57+. In some aspects, about 0.01% to about 25% of cells in the population of cells is CD57+. In some aspects, about 0.01% to about 2.5%, or about 0.01% to about 5%, or about 0.01% to about 10%, or about 0.01% to about 15%, or about 0.01% to about 20%, or about 0.01% to about 30%, or about 0.01% to about 35%, or about 0.01% to about 40% of cells in the population of nucleated cells are CD57+. In some aspects, about 0.01% to about 10% of cells in the population of nucleated cells are CD57+.

In some aspects, about 0.1% to about 2.05% of cells in the population of nucleated cells are CD57+. In some aspects, about 0.1% to about 2.5% of cells in the population of cells is CD57+. In some aspects, about 0.1% to about 3.6% of cells in the population of cells is CD57+. In some aspects, about 0.1% to about 4% of cells in the population of cells is CD57+. In some aspects, about 0.1% to about 24.59% of cells in the population of cells is CD57+. In some aspects, about 0.1% to about 25% of cells in the population of cells is CD57+. In some aspects, about 0.1% to about 2.5%, or about 0.1% to about 5%, or about 0.1% to about 10%, or about 0.1% to about 15%, or about 0.1% to about 20%, or about 0.1% to about 30%, or about 0.1% to about 35%, or about 0.1% to about 40% of cells in the population of nucleated cells are CD57+. In some aspects, about 0.1% to about 10% of cells in the population of nucleated cells are CD57+.

In some aspects, no more than about 1%, or no more than about 2%, or no more than about 3%, or no more than about 4%, or no more than about 5%, or no more than about 6%, or no more than about 7%, or no more than about 8%, or no more than about 9%, or no more than about 10%, or no more than about 11%, or no more than about 12%, or no more than about 13%, or no more than about 14%, or no more than about 15%, or no more than about 16%, or no more than about 17%, or no more than about 18%, or no more than about 19%, or no more than about 20% of cells in the population of nucleated cells are CD56+/LAG3+. In some aspects, no more than about 3% of the cells in the population of nucleated cells are CD56+/LAG3+.

In some aspects, no more than about 1%, or no more than about 2%, or no more than about 3%, or no more than about 4%, or no more than about 5%, or no more than about 6%, or no more than about 7%, or no more than about 8%, or no more than about 9%, or no more than about 10%, or no more than about 11%, or no more than about 12%, or no more than about 13%, or no more than about 14%, or no more than about 15%, or no more than about 16%, or no more than about 17%, or no more than about 18%, or no more than about 19%, or no more than about 20% of cells in the population of nucleated cells are CD56+/CD200R+. In some aspects, no more than about 11% of cells in the population of nucleated cells are CD56+/CD200R+.

In some aspects, no more than about 1%, or no more than about 2%, or no more than about 3%, or no more than about 4%, or no more than about 5%, or no more than about 6%, or no more than about 7%, or no more than about 8%, or no more than about 9%, or no more than about 10%, or no more than about 11%, or no more than about 12%, or no more than about 13%, or no more than about 14%, or no more than about 15%, or no more than about 16%, or no more than about 17%, or no more than about 18%, or no more than about 19%, or no more than about 20% of cells in the population of nucleated cells are CD56+/CD57+. In some aspects, no more than about 4% of the cells in the population of nucleated cells are CD56+/CD57+.

In some aspects, about 0.01% to about 1%, or about 0.01% to about 2%, or about 0.01% to about 3%, or about 0.01% to about 4%, or about 0.01% to about 5%, or about 0.01% to about 6%, or about 0.01% to about 7%, or about 0.01% to about 8%, or about 0.01% to about 9%, or about 0.01% to about 10%, or about 0.01% to about 11%, or about 0.01% to about 12%, or about 0.01% to about 13%, or about 0.01% to about 14%, or about 0.01% to about 15%, or about 0.01% to about 16%, or about 0.01% to about 17%, or about 0.01% to about 18%, or about 0.01% to about 19%, or about 0.01% to about 20% of cells in the population of nucleated cells are CD56+/LAG3+. In some aspects, about 0.01% to about 3% of the cells in the population of nucleated cells are CD56+/LAG3+.

In some aspects, about 0.01% to about 1%, or about 0.01% to about 2%, or about 0.01% to about 3%, or about 0.01% to about 4%, or about 0.01% to about 5%, or about 0.01% to about 6%, or about 0.01% to about 7%, or about 0.01% to about 8%, or about 0.01% to about 9%, or about 0.01% to about 10%, or about 0.01% to about 11%, or about 0.01% to about 12%, or about 0.01% to about 13%, or about 0.01% to about 14%, or about 0.01% to about 15%, or about 0.01% to about 16%, or about 0.01% to about 17%, or about 0.01% to about 18%, or about 0.01% to about 19%, or about 0.01% to about 20% of cells in the population of nucleated cells are CD56+/CD200R+. In some aspects, about 0.01% to about 11% of cells in the population of nucleated cells are CD56+/CD200R+.

In some aspects, about 0.01% to about 1%, or about 0.01% to about 2%, or about 0.01% to about 3%, or about 0.01% to about 4%, or about 0.01% to about 5%, or about 0.01% to about 6%, or about 0.01% to about 7%, or about 0.01% to about 8%, or about 0.01% to about 9%, or about 0.01% to about 10%, or about 0.01% to about 11%, or about 0.01% to about 12%, or about 0.01% to about 13%, or about 0.01% to about 14%, or about 0.01% to about 15%, or about 0.01% to about 16%, or about 0.01% to about 17%, or about 0.01% to about 18%, or about 0.01% to about 19%, or about 0.01% to about 20% of cells in the population of nucleated cells are CD56+/CD57+. In some aspects, about 0.01% to about 4% of the cells in the population of nucleated cells are CD56+/CD57+.

In some aspects, at least about 30%, or at least about 35%, or at least about 40%, or at least about 45%, or at least about 50%, or at least about 55%, or at least about 60%, or at least about 65%, or at least about 70% of cells in the population of nucleated cells are CD56+/CD16+. In some aspects, at least about 43% of the cells in the population of nucleated cells are CD56+/CD16+. In some aspects, at least about 57% of the cells in the population of nucleated cells are CD56+/CD16+.

In some aspects, at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85% of cells in the population of nucleated cells are CD56+/CD62L+. In some aspects, at least about 78% of cells in the population of nucleated cells are CD56+/CD62L+.

Any of the aforementioned phenotypic parameters can be combined with any of the other aforementioned phenotypic parameters.

Accordingly, in a non-limiting example, the present disclosure provides NK cell fractions comprising a population of nucleated cells, wherein the population comprises at least 1.0×10⁶ nucleated cells, wherein at least about 70% of the cells in the population are viable, wherein:
at least about 70% of the cells in the population are CD56+;
no more than about 0.5% of the cells in the population are CD3+
no more than about 10% of the cells in the population are CD19+;
no more than about 10% of the cells in the population are CD14+;
at least about 44% of the cells in the population are CD49a+;
no more than about 10% of the cells in the population are LAG3+;
no more than about 20% of the cells in the population are CD200R+;
no more than about 10% of the cells in the population are CD57+;
at least about 10% of the cells in the population are CD16+; and
at least about 10% of the cells in the population are CD62L+.

Accordingly, in a non-limiting example, the present disclosure provides NK cell fractions comprising a population of nucleated cells, wherein the population comprises at least 1.0×10⁶ nucleated cells, wherein at least about 70% of the cells in the population are viable, wherein:
at least about 70% of the cells in the population are CD56+;
no more than about 0.5% of the cells in the population are CD3+
no more than about 1% of the cells in the population are CD19+;
no more than about 0.05% of the cells in the population are CD14+;
at least about 44% of the cells in the population are CD49a+;
no more than about 27% of the cells in the population are LAG3+;
no more than about 32% of the cells in the population are CD200R+;
no more than about 25% of the cells in the population are CD57+;
at least about 30% of the cells in the population are CD16+; and
at least about 13% of the cells in the population are CD62L+.

Accordingly, in a non-limiting example, the present disclosure provides NK cell fractions comprising a population of nucleated cells, wherein the population comprises at least 1.0×10⁶ nucleated cells, wherein at least about 70% of the cells in the population are viable, wherein:
at least about 70% of the cells in the population are CD56+;
about 0.01% to about 0.5% of the cells in the population are CD3+
about 0.01% to about 10% of the cells in the population are CD19+;
about 0.01% to about 10% of the cells in the population are CD14+;
at least about 44% of the cells in the population are CD49a+;
about 0.01% to about 10% of the cells in the population are LAG3+;
about 0.01% to about 20% of the cells in the population are CD200R+;
about 0.01% to about 10% of the cells in the population are CD57+;
at least about 10% of the cells in the population are CD16+; and
at least about 10% of the cells in the population are CD62L+.

Accordingly, in a non-limiting example, the present disclosure provides NK cell fractions comprising a population of nucleated cells, wherein the population comprises at least 1.0×10⁶ nucleated cells, wherein at least about 70% of the cells in the population are viable, wherein:
at least about 70% of the cells in the population are CD56+;
about 0.01% to about 0.5% of the cells in the population are CD3+
about 0.01% to about 1% of the cells in the population are CD19+;
about 0.01% to about 0.05% of the cells in the population are CD14+;
at least about 44% of the cells in the population are CD49a+;
about 0.01% to about 27% of the cells in the population are LAG3+;
about 0.01% to about 32% of the cells in the population are CD200R+;
about 0.01% to about 25% of the cells in the population are CD57+;
at least about 30% of the cells in the population are CD16+; and
at least about 13% of the cells in the population are CD62L+.

Accordingly, in a non-limiting example, the present disclosure provides NK cell fractions comprising a population of nucleated cells, wherein the population comprises at least 1.0×10⁶ nucleated cells, wherein at least about 70% of the cells in the population are viable, wherein:
at least about 70% of the cells in the population are CD56+;
about 0.1% to about 0.5% of the cells in the population are CD3+
about 0.1% to about 10% of the cells in the population are CD19+;
about 0.1% to about 10% of the cells in the population are CD14+;
at least about 44% of the cells in the population are CD49a+;
about 0.1% to about 10% of the cells in the population are LAG3+;
about 0.1% to about 20% of the cells in the population are CD200R+;
about 0.1% to about 10% of the cells in the population are CD57+;
at least about 10% of the cells in the population are CD16+; and
at least about 10% of the cells in the population are CD62L+.

Accordingly, in a non-limiting example, the present disclosure provides NK cell fractions comprising a population of nucleated cells, wherein the population comprises at least 1.0×10⁶ nucleated cells, wherein at least about 70% of the cells in the population are viable, wherein:
at least about 70% of the cells in the population are CD56+;
about 0.1% to about 0.5% of the cells in the population are CD3+
about 0.1% to about 1% of the cells in the population are CD19+;
about 0.1% to about 0.05% of the cells in the population are CD14+;
at least about 44% of the cells in the population are CD49a+;
about 0.1% to about 27% of the cells in the population are LAG3+;
about 0.1% to about 32% of the cells in the population are CD200R+;
about 0.1% to about 25% of the cells in the population are CD57+;
at least about 30% of the cells in the population are CD16+; and
at least about 13% of the cells in the population are CD62L+.

Accordingly, in a non-limiting example, the present disclosure provides NK cell fractions comprising a population of nucleated cells, wherein the population comprises at least 1.0×10⁶ nucleated cells, wherein at least about 70% of the cells in the population are viable, wherein:
at least about 70% of the cells in the population are CD56+;
no more than about 0.5% of the cells in the population are CD3+
about 0% to about 1% of the cells in the population are CD19+;
about 0% to about 0.05% of the cells in the population are CD14+;
about 44% to about 96% of the cells in the population are CD49a+;
about 0.5% to about 27% of the cells in the population are LAG3+;
about 1% to about 32% of the cells in the population are CD200R+;
about 2% to about 25% of the cells in the population are CD57+;
about 30% to about 88% of the cells in the population are CD16+; and
about 13% to about 88% of the cells in the population are CD62L+.

Other non-limiting examples of combinations of phenotypic parameters of the NK cell fractions of the present disclosure are presented in Table A-Table D.

TABLE A

| Phenotypic Parameter | % of nucleated cells |
|---|---|
| CD3−/CD56+ | ≥70% |
| CD3+/CD56− | <0.5% |
| CD19+ | <10% |
| CD14+ | <10% |
| CD16+ | >10% |
| CD62L+ | >10% |

TABLE B
Phenotypic

| Parameter | % of nucleated cells | | | | | | | | |
|---|---|---|---|---|---|---|---|---|---|
| CD3⁺/CD56⁻ | 0.27 | 0.49 | 0.00 | 0.49 | 0.49 | 0.20 | 0.00 | 0.01 | 0.04 |
| % CD19+ | 0.32 | 0.09 | 0.10 | 0.09 | 0.07 | 0.02 | 0.10 | 0.10 | 0.09 |
| % CD14+ | 0.00 | 0.00 | 0.00 | 0.00 | 0.00 | 0.00 | 0.00 | 0.00 | 0.00 |
| % CD56⁺ | 98.24 | 98.18 | 99.78 | 98.18 | 97.66 | 98.24 | 99.78 | 99.68 | 99.64 |
| % CD16⁺ | 81.93 | * | 63.24 | * | 80.95 | 87.76 | 63.24 | 49.28 | 70.12 |
| % CD62L⁺ | 82.40 | 74.99 | 95.00 | 74.99 | 91.91 | 30.53 | 95.00 | 43.72 | 93.30 |

TABLE C
Phenotypic

| Parameter | % of nucleated cells | | | | | | | | | |
|---|---|---|---|---|---|---|---|---|---|---|
| CD3⁺/CD56⁻ | 0.12 | 0.06 | 0.23 | 0.19 | 0.22 | 0.33 | 0.48 | 0.01 | 0.01 | 0.01 |
| % CD19+ | 0.38 | 0.21 | 0.64 | 0.41 | 0.16 | 0.18 | 0.19 | 0.17 | 0.29 | 0.06 |
| % CD14+ | 0.00 | 0.00 | 0.00 | 0.00 | 0.00 | 0.00 | 0.04 | 0.00 | 0.00 | 0.01 |
| % CD56⁺ | 98.17 | 98.68 | 98.39 | 98.35 | 97.85 | 97.75 | 97.75 | 99.59 | 99.54 | 99.82 |
| % CD16⁺ | 41.14 | 30.02 | 53.65 | 66.38 | 62.07 | 39.18 | 53.83 | 20.7 | 36.03 | 61.43 |
| % CD62L⁺ | 31.9 | 37.74 | 56.07 | 61.20 | 61.1 | 13.94 | 54.65 | 32.62 | 53.35 | 81.35 |

TABLE D

| Phenotypic | | | LAG3 | | |
|---|---|---|---|---|---|
| Marker | CD200R | NKp80 | (CD223) | CD49a | CD57 |
| % of | 11.94 | 26.39 | 16.46 | 82.05 | 11.07 |
| nucleated | 10.92 | 76.56 | 0.57 | 75.35 | 12.66 |
| cells | 3.68 | 15.77 | 26.42 | 61.78 | 12.47 |
| | 1.03 | 19.57 | 4.8 | 69.64 | 3.59 |
| | 30.82 | 66.33 | 1.92 | 96.02 | 2.05 |
| | 15.88 | 43.83 | 1.45 | | 9.71 |
| | 21.07 | 62.93 | 11.21 | 80.2 | 17.25 |
| | 31.77 | 52.28 | 3.58 | 44.99 | 24.59 |
| | 22.9 | 69.95 | 7.56 | 65.25 | 17.54 |
| | 19.17 | 76.68 | 1.11 | 78.1 | 15.91 |
| | 31.81 | 71.13 | 19.24 | 52.38 | |
| | 19.65 | 58.13 | 8.63 | 68.9 | |
| | 13.03 | 27.55 | 26.89 | 79.56 | |
| | 13.37 | 58.8 | 5.72 | 68.42 | |

The present disclosure also provides a cryopreserved NK cell fraction, comprising any of the NK cell fractions described herein and DMSO. In some aspects, the concentration of DMSO can be about 1% v/v, or about 2% v/v, or about 3% v/v, or about 4% v/v, or about 5% v/v, or about 6% v/v, or about 7% v/v, or about 8% v/v, or about 9% v/v, or about 10% v/v, or about 11% v/v, or about 12% v/v, or about 13% v/v, or about 14% v/v, or about 15% v/v. In some aspects, the concentration of DMSO can be about 10% v/v.

In some aspects, a cryopreserved NK cell fraction can be stable for at least about six weeks, or at least about 1 month, or at least about 2 months, or at least about 3 months, or at least about 4 months, or at least about 5 months, or at least about 6 months, or at least about 7 months, or at least about 9 months, or at least about 10 months, or at least about 11 months, or at least about 12 months. In some aspects, a cryopreserved NK cell fraction can be stable at about −80° C. for at least about six weeks, about 1 month, or at least about 2 months, or at least about 3 months, or at least about 4 months, or at least about 5 months, or at least about 6 months, or at least about 7 months, or at least about 9 months, or at least about 10 months, or at least about 11 months, or at least about 12 months. In some aspects, a cryopreserved NK cell fraction can be stable at about −80° C. for at least about six weeks. In some aspects, a cryopreserved NK cell fraction can be stable at about −150° C. for at least about six weeks, about 1 month, or at least about 2 months, or at least about 3 months, or at least about 4 months, or at least about 5 months, or at least about 6 months, or at least about 7 months, or at least about 9 months, or at least about 10 months, or at least about 11 months, or at least about 12 months. In some aspects, a cryopreserved NK cell fraction can be stable at about −150° C. for at least about 12 months.

### Potency Assays of the Present Disclosure

The present disclosure provides a first potency assay, the assay comprising the steps of:
a) incubating an NK cell fraction of the present disclosure and a plurality of target cells, wherein the plurality of target cells is stained with at least one proliferation stain;
b) determining the cell death percentage in the plurality of target cells.

In some aspects of the first potency assay, the incubation conditions of step (a) can further comprise at least one anti-cancer therapeutic monoclonal antibody.

In some aspects of the first potency assay, the target cells can be K562 cells.

In some aspects of the first potency assay, the target cells can be Raji (CCL-86) cells.

In some aspects of the first potency assay, the target cells can be Raji (CCL-86) cells, and the incubation conditions of step (a) can further comprise rituximab. In some aspects, the rituximab can be present at a concentration of about 1 μg/ml.

As would be appreciated by the skilled artisan, determining the cell death percentage in the plurality of target cells in step (b) of the first potency assay can be accomplished using any standard technique known in the art for determining cell death percentages. In a non-limiting example, determining the cell death percentage in the plurality of target cells can comprise: i) staining the NK cell fraction and plurality of target cells incubated in step (a) with at least one viability stain; ii) using fluorescent activated cell sorting (FACS) to separate the plurality of target cells from the NK cell fraction; and iii) using the viability stain to determine the cell death percentage in the plurality of target cells sorted in separated in step (ii). In some aspects, using the viability stain to determining the cell death percentage in the plurality of target cells sorted in separated in step (ii) further comprises using the proliferation stain in combination with viability stain to determine the cell death percentage.

As would be appreciated by the skilled artisan, determining the cell death percentage in the plurality of target cells in step (b) of the first potency assay can be accomplished using any standard technique known in the art for determining cell death percentages. In a non-limiting example, determining the cell death percentage in the plurality of target cells can comprise: i) staining the NK cell fraction and plurality of target cells incubated in step (a) with at least one viability stain; ii) using a live-cell analysis system to detect the viability stain, thereby determining the cell death percentage in the plurality of target cells sorted in separated in step (ii). In some aspects, using the viability stain to determining the cell death percentage in the plurality of target cells sorted in separated in step (ii) further comprises using the proliferation stain in combination with viability stain to determine the cell death percentage.

In some aspects, the at least one proliferation stain can be carboxyfluorescein diacetate, succinimidyl ester (CFSE). As would be appreciated by the skilled artisan, any proliferation stain known in the art can be used in the first potency assay, described herein.

In some aspects, the at least one viability stain can be Helix NP™ Blue (also known as Sytox™ Blue). As would be appreciated by the skilled artisan, any proliferation stain known in the art can be used in the first potency assay.

In some aspects, and as would be appreciated by the skilled artisan, a live-cell analysis system can be an IncuCyte® system.

In some aspects, the incubation in step (a) of the first potency assay can be performed at about 37° C.

In some aspects, the incubation in step (a) of the first potency assay can be performed for at least about three hours.

In some aspects, the ratio of the number of cells in the NK cell fraction to the number of cells in the plurality of target cells in step (a) of the first potency assay can be about 2.5:1, or about 5:1, or about 10:1.

In some aspects, an NK cell fraction of the present disclosure can be characterized in that when the NK cell fraction is tested using first potency assay described above, wherein target cells are K562 cells, the cell death percentage in the target cells is at least about 10%. In some aspects, an NK cell fraction of the present disclosure can be characterized in that when the NK cell fraction is tested using first potency assay described above, wherein target cells are K562 cells, the cell death percentage in the target cells is at least about 50%, or at least about 60%, or at least about 70%, or at least about 80%, or at least about 90%.

In some aspects, an NK cell fraction of the present disclosure can be characterized in that when the NK cell fraction is tested using first potency assay described above, wherein target cells are K562 cells, wherein the ratio of the number of cells in the NK cell fraction to the number of cells in the plurality of target cells in step (a) of the first potency assay is about 5:1, the cell death percentage in the target cells is at least about 10%. In some aspects, the cell death percentage is measured after about 12 hours of incubation of the NK cell fraction with the target cells.

In some aspects, an NK cell fraction of the present disclosure can be characterized in that when the NK cell fraction is tested using first potency assay described above, wherein target cells are K562 cells, wherein the ratio of the number of cells in the NK cell fraction to the number of cells in the plurality of target cells in step (a) of the first potency assay is about 10:1, the cell death percentage in the target cells is at least about 10%. In some aspects, the cell death percentage is measured after about 3 hours of incubation of the NK cell fraction with the target cells.

In some aspects, an NK cell fraction of the present disclosure can be characterized in that when the NK cell fraction is tested using first potency assay described above, wherein target cells are Raji cells, the cell death percentage in the target cells is at least about 10%, or at leaset about 20%, or at least about 30%, or at least about 40%, at least about 50%, or at least about 60%, or at least about 70%, or at least about 80%, or at least about 90%.

In some aspects, an NK cell fraction of the present disclosure can be characterized in that when the NK cell fraction is tested using first potency assay described above, wherein target cells are Raji cells and the incubation conditions of step (a) further comprise rituximab, the cell death percentage in the target cells is at least about 10%, or at leaset about 20%, or at least about 30%, or at least about 40%, at least about 50%, or at least about 60%, or at least about 70%, or at least about 80%, or at least about 90%.

In some aspects, an NK cell fraction of the present disclosure can be characterized in that when the NK cell fraction is tested using: first potency assay described above, wherein target cells are Raji cells; and the first potency assay described above, wherein the target cells are Raji cells and the incubation conditions of step (a) further comprise rituximab, the cell death percentage in the target cells without the rituximab is less than the cell death percentage in the target cells when rituximab is present.

The present disclosure provides a second potency assay, the assay comprising the steps of:
a) incubating an NK cell fraction of the present disclosure and a plurality of target cells, wherein the NK cell fraction is stained with at least one anti-CD107α antibody comprising a detectable label;
b) treating the NK cell fraction and the plurality of target cells incubated in step (a) with one or more protein trafficking inhibitors and further incubating the NK cell fraction and the plurality of target cells;
c) staining the NK cell fraction and plurality of target cells with:
   at least one viability stain;
   at least one anti-CD56 antibody comprising a detectable label
d) fixing the NK cell fraction and the plurality of target cells;
e) permeabilizing the NK cell fraction and the plurality of target cells;
f) staining the NK cell fraction and plurality of target cells with:
   i) at least one anti-IFNγ antibody comprising a detectable label;
   ii) at least one anti-TNFα antibody comprising a detectable label;
g) determining at least one of:
   g₁) the percentage of viable cells stained with the at least one anti-CD56 antibody that are also stained with the at least one anti-CD107α antibody (i.e. number of CD107a+/CD56+ cells÷number of CD56+ cells×100%);
   g₂) the percentage of viable cells stained with the at least one anti-CD56 antibody that are also stained with the at least one anti-IFNγ antibody (i.e. number of IFNγ+/CD56+ cells÷number of CD56+ cells×100%); and
   g₃) the percentage of viable cells stained with the at least one anti-CD56 antibody that are also stained with the at least one anti-TNFα antibody (i.e. number of TNFα+/CD56+ cells÷number of CD56+ cells×100%).

In some aspects of the second potency assay, the target cells can be K562 cells.

In some aspects of the second potency assay, the target cells can be Raji (CCL-86) cells.

In some aspects of the second potency assay, the target cells can be Raji (CCL-86) cells, and the incubation conditions of step (a) and step (b) can further comprise rituximab. In some aspects, the rituximab can be present at a concentration of about 0.5 μg/ml.

In some aspects, the at least one viability stain can be Zombie Violet™ Viability Dye. As would be appreciated by the skilled artisan, any proliferation stain known in the art can be used in the first potency assay.

In some aspects, the one or more protein trafficking inhibitors can comprise brefeldin, GolgiStop™ Protein Transport Inhibitor (BD), a combination of brefeldin and GolgiStop™ Protein Transport Inhibitor, or any other protein tracking inhibitors known in the art.

In some aspects of the second potency assay, the further incubation in step (b) is performed at about 37° C.

In some aspects of the second potency assay, the further incubation in step (b) is performed for at least about 37° C.

As would be appreciated by the skilled artisan, determining at least one (g₁)-(g₃) of step (g) can be accomplished using any standard technique known in the art for determining percentages of cells labeled with antibodies comprising detectable labels, including, but not limited to fluorescent activated cell sorting (FACS).

In some aspects of the second potency assay, step (g) can comprise determining each of (g₁)-(g₃).

In some aspects of the second potency assay, the ratio of the number of cells in the NK cell fraction to the number of cells in the plurality of target cells in step (a) of the second potency assay can be about 1:3.

In some aspects, an NK cell fraction of the present disclosure can be characterized in that when the NK cell fraction is tested using second potency assay described above, wherein target cells are K562 cells, the percentage of viable cells stained with the at least one anti-CD56 antibody that are also stained with the at least one anti-CD107α antibody is at least 10%.

In some aspects, an NK cell fraction of the present disclosure can be characterized in that when the NK cell fraction is tested using first potency assay described above, wherein target cells are Raji cells, the percentage of viable cells stained with the at least one anti-CD56 antibody that are also stained with the at least one anti-CD107α antibody is at least 4%.

In some aspects, an NK cell fraction of the present disclosure can be characterized in that when the NK cell fraction is tested using first potency assay described above, wherein target cells are K562 cells, the percentage of viable cells stained with the at least one anti-CD56 antibody that are also stained with the at least one anti-IFNγ antibody is at least 10%.

In some aspects, an NK cell fraction of the present disclosure can be characterized in that when the NK cell fraction is tested using first potency assay described above, wherein target cells are K562 cells, the percentage of viable cells stained with the at least one anti-CD56 antibody that are also stained with the at least one anti-TNFα antibody is at least 10%.

Thus, according to one aspect of an embodiment of the present invention there is provided a method of preparing an expanded NK cell fraction for a subject in need thereof, the method comprising:
(a) obtaining an apheresis product comprising NK cells and CD3+ cells allogeneic to subject;
(b) separating the apheresis product into a CD3-depleted cell fraction and a CD3+ cell fraction;
(c) inactivating cells of said CD3+ cell fraction by irradiation;
(d) ex vivo culturing said CD3-depleted cell fraction with the inactivated, irradiated CD3+ cell fraction under conditions allowing for cell proliferation, wherein said conditions comprise providing nutrients, serum, IL-15, a CD3 agonist and nicotinamide in an amount between 1.0 mM to 10 mM;
(e) supplementing said combined CD3-depleted and CD3+ cell fractions with fresh nutrients, serum, IL-15 and nicotinamide 6-10 days following step (d) to produce an expanded CD3-depleted cell fraction;
(f) harvesting said combined CD3-depleted and CD3+ cell fractions 14-16 days following step (d); and
(g) washing and concentrating said combined CD3-depleted and CD3+ cell fraction of step (f), thereby producing an expanded NK cell fraction for administration to said subject.

As used herein, the term natural killer (NK) cells refers to large granular lymphocytes involved in the innate immune response. Functionally, NK cells exhibit cytolytic activity against a variety of targets via exocytosis of cytoplasmic granules containing a variety of proteins, including perforin, and granzyme proteases. Killing is triggered in a contact-dependent, non-phagocytotic process which does not require prior sensitization to an antigen. Human NK cells are characterized by the presence of the cell-surface markers CD16 and CD56, and the absence of the T cell receptor (CD3). Human bone marrow-derived NK cells are further characterized by the CD2+CD16+CD56+CD3− phenotype, further containing the T-cell receptor zeta-chain [zeta(ζ)-TCR], and often characterized by NKp46, NKp30 or NKp44. Non-NK cells such as NKT cells or CD8NKT possess characteristics and cell-surface markers of both T cells and NK cells. In one embodiment, the method of the present invention is employed for ex-vivo propagation of mature NK cells from a population of cells. As used herein, the term “mature NK cell” is defined as a committed NK cell, having characteristic surface markers and NK cell function, and lacking the potential for further differentiation. As use herein, mature NK cells include, but are not limited to CD56^{bright} cells, which can proliferate and produce abundant cytokines, CD56^{dim} cells, exhibiting robust cytotoxicity, CD56^{bright}CD94^{high} and CD56^{dim}CD94^{high} cells. In another embodiment, NK progenitor cells, or mixed populations of NK progenitor cells and mature NK cells are propagated. Cell surface expression of the CD56, CD3, CD94 and other markers can be determined, for example, via FACS analysis or immunohistological staining techniques.

As used herein, the term “progenitor” refers to an immature cell capable of dividing and/or undergoing differentiation into one or more mature effector cells. Lymphocyte progenitors include, for example, pluripotent hematopoietic stem cells capable of giving rise to mature cells of the B cell, T cell and NK lineages. In the B cell lineage (that is, in the developmental pathway that gives rise to mature B cells), progenitor cells also include pro-B cells and pre-B cells characterized by immunoglobulin gene rearrangement and expression. In the T and NK cell lineages, progenitor cells also include bone-marrow derived biopotential T/NK cell progenitors [e.g., CD34(+)CD45RA(hi)CD7(+) and CD34(+)CD45RA(hi)Lin(−)CD10(+) cells], as well as intrathymic progenitor cells, including double negative (with respect to CD4 and CD8) and double positive thymocytes (T cell lineage) and committed NK cell progenitors.

NK cells of the present invention may be derived from any source which comprises such cells. NK cells are found in many tissues, and can be obtained, for example, from lymph nodes, spleen, liver, lungs, intestines, deciduas and can also be obtained from iPS cells or embryonic stem cells (ESC). Typically, cord blood, peripheral blood, mobilized peripheral blood and bone marrow, which contain heterogeneous lymphocyte cell populations, are used to provide large numbers of NK cells for research and clinical use. In specific embodiments, the NK cells of the present invention are an NK cell fraction of an apheresis product or leukapheresis product.

In apheresis, whole donor blood is separated into blood components (e.g. plasma, leukocytes and erythrocytes), typically by centrifugation, selected components are drawn off for manipulation (e.g. culturing of leukocyte fractions) and the remainder is returned to the donor. Apheresis has the advantage of providing specific blood fractions (for example, leukocyte fraction) in large numbers without depleting fluids (e.g. plasma) and other blood components. Apheresis can be based on continuous flow centrifugation, which requires a low extracorporeal volume, or based on intermittent flow centrifugation of the blood, which separates the components in cycles, but is typically more time consuming and characterized by larger extracorporeal volumes of the donor's blood. Many suitable apheresis devices are commercially available. Typically, apheresis applies to separation of blood components from the peripheral blood of the donor.

As used herein, the term “apheresis product” refers to cells collected from the circulating blood of a donor by apheresis. Clinical experience with NK cell infusions has shown that allogeneic NK cells can successfully engraft in hosts, with a lower incidence of graft versus host disease (GVHD). An “apheresis product allogeneic to the subject”, as used herein, refers to cells collected from a donor other than the subject himself. In specific embodiments, the apheresis product or products are apheresis products allogeneic to the subject. In some embodiments, specified donor or donors may be identified, to provide apheresis products for a known subject. In other embodiments, the allogeneic apheresis product is selected from a “bank” of suitable apheresis products without consideration of the identity of the donor.

The apheresis product, in some aspects, contains lymphocytes, including T cells, monocytes, granulocytes, B cells, other nucleated white blood cells, red blood cells, and/or platelets, and in some aspects contains cells other than red blood cells and platelets. A variety of apheresis methodologies are known in the art, for example those disclosed in U.S. Patent Publication Nos. 20160184361, 20180043082, 20170021083, 20060116271, 20050155932, 20050143684 and 20030195455, the contents of which are incorporated by reference.

In some embodiments, the cells collected from the donor are washed, e.g., to remove the plasma fraction and to place the cells in an appropriate buffer or media for subsequent processing steps. In some embodiments, the cells are washed with phosphate buffered saline (PBS) and resuspended in PBS. In some embodiments, the cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components. In some embodiments, the methods include lysing the red blood cells with an RBC lysing buffer (e.g. Ammonium-Chloride-Potassium (ACK) buffer), repeated washing and centrifugation until clear or light pink.

Thus, according to one aspect of one embodiment of the present invention, the method comprises culturing a CD3-depleted cell fraction comprising NK cells, wherein the CD3− depleted cell fraction is from apheresis. In specific embodiments, the CD3-depleted cell fraction is from apheresis units obtained from donors using a PCS2 or MCS8150 Haemonetics apheresis machine (Haemonetics, Boston, MA). In certain embodiments, the CD3-depleted cell fraction is from apheresis units obtained from peripheral blood of the donor. Prior to the apheresis, donors may be treated with compositions (e.g. Plerixafor) known to mobilize desired cell populations (e.g. lymphocytes) from hematopoietic cell sources, such as bone marrow and the spleen.

It will be appreciated, that due to the fact that the expanded NK cell populations are intended for use in the clinical setting (e.g. infusion into a patient), the apheresis products chosen for cell selection and expansion according to the methods described herein, are clinical grade apheresis units.

In some embodiments the apheresis products can be fresh cell populations, while in other embodiments the cells are cultured from stored apheresis cell populations (such as cryopreserved and thawed cells). In other embodiments, the cells are cultured from previously cultured cell populations.

In some embodiments, the apheresis product is evaluated for viability and cell phenotype prior to separation of CD3− and CD3+ cells. In specific embodiments, cells of the apheresis product are at least 70, 80, 85, 90, 91, 92, 93, 94, 95, 96, 97, 98, 99 or 100% viable. In some embodiments, cell viability is assessed by FACS and/or Cedex (automated Trypan Blue exclusion assay). In specific embodiments, the cells of the apheresis product are greater than 90% viable.

It will be appreciated that evaluation for total cell number, viability, phenotype, potency and/or function can be carried out on any of the cell populations at any point in the process- for example, pre- or post CD3 depletion, pre-expansion, seeding, at any time during culturing, harvest, pre-cryopreservation, post cryopreservation, during storage, pre-thawing, during and/or post-thawing, pre- or during infusion. The resultant profiles (cell number, viability, phenotype and/or function) can be used for monitoring the cell populations, or may be used to identify selection or release criteria—e.g. for selection or exclusion of cell populations or preparations at any point in the claimed methods, or for modifying protocol (e.g. number of cells in a unit may determine the need for combination of units to proceed to the next step in the protocol).

Relevant parameters that can be evaluated for the claimed method include number of cells, cell viability (e.g. Cedex, see above), contamination (e.g. mycoplasma, bacteria, endotoxin levels), sterility, NK-related cell functions (e.g. cell killing assay, antibody dependent cellular cytotoxicity-ADCC, engraftment potential), phenotype (e.g. cellular markers such as CD56, CD3, CD16, CD107a, CD62L), potency (CD107a and CD62L, cytokine secretion—e.g. INF-γ, TNF-α, GM-CSF), appearance and temperature. In specific embodiments, cell populations prior to, during, and/or following processing in any of the steps of the methods of the invention can be evaluated for potency, including viability (e.g. using Zombie Violet Viability Dye (Biolegend)), for surface markers CD107a, CD56 and CD16 and for INF-γ, TNF-α by flow cytometry (FACS) analysis or ELISA with specific antibodies, and for NK function (ADCC and cell killing).

Lymphocyte fractions, such as “buffy coat” or apheresis units can be processed to enrich or purify or isolate specific defined populations of cells. The terms “purify” and “isolate” do not require absolute purity; rather, these are intended as relative terms. Thus, for example, a purified lymphocyte population is one in which the specified cells are more enriched than such cells are in its source tissue. A preparation of substantially pure lymphocytes can be enriched such that the desired cells represent at least 50% of the total cells present in the preparation. In certain embodiments, a substantially pure population of cells represents at least 60%, at least 70%, at least 80%, at least 85%, at least 90%, or at least 95% or more of the total cells in the preparation. In some embodiments, the apheresis product contains both CD3− (e.g. CD3-CD56+ NK cells) and CD3+ cells (e.g. T-cells and NKT cells).

Methods for enriching and isolating lymphocytes are well known in the art, and appropriate methods can be selected based on the desired population. For example, in one approach, the source material is enriched for lymphocytes by removing red blood cells. Based on density red blood cells are separated from lymphocytes and other cells. The lymphocyte rich fractions can then be selectively recovered. Lymphocytes and their progenitors can also be enriched by centrifugation using separation mediums such as standard Lymphocyte Separation Medium (LSM) available from a variety of commercial sources. Alternatively, lymphocytes/progenitors can be enriched using various affinity based procedures. Numerous antibody mediated affinity preparation methods are known in the art such as antibody conjugated magnetic beads. Lymphocyte enrichment can also be performed using commercially available preparations for negatively selecting unwanted cells, such as FICOLL-HYPAQUE™ and other density gradient mediums formulated for the enrichment of whole lymphocytes, T cells or NK cells.

In some embodiments of the invention, the apheresis product is separated into a CD3− depleted and a CD3-containing (CD3+) cell fraction. Methods of selection of CD3− cells from blood, bone marrow, lymphocyte preparations (e.g. apheresis units) or tissue samples are well known in the art. Most commonly used are protocols based on mononuclear cell fractionation, and depletion of non-NK cells such as CD3+. Depletion of other cell types, such as CD34+ and CD133+ may also be performed. Combinations of two or more protocols can be employed to provide CD3-depleted cell populations having greater purity from non-NK contaminants. Commercially available kits for isolation of CD3-negative cells include one-step procedures and multistep procedures, including depletion, or partial depletion, of CD3+ or depletion with non-NK cell antibodies recognizing and removing T cells. In specific embodiments, the NK cell fraction is depleted of CD3 cells by immunomagnetic selection, for example, using a CliniMACS T cell depletion set ((LS Depletion set (261-01) Miltenyi Biotec, using CliniMACS CD3 reagent (Miltenyi REF 273-01, Miltenyi Biotech). In specific embodiments, the CD3− depleted cell fraction and the CD3+ cell fraction are separately washed and centrifuged.

In some embodiments, the CD3-depleted fraction comprises CD56+CD16+CD3− cells and or CD56+CD16-CD3− cells. In specific embodiments, the CD3-depleted fraction comprises at least 10% CD56+/CD3− cells, at least 20% CD56+/CD3− cells, at least 30% CD56+/CD3− cells, at least 40% CD56+/CD3− cells, at least 50% CD56+/CD3− cells, at least 60% CD56+/CD3− cells, at least 70% CD56+/CD3− cells, at least 80% CD56+/CD3− cells or at least 90% CD56+/CD3− cells. In some embodiments, the CD3-depleted fraction comprises between 40%-97% CD56+/CD3− cells, between 30%-90% CD56+/CD3− cells, between 40%-80% CD56+/CD3− cells, between 55-75% CD56+/CD3− cells, between 60%-70% CD56+/CD3− cells. In some embodiments, the CD3-depleted fraction comprises between 15% and 90% CD56+/CD3− cells. In some embodiments, the CD3-depleted fraction comprises fewer than 5%, fewer than 2%, fewer than 1%, fewer than 0.8%, fewer than 0.5%, fewer than 0.25%, fewer than 0.1%, fewer than 0.05%, fewer than 0.025% CD3+ cells.

Methods for selection of cells according to phenotype include, but not exclusively, immunodetection (e.g. ELISA) and FACS analysis.

In some embodiments, the CD3-depleted cell fractions are expanded by adding feeder cells to the culture, such as non-dividing feeder cells. Suitable feeder cells include, but not exclusively, T-cells, stromal cells, peripheral blood mononuclear cells (PBMCs), fibroblasts and the like, irradiated or otherwise inactivated to prevent their proliferation in culture while maintaining their contribution to NK cell culture conditions. In specific embodiments, the CD3+ cell fraction of the apheresis product, following separation, is inactivated to provide feeder cells for the culture of the CD3-depleted cells. In some aspects, the non-dividing feeder cells can comprise gamma- or X-ray-irradiated CD3+ feeder cells. Irradiation can be accomplished using any of the commercially available cell irradiation devices, such as the CELLRAD irradiator (Precision X-Ray, N Branford CT). In some embodiments, the CD3+ cell fraction cells are irradiated with X-rays in the range of about 30 to 50 Gy, 130 kV to prevent cell division. In specific embodiments, the CD3+ fraction is irradiated using a CELLRAD irradiator at a target dosage of 40 Gy, 130 kV, 5 mA. Following irradiation, the CD3+ cell fraction can be washed, concentrated by centrifugation and resuspended, for example, in culture medium in advance of seeding. Inactivation of the CD3+ cells can be evaluated by providing the cells with conditions for culture (medium, temperature and moisture) and monitoring growth or the absence thereof.

The present inventors have uncovered that culture of the CD3− cell faction, comprising NK cells along with non-proliferating feeder cells provides greater expansion and enhanced function of the expanded NK cell population. Thus, in some aspects, the CD3− cell fraction comprising NK cells is seeded for culture along with inactivated (e.g. irradiated) feeder cells. In specific embodiments, the CD3− cell fraction comprising NK cells is seeded for culture with irradiated CD3+ cells. In a particular embodiment, the CD3− cell fraction comprising NK cells is seeded for culture along with the corresponding CD3+ cell fraction from the same apheresis product.

In some embodiments, the CD3− cells and the CD3+ cells are seeded for culture at a (numerical) ratio in the range of 0.1:1 to 5:1 CD3− cells to CD3+ cells. In some embodiments, the CD3− to CD3+ cell ratio for seeding is 0.2:1, 0.5:1, 0.75:1, 0.8:1, 0.9:1, 1:1, 1.1:1, 1.2:1, 1.4:1, 1.8:1, 2:1, 2.5:1, 3:1, 4:1 or 5:1. In particular embodiments, the CD3+ cells are seeded at a ratio of 1:1 with cells from the CD3− fraction.

In some aspects of the invention, the cells are seeded in the range of 0.20-0.60×10⁶ cells/ml. In some embodiments, the cells are seeded at a concentration of 0.25×10⁶ cells/ml, 0.30×10⁶ cells/ml, 0.35×10⁶ cells/ml, 0.40×10⁶ cells/ml, 0.45×10⁶ cells/ml, 0.50×10⁶ cells/ml, 0.55×10⁶ cells/ml, or 0.60×10⁶ cells/ml. In specific embodiments, the cells are seeded at a concentration of 0.30-0.40×10⁶ cells/ml, in particular, 0.35×10⁶ cells/ml. In specific embodiments, seeding of cells for culture is affected at 0.35×10⁶ CD3-depleted and 0.35×10⁶ irradiated CD3+ cells/ml.

The cells for culture can be seeded in cell culture flasks, or other suitable vessels. In some embodiments, the cells are seeded in gas-permeable cell culture flasks, such as G-Rex flasks (WilsonWolf, St Paul MN), in particular, G-Rex flasks. Flask volume can be selected according to need—in specific embodiments, the cells are seeded into G-Rex500MCS flasks, GMP grade.

In some embodiments, the entire volume of the CD3-depleted and the CD3+ cell fractions (obtained from the separation of the apheresis unit) are seeded for culture. Thus, in some embodiments, seeding into more than a single flask is required. For example, when seeding of cells for culture is affected at 0.35×10⁶ CD3-depleted and 0.35×10⁶ irradiated CD3+ cells/ml, using flask with 2 liters volume of medium, if the numbers of cells in the CD3-depleted and/or CD3+ cell fractions exceeds 700×10⁶ cells per fraction, additional flasks will be needed to seed the entire volume of the CD3-depleted and the CD3+ cell fractions. Thus, in some embodiments, culturing of the cells is affected in flasks at 400-900×10⁶ CD3-depleted and 400-900×10⁶ irradiated CD3+ cells per flask, in particular, at 700×10⁶ CD3-depleted and 700×10⁶ irradiated CD3+ cells per flask.

NK cells can be cultured ex-vivo by short- or long-term culture. The present inventors have demonstrated that NK cells can be cultured with growth factors and nicotinamide and/or other nicotinamide moiety, for as little as 7 days, or as many as 3 weeks resulted in enhanced, preferential proliferation and/or functionality of the cultured NK cells, as compared to cells cultured with cytokines but with less than 0.1 mM nicotinamide and/or other nicotinamide moiety (see PCT Publication WO2011/080740). In clinical trials, infusion of freshly prepared, haploidentical NK cells expanded from a positive-selected CD56+/CD3− cell fraction with nicotinamide for 14-16 days into NHL and MM patients resulted in efficient engraftment and a significant proportion of progression free survival (see Bachanova et al. 2019, Blood, Volume 134, Supplement 1, Page 777). However, since use of freshly expanded cells is severely limiting, it is desirable to provide greater ex-vivo NK cell expansion while retaining therapeutically advantageous functionality of the expanded NK cell fractions, suitable for cryopreservation and storage.

Thus, in specific embodiments, the CD3-depleted cell and CD3+ cell fractions are cultured over a period of 14-16 days.

Ex-vivo culturing of the CD3-depleted fraction comprising the NK cells can be effected, according to this aspect of the present invention, by providing the CD3-depleted cells ex vivo with conditions for cell proliferation and ex vivo culturing the CD3-depleted cells with a nicotinamide moiety, thereby ex-vivo expanding the population of NK cells.

It will be appreciated that culturing of the CD3-depleted fraction, when combined with the irradiated CD3+ fraction, is directed to proliferation and functional enhancement of the NK cell fraction, and that the inactivated CD3+ fraction is non-proliferating and does not undergo significant expansion under the culture conditions of the methods described herein.

As used herein “culturing” includes providing the chemical and physical conditions (e.g., temperature, gas) which are required for NK cell maintenance, and growth factors. In one embodiment, culturing the combined CD3-depleted cell and CD3+ cell fractions includes providing the cells with conditions for NK cell proliferation. Examples of chemical conditions which may support NK cell proliferation include but are not limited to buffers, nutrients, serum, vitamins and antibiotics as well as cytokines and other growth factors which are typically provided in the growth (i.e., culture) medium. In a particular embodiment, conditions for cell proliferation comprise nutrients, serum and cytokine(s). In one embodiment, the NK culture medium includes a minimal essential medium (MEM), such as MEMα (BI, Bet HaEmek, Israel) and serum. In some embodiments, the serum is provided at 2-20%, 5-15% or 5-10% of the culture medium. In specific embodiments, the serum is human serum, provided at 10% of the culture medium. In a particular embodiment, the culture medium is MEMα comprising 10% Human AB Serum (Sigma-Aldrich, St. Louis, MO). Other media suitable for use with the invention include, but are not limited to Glascow's medium (Gibco Carlsbad CA), RPMI medium (Sigma-Aldrich, St Louis MO) or DMEM (Sigma-Aldrich, St Louis MO). It will be noted that many of the culture media contain nicotinamide as a vitamin supplement for example, MEMα (8.19 μM nicotinamide), RPMI (8.19 μM nicotinamide), DMEM (32.78 μM nicotinamide) and Glascow's medium (16.39 μM nicotinamide), however, the methods of the present invention relate to exogenously added nicotinamide supplementing any nicotinamide and/or nicotinamide moiety included the medium's formula, or that resulting from overall adjustment of medium component concentrations.

According to some embodiments of the present invention, culturing the CD3-depleted cell and CD3+ cell fractions under conditions allowing for NK cell proliferation comprises providing the cells with nutrients, serum and cytokines. In some embodiments the at least one growth factor includes cytokines and/or chemokines. Cytokines and other growth factors are typically provided in concentrations ranging from 0.5-100 ng/ml, or 1.0-80 ng/ml, more typically 5-750 ng/ml, yet more typically 5.0-50 ng/ml (up to 10× such concentrations may be contemplated), and are available commercially, for example, from Perpo Tech, Inc., Rocky Hill, NJ, USA. In one embodiment, conditions allowing for cell proliferation includes providing the cytokine interleukin 15 (IL-15). In specific embodiments, the CD3-depleted cells are cultured with 20 ng/ml IL-15.

Further conditions for NK cell proliferation in culture include provision of Glutamine, as an auxiliary source of energy and nitrogen. In some embodiments the culture medium comprises 0.5-5 mM Glutamine. In particular embodiments, the culture medium comprises 2 mM Glutamine. Glutamine may be provided as Glutamine or as a dipeptide L-alanine-L-glutamine for greater stability in culture.

The culture medium typically also comprises antibiotics (e.g. gentamicin, penicillin, streptomycin); in some embodiments, the culture medium comprises gentamicin in the range of 0.01-1.0 mg/ml. In particular embodiments, gentamicin is provided in the culture medium at 0.05 mg/ml.

In cells cultured with non-proliferating (e.g. irradiated) CD3+ cells, stimulation of the T-cells to secrete growth factors beneficial to the CD3-depleted cell fraction is desired. Thus, in some embodiments, the CD3-depleted cell and CD3+ cell fractions are seeded in culture medium supplemented with a CD3 agonist. CD3 agonists suitable for use with the method of the invention include, inter alia, anti-CD3 monoclonal-CD3 agonist antibodies such as OKT-3 (also known as Muromonab-CD3), mAb 145-2C11, MGA031 and ChAglyCD3. In specific embodiments, the CD3 agonist OKT-3 is provided, at seeding, at a range of concentrations between 0.1 and 5 μg/ml. In particular embodiments, OKT-3 (available as MACS® GMP CD3 pure, Milentyi 170-076-116) is provided in the culture medium at seeding at a concentration of 1 μg/ml.

Further, it will be appreciated in this respect that novel cytokines are continuously discovered, some of which may find uses in the methods of NK cell proliferation of the present invention.

According to one embodiment, the CD3-depleted cell and CD3+ cell fractions are cultured with nutrients, serum, a cytokine (e.g. IL-15), a CD3 agonist and nicotinamide and/or a nicotinamide moiety. As used herein, the term “nicotinamide moiety” refers to nicotinamide as well as to products that are derived from nicotinamide, derivatives, analogs and metabolites thereof, such as, for example, NAD, NADH and NADPH, which are capable of effectively and preferentially enhancing NK cell proliferation and/or activation. Nicotinamide derivatives, analogs and metabolites can be screened and evaluated for their effect on ex-vivo NK proliferation in culture by addition to NK cultures maintained as described hereinbelow, addition to functional assays such as killing and motility assays, or in automated screening protocols designed for high-throughput assays well known in the art.

As used herein, the phrase “nicotinamide analog” refers to any molecule that is known to act similarly to nicotinamide in the abovementioned or similar assays. Representative examples of nicotinamide analogs can include, without limitation, benzamide, nicotinethioamide (the thiol analog of nicotinamide), nicotinic acid and α-amino-3-indolepropionic acid.

The phrase “nicotinamide derivative” further refers to any structural derivative of nicotinamide itself or of an analog of nicotinamide. Examples of such derivatives include, without limitation, substituted benzamides, substituted nicotinamides and nicotinethioamides and N-substituted nicotinamides and nicotinthioamides, 3-acetylpiridine and sodium nicotinate. In one particular embodiment of the invention the nicotinamide moiety is nicotinamide.

Nicotinamide or nicotinamide moiety concentrations suitable for use in some embodiments of the present invention are typically in the range of about 0.5 mM to about 50 mM, about 1.0 mM to about 25 mM, about 1.0 mM to about 25 mM, about 2.5 mM to about 10 mM, about 5.0 mM to about 10 mM. Exemplary effective concentrations of nicotinamide can be of about 0.5 to about 15 mM, 1.0-10.0 mM, typically 2.5 or 5.0 mM, based on the effect of these concentrations of nicotinamide on proliferation and NK cell function. According to some embodiments of the invention, nicotinamide is provided at a concentration in the range (mM) of about 0.5, about 0.75, about 1.0, about 1.25, about 1.5, about 1.75, about 2.0, about 2.25, about 2.5, about 2.75, about 3.0, about 3.25, about 3.5, about 3.75, about 4.0, about 4.25, about 4.5, about 4.75, about 5.0, about 5.25, about 5.5, about 5.75, about 6.0, about 6.25, about 6.5, about 6.75, about 7.0, about 7.25, about 7.5, about 7.75, about 8.0, about 8.25, about 8.5, about 8.75, about 9.0, about 9.25, about 9.5, about 9.75, about 10.0, about 11.0, about 12.0, about 13.0, about 14.0, about 15.0, about 16.0, about 17.0, about 18.0 and about 20.0 mM. All effective intermediate concentrations are contemplated. In specific embodiments, conditions allowing proliferation comprise between 1.0 to 10.0 mM nicotinamide. In yet other embodiments, conditions allowing proliferation comprise 7.0 mM nicotinamide.

Suitable concentrations of the nicotinamide and/or nicotinamide moiety can be determined according to any assay of NK proliferation, cell phenotype and/or activity, for example, cell culture or function (e.g. potency). Suitable concentration of nicotinamide is a concentration which use thereof in culture “enhances”, or results in a net increase of proliferation, specific phenotype and/or function of NK cells in culture, compared to “control” cultures having less than 0.1 mM of the nicotinamide and tested from the same NK cell source (e.g. cord blood, bone marrow or peripheral blood preparation), in the same assay and under similar culture conditions (duration of exposure to nicotinamide, time of exposure to nicotinamide).

In some studies, ex-vivo expansion of NK cells by culture with nutrients, serum, cytokines and nicotinamide does not require replenishing the medium or manipulation over the culture period, while other studies have advocated culture medium replenishment (“re-feeding”) at different intervals during the NK cell culture. In certain embodiments of the present invention, the CD3-depleted cell fraction is “re-fed” during the culture period. Thus, in specific embodiments, culturing the CD3-depleted cells comprises supplementing the CD3-depleted and CD3+ cell fractions with fresh nutrients, serum, IL-15 and nicotinamide 6-10 days following initiation of the ex-vivo culture (see step “e”). In some embodiments, supplementing is provided between 6-7 days following initiation of the ex-vivo culture, between 7-8 days following initiation of the ex-vivo culture, between 8-9 days following initiation of the ex-vivo culture, between 9-10 days following initiation of the ex-vivo culture (seeding) or between 6-10 days following initiation of culturing of the CD3-depleted cells. In some embodiments, supplementing (or “refeeding”) comprises removing about 30-80%, about 40-70% or about 45-55% of the medium of the cell culture, and replacing that with a similar (e.g. equivalent) volume of fresh medium having the same composition and level of nutrients, serum, cytokines (e.g. IL-15) and nicotinamide as the removed medium. In some embodiments, supplementing (or “refeeding”) comprises removing about 50% of the medium of the NK cell fraction culture, and replacing the removed medium with a similar (e.g. equivalent) volume of fresh medium having the same composition and level of nutrients, serum, cytokines (e.g. IL-15) and nicotinamide. In specific embodiments, no culture medium is removed for refeeding, and fresh culture medium comprising the same composition and level of nutrients, serum, cytokines (e.g. IL-15), antibiotics, glutamine and nicotinamide is added until the culture volume reaches approximately twice the original culture volume at initiation of the CD3-depleted cell culture (“seeding”). In particular embodiments, culture medium for “refeeding” does not contain a CD3-receptor agonist. (e.g. devoid of OKT-3).

NK cell populations can be cultured using a variety of methods and devices. Selection of culture apparatus is usually based on the scale and purpose of the culture. Scaling up of cell culture preferably involves the use of dedicated devices. Apparatus for large scale, clinical grade NK cell production is detailed, for example, in Spanholtz et al. (PLoS ONE 2010; 5:e9221) and Sutlu et al. (Cytotherapy 2010, Cytotherapy 12(8):1044-55). In some embodiments, culturing the NK cell fractions (e.g. steps (b) and/or (c) of the method) is effected in flasks, at a cell density of 100-4000×10⁶ cells per flask. In specific embodiments, culturing the NK cell fractions (e.g. initiation of the ex-vivo culture and/or “re-feeding”) is effected in flasks, at a cell density of 200-300×10⁶ cells per flask. In certain embodiments, the flasks are flasks comprising a gas-permeable membrane, such as the G-Rex culture device (G-Rex 100M or closed system G-Rex MCS, WolfWilson, St Paul MN).

It will be appreciated that the density of cells in the culture flask increases with proliferation of the cells over the duration of the culture. Thus, although culturing of the cells is affected in flasks by seeding at 400-900×10⁶ CD3-depleted and 400-900×10⁶ irradiated CD3+ cells per flask, in particular, at 700×10⁶ CD3-depleted and 700×10⁶ irradiated CD3+ cells per flask, over the course of expansion in culture, the cells of the CD3-depleted fraction are cultured at a cell density of 100-4000×10⁶ cells per flask, 150-3500×10⁶ cells per flask, 200-4000×10⁶ cells per flask, 300-4000×10⁶ cells per flask, 200-3000×10⁶ cells per flask, 300-2000×10⁶ cells per flask, 400-1000×10⁶ cells per flask, 250-800×10⁶ cells per flask, 100-600×10⁶ cells per flask or 150-500×10⁶ cells per flask. In specific embodiments, over the duration of culture in the flasks, the NK cells of the NK cell fraction are cultured at a cell density of 100-3000×10⁷ cells per flask. In some embodiments, the fold-expansion of the NK fraction within the expanded, combined CD3-depleted and CD3+ feeder cell fractions is on the range of 2-15× original cell number. In specific embodiments, the NK cell expansion is approximately 90% of the cells at completion of the expansion cycle, and the fold increase is 2-10-fold. In particular embodiments, the fold increase of the NK cells during expansion is 2-6×, 3-5, 4-6 or 4-5 times the number of NK cells in the apheresis unit prior to culturing.

Culturing the NK cells can be effected with or without feeder cells or a feeder cell layer. The present inventors have shown that culturing the CD3-depleted fraction along with its inactivated, corresponding CD3+ fraction enhances the proliferation and functionality of the expanded NK cell population. Thus, according to one embodiment, culturing the population of NK cells is effected with feeder layer or feeder cells.

In certain embodiments, the CD3-depleted NK cells are harvested from the culture 14-16 days following initiation of the CD3-depleted and CD3+ fraction cell culture (step (b)). Harvesting of the cells can be performed manually, by releasing attached cells (e.g. “scraping” culture vessel surfaces) or by a cell harvesting device, which is designed to efficiently wash cells out of their culture vessels and collect the cells automatically. In specific embodiments, the expanded CD3-depleted NK cell fraction is harvested from the culture vessels by a cell harvesting device (e.g. the LOVO Cell Processing device by Fresenius Kabi (Hamburg, Germany)).

In some embodiments, harvesting of expanded CD3-depleted, NK-cell enriched (through expansion) fraction from culture removes most, or nearly all of the cells (including the irradiated CD3+ feeder cells) from the culture vessel. In other embodiments, harvesting can be performed in two or more steps, allowing the unharvested cells to remain in culture until harvested at a later time. In certain embodiments, the expanded CD3-depleted NK cell-containing fraction is harvested in two steps, comprising harvesting a first portion of the expanded CD3-depleted NK-containing cell fraction, and then harvesting a second portion of the expanded CD3-depleted cell fraction. Harvesting the two portions can be performed with an interval of hours, days or more between harvesting of the first and second portion. In certain embodiments, harvesting comprises harvesting a first portion of the expanded CD3-depleted NK cells about 14 days following step (b)(initiation of culturing), and harvesting a second portion of the expanded CD3− depleted cell fraction about 2 days later. In a specific embodiment, the first portion is harvested 14 days following initiation of the ex-vivo culture and the second portion is harvested 16 days following initiation of the ex-vivo culture.

In certain embodiments, the first and second portions are approximately equal, namely, the first (harvested) portion comprises about 50% of the expanded CD3-depleted NK cell fraction and the second (harvested) portion comprises the remainder of the expanded CD3− depleted NK cell fraction.

In specific embodiments, the entire cell contents of the culturing vessel (e.g. flask) is harvested at the same time. In some embodiments, the cells are harvested on day 14, or 15 or 16 following seeding.

In order to prepare the expanded combined CD3-depleted and CD3+ feeder cell population for further use, (e.g. transplantation (infusion), or cryopreservation), the harvested cells need to be washed of culture medium, critical parameters evaluated, and volume adjusted to a concentration suitable for the further processing.

Following harvesting, the expanded combined CD3-depleted and CD3+ feeder cell population can be washed free of culture medium manually or, preferably for clinical applications, using an automated device employing a closed system. For immediate use, washed cells can be reconstituted with an infusion solution (exemplary infusion solutions suitable for use with the method described herein include, but are not limited to CSB buffer (Biolife Solutions, Bothell, WA), 2-8 Cellsius buffer (Protide Pharmaceuticals, Lake Zurich, IL) and 8% w/v HSA and 6.8% w/v Dextran-40), saline or other reconstitution/infusion buffer such as PlasmaLyte (Baxter Healthcare Ltd). In some aspects of the methods of the present disclosure, an infusion solution can be PlasmaLyte. As would be appreciated by the skilled artisan, PlasmaLyte comprises Sodium Chloride at a concentration of 5.26 g/1, Potassium Chloride at a concentration of 0.37 g/1, Magneisum Chloride hexahydrate at a concentration of 0.30 g/1, Sodium Acetate trihydrate at a concentration of 3.68 g/l and Sodium Gluconate at a concentration of 5.02 g/l and as a pH of about 7.4 (in the range of 6.5 to 8.0).

Thus, in some embodiments, there is provided a CD3-depleted cell fraction comprising expanded NK cells, suitable for infusion into a subject, prepared according to the methods described herein.

For cryopreservation, the washed cells can be reconstituted with cryopreservation buffer. In some embodiments, the cells are first washed and reconstituted (e.g. suspended) in cryopreservation buffer solution without DMSO (e.g. CSB buffer, Biolife). The washed cells can then be sampled for evaluation of viability, function, potency, sterility cell count and the like, before reducing temperature, according to procedures described herein in detail.

The present inventors have shown that NK cells expanded with nicotinamide and CD3+ feeder cells, according to the methods described herein, can be efficiently cryopreserved while retaining their phenotype, functionality and viability, suitable for thawing and therapeutic use after cryogenic storage.

Thus, according to some aspects of the invention, there is provided a method for cryopreservation of NK cell fractions comprising:
(a) suspending NK cells of an NK cell fraction in a DMSO-free cryopreservation buffer;
(b) chilling the cells for 10-30 min at 4° C.;
(c) adding DMSO to 10% w/v;
(d) reducing temperature of the cells to −120° C., and
(e) storing the cryopreserved NK cell at <120° C.

In particular embodiments, the NK cell fraction for cryopreservation is an expanded NK cells fraction. In specific embodiments, the NK cell fraction for cryopreservation is the expanded NK cell fraction of the CD3-depleted and inactivated CD3+ feeder cells cultured, harvested and washed according to the methods described herein.

Thus, according to some aspects of the invention, there is provided a method for preparing a cryopreserved NK cell fraction for a subject in need thereof, comprising:
(a) obtaining an apheresis product comprising NK cells and CD3+ cells allogeneic to subject;
(b) separating the apheresis product into a CD3-depleted cell fraction and a CD3+ cell fraction;
(c) inactivating cells of said CD3+ cell fraction by irradiation;
(d) ex vivo culturing said CD3-depleted cell fraction with the inactivated, irradiated CD3+ cell fraction under conditions allowing for cell proliferation, wherein said conditions comprise providing nutrients, serum, IL-15, a CD3 agonist and nicotinamide in an amount between 1.0 mM to 10 mM;
(e) supplementing said combined CD3-depleted and CD3+ cell fractions with fresh nutrients, serum, IL-15 and nicotinamide 6-10 days following step (d) to produce an expanded CD3-depleted cell fraction;
(f) harvesting said combined CD3-depleted and CD3+ cell fractions 14-16 days following step (d); and
(g) washing and concentrating said combined CD3-depleted and CD3+ cell fraction of step (f), and
(h) freezing and cryopreserving the combined CD3-depleted and CD3+ cell fraction of step (g), thereby producing a cryopreserved NK cell fraction for a subject in need thereof. The cryopreserved NK cell fraction prepared according to the described method is suitable for administration in the clinical setting.

Prior to reducing temperature for freezing, a suitable cryoprotectant (for example, Human Serum Albumin, and/or DMSO) is added to the cells to complete the cryopreservation buffer. In some embodiments, the cryopreservative solution is or contains, for example, at least or about 1 to 30% DMSO. In some embodiments, the cells are formulated with a cryopreservative solution that contains 1.0% to 30% DMSO solution, such as a 5% to 20% DMSO solution or a 5% to 10% DMSO solution. In some embodiments, the cryopreservation solution, including the cryopreservative is or contains, for example, CSB buffer (Biolife Solutions, Bothell, WA) containing 10% DMSO, or other suitable cell freezing media.

In some embodiments, the processing can include dilution or concentration of the cells to a desired concentration or number, such as unit dose form compositions including the number of cells for administration in a given dose or fraction thereof. In some embodiments, the processing steps can include a volume-reduction to thereby increase the concentration of cells as desired. In some embodiments, the processing steps can include a volume-addition to thereby decrease the concentration of cells as desired.

In some embodiments, the processing for cryopreservation includes adding a volume of a cryopreservation buffer to the expanded cells or replenishing the volume to a desired final volume in the cryopreservation bag. In some embodiments, the volume of formulation buffer is from or from about 10 mL to 1000 mL, such as at least or about at least or about or 20 mL, 30 mL, 40 mL, 50 mL, 100 mL, 200 mL, 300 mL, 400 mL, 500 mL, 600 mL, 700 mL, 800 mL, 900 mL or 1000 mL. In particular embodiments, the volume of the suspended cells for cryopreservation, including the buffer, is 20 ml.

In some embodiments, the expanded CD3-depleted and CD3+ feeder layer cells are transferred to a container, such as a bag that is operably linked as part of a closed cell-processing system. In some embodiments, the container, such as bag, is connected to a system at an output line or output position. In some embodiments, the bag is a cryopreservation bag, specifically a fluorinated ethylene propylene cryopreservation bag. A variety of suitable cryopreservation bags is commercially available, for example, the CryoMACS® cryopreservation bag (Miltenyi Biotech) or the OriGen cryopreservation bag (Abacus). In specific embodiments, the cryopreservation bag is a CryoMACS cryopreservation bag. In some aspects, the cryopreservation bag can be a 250 ml cryopreservation bag. In some aspects, the cryopreservation bag can be a 50 ml cryopreservation bag.

In some embodiments, the closed system, such as associated with a cell processing system, includes a single-port outlet, or a multi-port output kit containing a multi-way tubing manifold associated at each end of a tubing line with a port to which one or a plurality of containers can be connected for transfer of the expanded cell composition. In some aspects, a desired number or plurality of output containers, e.g., cryopreservation bags, can be sterilely connected to one or more, generally two or more, such as at least 3, 4, 5, 6, 7, 8 or more of the ports of the multi-port output. For example, in some embodiments, one or more containers, e.g., bags can be attached to the ports, or to fewer than all of the ports. Thus, in some embodiments, the system can effect transfer of the cultured and expanded CD3-depleted cells into a plurality of output bags, and, if desired, the subject.

In some embodiments, each of the containers, e.g., bags, individually comprises a unit dose of the cells. Thus in some embodiments, each of the containers comprises the same or approximately or substantially the same number of cells. In some embodiments, the unit dose includes equal to or about 5×10⁸, 2.5×10⁸, 1×10⁸, 5×10⁷, 2×10⁶ or less than about 5×10⁵ of total cells/ml. In some embodiments, the volume of the cells plus the cryopreservation buffer in each bag is 10 mL to 100 mL, such as at least or about at least 20 mL, 30 mL, 40 mL, 50 mL, 60 mL, 70 mL, 80 mL, 90 mL or 100 mL. In some embodiments, each unit dose contains about 2.5×10⁸ total cells in 20 ml volume of cryoprotectant buffer (e.g. CSB buffer).

As used here, the term cryogenically freezing means lowering the temperature of a sample, for example, a sample containing cells, to a temperature from −80° C. to −210° C.

Freezing of the expanded CD3-depleted and irradiated CD3+ feeder cell population is affected in stages: first, reducing the temperature of the cells (in the sealed cryopreservation bags) to −120° C. (minus 120° C.). The reduction in temperature is performed gradually, to prevent shock and damage to the cells. In some embodiments, the reduction to −120° C. is affected in two stages: gradual freezing, for example, at −1° C./min, until −60° C., followed by more rapid freezing, for example, at −5° C./min until the target temperature (−120° C.). The frozen cryopreservation bags comprising the cryopreserved cells are then transferred to a cryopreservation device, such as a liquid nitrogen flask (e.g. the vapor phase of a liquid nitrogen flask) or ultra-low freezer for storage at less than −120° C., and in some embodiments, less than −140° C. or less than or equal to −150° C.

In some embodiments, an intermediate step of “snap freezing” is introduced, during the gradual reduction in temperature (for example, prior to reaching −60° C.), comprising rapid sudden temperature reduction, and then return to the gradual freezing.

As used here, the term “cryogenically storing” or “cryogenic storage” generally refers to storing a sample, for example, a sample containing cells at a temperature from −80° C. to −210° C. and in a condition such that the cells are capable of being thawed after a period of such storage, such that upon or following thawing, at least a portion of or substantial portion of cells in the sample remain viable and/or retain at least a portion of a biological function thereof. In one aspect, the cell sample is capable of being thawed such that at least a certain percentage, such as at or about or more than 20%, 30%, 40%, 50%, 60%, 70%, 80%, 90%, or 100% of the cells in the sample remain viable.

Thus, in some embodiments, there is provided a cryopreserved CD3-depleted cell fraction comprising expanded NK cells, suitable for administration (e.g. by infusion), prepared according to the methods described herein. In some embodiments, the cryopreserved CD3− depleted cell fraction comprising expanded NK cells is provided in a fluorinated, ethylene propylene cryopreservation bag comprising 0.5×10⁸ total cells/ml, 1.0×10⁸ total cells/ml, 1.5×10⁸ total cells/ml, 2.0×10⁸ total cells/ml, 2.5×10⁸ total cells/ml, 3.0×10⁸ total cells/ml, 3.5×10⁸ total cells/ml or 4.0×10⁸ total cells/ml, in a volume of 5, 10, 15, 20, 25, 30, 35, 40, 50, 60, 70, 90, 100 ml or more. In some embodiments, the cryopreservation bag can be of a volume of 10, 20, 30, 40, 50, 60, 70, 80, 100 or in the range of 50-1000, 50-500, 100-500, 50-250 ml. In specific embodiments, the cryopreserved CD-depleted cell fraction comprising expanded NK cells is provided in a cryopreservation bag of 250 or 50 ml. In specific embodiments, the cryopreserved CD3-depleted cell fraction comprising expanded NK cells is provided in a fluorinated, ethylene propylene cryopreservation bag comprising 2.5×10⁸ total cells/ml in a volume of 20 ml.

It will be appreciated that some of the cryopreserved fractions can be thawed and sampled at various times following cryopreservation, in order to monitor the viability, characteristics, function, potency and cell density (number/volume) over the period of storage. Sampling can be done at regular intervals (once every month, two months, six months, or more).

In some aspects, a single cryopreservation bag is thawed only once.

In order to provide accurate characterization of the stored, cryopreserved cell populations, the parameters of individual cryopreserved fractions (potency, contamination, sterility, viability, appearance, phenotype, donor statistics, etc.) may be recorded, for reference in selection of cryopreserved units for thawing later on.

The cells may be cryogenically stored in a manner according to the embodiments described above, such as in the vapor phase of a liquid nitrogen storage tank, and such as for a storage period of from 1 day to 12 years. In some embodiments, the cells are stored, or banked, for a period of time greater than or equal to 12 hours, 24 hours, 36 hours, or 48 hours. In some embodiments, the cells are stored or banked for a period of time greater than or equal to 1 week, 2 weeks, 3 weeks, or 4 weeks. In some embodiments, the cells are placed into long-term storage or long-term “banking”. In some aspects, the cells are stored for a period of time greater than or equal to 1 month, 2 months, 3 months, 4 months, 5 months, 6 months, 7 months, 8 months, 9 months, 10 months, 11 months, 1 year, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 25 years, 30 years, 35 years, 40 years, or more.

In some embodiments, after the storage period, the cells are thawed. In some embodiments, the cells are thawed by raising the temperature of the cells to at or above 0° C., so as to restore at least a portion of a biological function of the cells. In some embodiments, the cells are thawed by raising the temperature of the cells to 37° C., so as to restore at least a portion of a biological function of the cells.

In some embodiments, the cells are thawed rapidly, e.g., as rapidly as possible without overheating the cells or exposing the cells to high temperatures such as above 37° C. In some embodiments, rapid thawing reduces and/or prevents exposure of the cells to high concentrations of cryoprotectant and/or DMSO. In particular embodiments, the rate at which thawing occurs may be affected by properties of the container, e.g., the vial and/or the bag, that the cells are frozen and thawed in.

In some embodiments, the cells are thawed on a heat block, in a dry thawer, or in a water bath. In certain embodiments, the cells are not thawed on a heat block, in a dry thawer, or water bath. In some embodiments, the cells are thawed at room temperature. In particular embodiments, the cells are thawed at a temperature of, of about, or less than 37° C., 35° C., 32° C., 30° C., 29° C., 28° C., 27° C., 26° C., 25° C., 24° C., 23° C., 22° C., 21° C., 20° C., or 15° C., or between 15° C. and 30° C., between 23° C. and 28° C., or between 24° C. and 26° C., each inclusive.

In some embodiments, cryofrozen cells are rapidly thawed. In particular embodiments, the cells are thawed in, in about, or in less than 120 minutes, 90 minutes, 60 minutes, 45 minutes, 30 minutes, 25 minutes, 20 minutes, 15 minutes, or ten minutes. In some embodiments, the cells are thawed for between 10 minutes and 60 minutes, 15 minutes and 45 minutes, or 15 minutes and 25 minutes, each inclusive. In particular embodiments, the cells are thawed in, in about, or in less than 20 minutes.

In some embodiments, the cells are thawed on a heat block, in a dry thawer, or in a water bath. In certain embodiments, the cells are not thawed on a heat block, in a dry thawer, or water bath. In some embodiments, the cells are thawed at room temperature. In specific embodiments, the cells are incubated for 2-15 minutes at room temperature. In particular embodiments, the cryopreservation bags with the cells are incubated at room temperature (e.g. 15-25° C.) for 5 minutes, and then thawed in a 37° C. water bath until thawed—e.g. all solids have been completely dissolved into solution.

In certain embodiments, the thawed cells are rested, e.g., incubated or cultured, prior to administration or prior to any subsequent engineering and/or processing steps. In some embodiments, the cells are rested in low and/or undetectable amounts of cryoprotectant, or in the absence of cryoprotectant, e.g., DMSO. In particular embodiments, the thawed cells are rested after or immediately after washing steps, e.g., to remove cryoprotectant and/or DMSO. In some embodiments, the cells are rested for, for about, or for at least 5 minutes, 10 minutes, 15 minutes, 30 minutes, 1 hour, 2 hours, 3 hours, 4 hours, 5 hours, 6 hours, 8 hours, 12 hours, 18 hours, or 24 hours. In certain embodiments, the cells are rested for, for about, or for at least 2 hours.

Thawed cells can be reconstituted with any suitable infusion, buffer, saline, PlasmaLyte etc. solution. In some embodiments, after thorough thawing, the cells are diluted with infusion solution (e.g. PlasmaLyte® (Baxter) or lactated Ringer's solution), allowing direct use in treatment. In other embodiments, the cells are diluted with a solution comprising Dextran and human serum albumin (HSA).

In particular embodiments, the cells are diluted after thawing with a commercially available electrolyte infusion solution, such as PlasmaLyte (Baxter), lactated Ringer's solution and the like. In some embodiments, the thawed cells are diluted with PlasmaLyte (Baxter) in a ratio of 1:4 cells to diluent. In some embodiments, 20 ml of thawed cells are diluted with 80 ml of PlasmaLyte, to a final volume of 100 ml.

In some embodiments, the reconstitution is performed in a closed system. In some embodiments, the infusion solution is screened for suitability for use with the methods and compositions of the present invention. Exemplary criteria for selection of suitable infusion solution include safety tests indicating no bacterial, yeast or mold growth, endotoxin content of less than 0.5 Eu/ml and a clear, foreign particle-free appearance. In some aspects, NK cell fractions and compositions comprising NK fractions of the present disclosure comprise no more than about 0.5 Endotoxin Units (EU)/ml.

As used herein, the term “propagation” or “proliferation” refers to growth, for example, cell growth, and multiplication of cell numbers. Propagation and proliferation, as used herein relate to increased numbers of NK cells accruing during the incubation period. Propagation in vitro or in vivo of cells displaying the phenotype of NK cells is a known phenomenon following their stimulation, for example with IL-2, Epstein-Barr virus-transformed lymphoblastoid lines, RAJI and K562 cells and others.

Assays for cell proliferation are well known in the art, including, but not limited to clonogenic assays, in which cells are seeded and grown in low densities, and colonies counted, mechanical assays [flow cytometry (e.g., FACS™), propidium iodide], which mechanically measure the number of cells, metabolic assays (such as incorporation of tetrazolium salts e.g., XTT, MTT, etc.), which measure numbers of viable cells, direct proliferation assays (such as bromodeoxyuridine, thymidine incorporation, and the like), which measure DNA synthesis of growing populations. In one embodiment, cell proliferation of populations of NK cells cultured with an effective concentrations of nicotinamide and/or other nicotinamide moiety according to the present invention is measured at a predetermined time after seeding the CD3-depleted cells in culture (for example, about 10 hours, 12 hours, about 1, 2, 3, 4, 5, 6, 7 days, about 1, 2, 3, 4, 5 weeks, 2 months or more) is determined by FACS analysis, using anti-CD56 and anti-CD3 markers to identify and quantitate the CD56+CD3− NK cell fraction of the population. Proliferation of NK cells can be expressed as the fold increase, (e.g., expansion or fold expansion) of NK cells, as compared to the original NK cell fraction before culture. In some embodiments, populations of NK cells exposed to effective concentrations of nicotinamide according to the present invention have a fold increase of the NK cell population of at least 2×, at least 10×, at least 20×, at least 40×, at least 50×, at least 75×, at least 100×, at least 150×, at least 250× and at least 500× or more, after about 5, about 7, about 12, about 14, about 16, about 18, about 21, about 25, about 30 or more days culture. In another embodiment, the fold expansion of populations of NK cells, as determined by FACS™, exposed to effective concentrations of nicotinamide is at least about 1.2×, about 1.3×, about 1.5×, about 1.75×, about 2×, about 2.25×, about 2.5×, about 2.75×, about 3.0, about 3.5×, about 4×, about 4.5×, about 5×, about 6×, about 7×, about 8×, about 9×, about 10×, more than that of NK cells cultured in identical conditions with less than 0.1 mM nicotinamide and/or other nicotinamide moiety.

As used herein, the term “function” or “NK cell function” refers to any biological function ascribed to NK cells. A non-limiting list of NK cell functions includes, for example, cytotoxicity, induction of apoptosis, cell motility, directed migration, cytokine and other cell signal response, cytokine/chemokine production and secretion, expression of activating and inhibitory cell surface molecules in-vitro, cell homing and engraftment (in-vivo retention) in a transplanted host, and alteration of disease or disease processes in vivo. In some embodiments, NK cell functions enhanced by exposure to nicotinamide and/or other nicotinamide moiety include at least one of elevated expression of CD62L surface marker, CD107a, elevated migration response, and greater cytotoxic activity of the NK cells, as well as elevated homing and in-vivo retention of infused NK cells.

Assays for adhesion and migration molecules such as CD62L, CXCR-4, CD49e and the like, important for homing/engraftment and retention of cells in transplantation, are well known in the art. CD62L expression in a cell can be assayed, for example, by flow cytometry, immunodetection, quantitative cDNA amplification, hybridization and the like. In one embodiment, CD62L expression is detected in different populations of NK cells by exposure of the cells to a fluorescent-tagged specific anti-human CD62L monoclonal antibody [e.g., CD62L PE, Cat. No. 304806 from BioLegend (San Diego, CA, USA)], and sorting of the cells by fluorescent activated cell sorting (FACS).

Assays for cells migration are well known in the art. Migration of cells can be assayed, for example, by transmigration assays or gap closure assays. In transmigration assays, such as the two-chamber technique, cells are separated from a stimulus by a barrier (e.g., filter), and migration of the cells is detected by counting loss of cells from the origin, accumulation of cells across the barrier, or both, at specific intervals. In the gap closure assay, cells are placed on the periphery of a visible gap (scored agar plate, around a circle, etc.) and incubated with a stimulus. Closure of the space between the cells applied by cell motility, in response to a stimulus, is visualized using cytometry, immunodetection, microscopy/morphometrics, etc. In one embodiment, migration potential of different populations of NK cells is determined by the “Transwell”™ transmigration assay, in response to SDF (250 ng/ml).

Assays for homing and in-vivo retention of transfused or transplanted cells are well known in the art. As used herein, the term “homing” refers to the ability of a transfused or transplanted cell to reach, and survive, in a host target organ. For example, NK cells target organs can be the lymphoid tissue, hepatocytes target organs can be liver parenchyma, alveolar cells target organs can be lung parenchyma, etc. As used herein, the term “in-vivo retention” (also known as “engraftment”) refers to the ability of the transfused or transplanted cells to proliferate and remain viable in the target organs. Animal models for assaying homing and in-vivo retention of transplanted NK cells include, but are not limited to immunodeficient small mammals (such as SCID and IL2Rγ^{null} mice and the like). The SCID-Hu mouse model employs C.B-17 scid/scid (SCID) mice transplanted with human fetal thymus and liver tissue or fetal BM tissue and provides an appropriate model for the evaluation of transplanted human NK cells retention and therapeutic potential. Homing and in-vivo retention of transplanted cells can be assessed in human host subjects as well. In one embodiment, homing and in-vivo retention is assayed in irradiated NOD/SCID mice, transfused with, for example, about 15×10⁴, about 15×10⁵, about 15×10⁶, about 15×10⁷ or more human NK cells cultured with an effective concentrations of nicotinamide according to the present invention, and sacrificed at a predetermined time post transfusion (for example, about 5 hours, 10 hours, 12 hours, 1, 2, 3, 4, 5, 6, 7 days, 1, 2, 3, 4, 5 weeks, 2, 3, 4 months or more post transfusion). Upon sacrifice of the mice, samples of spleen, bone marrow, peripheral blood, and other organs are evaluated by FACS for the presence of human NK cells (CD56+CD45+) using human specific Abs. Percent in vivo retention is expressed as the percent of cells of the organ displaying the donor phenotype (e.g., CD45 for human cells). In specific embodiments, in-vivo retention of NK cells expanded, cryopreserved and thawed according to the methods described herein have increased retention in the spleen, as compared to NK cells cultured without nicotinamide and a CD3+ feeder layer.

Assays for cytotoxicity (“cell killing”) are well known in the art. Examples of suitable target cells for use in redirected killing assays are cancer cell line, primary cancer cells solid tumor cells, leukemic cells, or virally infected cells. Particularly, K562, BL-2, colo250, RAJI (lymphoblast-like Burkitt's lymphoma cells) and primary leukemic cells can be used, but any of a number of other cell types can be used and are well known in the art (see, e.g., Sivori et al. (1997) J. Exp. Med. 186: 1129-1136; Vitale et al. (1998) J. Exp. Med. 187: 2065-2072; Pessino et al. (1998) J. Exp. Med. 188: 953-960; Neri et al. (2001) Clin. Diag. Lab. Immun. 8:1131-1135). Cell killing is assessed by cell viability assays (e.g., dye exclusion, chromium release, CFSE), metabolic assays (e.g., tetrazolium salts), and direct observation.

Once the expanded CD3-depleted cell fraction has been thawed and diluted, the expanded fraction can be evaluated for suitability for use in transplantation. Typical criteria for selection of suitable transplantable NK cell preparations include the percentage of CD56+/CD3− cells, cell viability, amount of the CD3+ cell fraction, presence of endotoxin, microbial contamination and the like. It will be noted that the CD56+, CD3+ and CD56+/CD3− cell content of the expanded NK cell fraction is critical to the successful homing and retention of the infused NK cells, and is thus a central criterion for proceeding to infusion. Thus, in particular embodiments, the thawed and diluted CD3-depleted cell fraction is characterized by about 60% to about 90% CD56+/CD3− cells, about 68% to about 85% CD56+/CD3− cells, about 72% to about 82% CD56+/CD3− cells and about 76-79% CD56+/CD3− cells. In one embodiment, the thawed and diluted CD3− depleted cell fraction of the invention is characterized by at least 60%, at least 64%, at least 70%, at least 74%, at least 80% or at least 85% CD56+/CD3− cells. In a further embodiment, the thawed and diluted CD3-depleted cell fraction generated by the method of the invention is characterized by at least 70% CD56+/CD3− cells. Identification of NK cells phenotype according to CD56 and CD3 cell markers is described in detail hereinabove.

The presence of allogeneic T (CD3+) cells in cell fractions intended for transplantation is problematic since they strongly increase the risk of GVHD. Thus, an important parameter for suitability of expanded NK cell fractions for clinical use is the amount or fraction of CD3+, and, in particular, CD3+/CD56− cells. Thus, in particular embodiments, the thawed and diluted CD3− depleted cell fraction of the invention is characterized by between 1.0×10⁵ and 1.0×10⁶ CD3+/CD56− cells per Kg mass of the patient. In further embodiments, the thawed and diluted CD3-depleted cell fraction generated by the methods of the invention is characterized by fewer than 7.0×10⁵ CD3+/CD56− cells per Kg mass of the patient, fewer than 6.5×10⁵ CD3+/CD56− cells per Kg mass of the patient, fewer than 6.0×10⁵ CD3+/CD56− cells per Kg mass of the patient, fewer than 5.5×10⁵ CD3+/CD56− cells per Kg mass of the patient, fewer than 5.0×10⁵ CD3+ cells per Kg mass of the patient, fewer than 4.5×10⁵ CD3+/CD56− cells per Kg mass of the patient, fewer than 4.0×10⁵ CD3+/CD56− cells per Kg mass of the patient, fewer than 3.5×10⁵ CD3+ cells per Kg mass of the patient or fewer than 3.0×10⁵ CD3+CD56− cells per Kg mass of the patient. In one embodiment, the thawed and diluted CD3-depleted cell fraction generated by the methods of the invention is characterized by fewer than 1×10⁶ CD3+CD56− cells per Kg mass of the patient. In other embodiments, the thawed and diluted CD3-depleted cell fraction generated by the methods of the invention is characterized by fewer than 0.5×10⁶ CD3+CD56− cells per Kg mass of the patient. It will be noted that calculation of the CD3+CD56− fraction, portion or content of the thawed and diluted CD3-depleted cell fraction of the invention, expressed per Kg mass of the patient, relates to the total amount of CD3+CD56− cells transplanted (e.g. infused) into the patient (i.e. subject). The fraction, portion or amount of CD3+CD56− cells in the thawed and diluted CD3-depleted cell fraction of the invention can also be expressed as a ratio of CD56+/CD3− to CD3+CD56− cells, or as a volume fraction (e.g. CD3+/CD56− cells/mL) or weight fraction (CD3+/CD56− cells/100 g) of the thawed and diluted CD3-depleted cell fraction of the invention. Identification of CD3+ cell markers is described in detail hereinabove.

Sterility and safety of the expanded, CD3-depleted NK cell fractions for transplantation is assured by monitoring, inter alia, the endotoxin content and presence of bacterial, fungal, viral and mycoplasma contamination. In some embodiments, the expanded NK cell fraction selected for transplantation has an endotoxin content of no more than 5 Eu/ml after thawing and dilution. In some embodiments, the thawed and diluted CD3-depleted cell fraction for transplantation is characterized as being free of microorganisms (for example, Gram-positive microorganisms) following thawing and dilution. In particular embodiments, the thawed and diluted CD3− depleted cell fraction of the invention is sterile, and free of mycoplasma after thawing and dilution.

In some embodiments, the expanded NK cell fraction is characterized by about 50% to about 85% viability. In some embodiments, expanded NK cell fractions having about 55%, about 60%, about 63%, about 65%, about 68%, about 70%, about 75%, about 78%, about 80%, about 82%, about 83%, about 84% to about 85% viability or greater are selected. In a further embodiment, the NK cell fraction (e.g. CD3-depleted fraction) selected for ex-vivo expansion has at least 70% viable cells. In a further embodiment, the expanded NK cell fraction (e.g. combined CD3-depleted and CD3+ cells) suitable for clinical applications is characterized by at least 70% viable cells following washing and concentration. In a further embodiment, the expanded NK cell fraction suitable for transplantation has at least 85% viable cells.

As used herein, the term “viability” refers to the distinction between living and non-living cells. Cell viability may be judged by morphological changes or by changes in membrane permeability and/or physiological state inferred from the exclusion of certain dyes or the uptake and retention of others. Cell viability assessment is well known in the art, including, but not limited to assays (e.g., dye exclusion, chromium release), metabolic assays (e.g., tetrazolium salts), and direct observation. (Coder, D., Current Protocols in Cytometry, 1997, John Wiley and Sons, Inc., Unit 9.2, 9.2.1-9.2.14).

In some embodiments, the parameters of CD56+/CD3− cell fraction, CD3+ cells fraction, viability, endotoxin and microorganism content, as well as phenotype, NK cell function, potency and appearance are monitored in samples drawn prior to seeding for cell culture, during cell culture, after harvesting, following wash and concentration of the expanded CD3-depleted cell fractions, following cryopreservation, following storage, and in the thawed and diluted CD3− depleted cell fraction of the invention. In some embodiments, the samples are drawn from any of the apheresis unit before processing (e.g. 100×10⁶ cells), post-column (CD3 depletion) pre culture sample (e.g. 10×10⁶ cells), post-expansion-pre-wash (e.g. 10 ml sample), final expanded, washed and concentrated CD3-depleted cell product (10×10⁶ cells), following cryopreservation, during storage, following thawing and dilution, and/or on the day of first infusion (Day 0) and, if needed, on the day of any further infusions, or any combination thereof.

The present inventors have shown that culturing the CD3-depleted cells with nicotinamide and irradiated CD3+ feeder cells results in robust expansion of the NK cell fraction, as well as enhancement of the NK cells functionality and homing/engraftment potential (see Examples 1-4). Thus, in some embodiments, the expanded CD3-depleted cells have increased expression of CD62L, as compared to CD3-depleted cells expanded under identical conditions having 5 mM nicotinamide and without said CD3+ cell fraction. In some embodiments, the thawed and diluted CD3-depleted cell fraction of the invention have increased expression of CD62L, as compared to CD3-depleted cells expanded under identical conditions having 5 mM nicotinamide and without said CD3+ cell fraction.

In other embodiments, the expanded CD3-depleted cells have increased in-vivo retention in the spleen and bone marrow following infusion into irradiated mice compared to CD3− depleted cells expanded under identical conditions having 5 mM nicotinamide and without said CD3+ cell fraction. In further embodiments, the thawed, cryopreserved NK cell fraction of claim 25, wherein said expanded CD3-depleted cells have increased in-vivo retention in the spleen and bone marrow following infusion into irradiated mice compared to CD3-depleted cells expanded under identical conditions having 5 mM nicotinamide and without said CD3+ cell fraction.

Thus, according to specific embodiments, the thawed and diluted CD3-depleted cell fraction of the invention is characterized by the following parameters:
(a) at least 70% CD56+/CD3− cells;
(b) at least 70% viability;
(c) fewer than 1×10⁶ CD3+CD56− cells/Kg mass of patient, upon infusion;
(d) no more than 5 EU endotoxin/Kg mass of patient, upon infusion;
(e) no mycoplasma, and
(f) sterile.

Thus, according to specific embodiments, the thawed and diluted CD3-depleted cell fraction of the invention is characterized by the following parameters:
(a) at least 70% CD56+/CD3− cells;
(b) at least 70% viability;
(c) less than 0.5% of cells are CD3+/CD56−;
(d) no more than 0.5 Endotoxin Units (EU)/mL;
(e) no mycoplasma, and
(f) sterile.

Expanded CD3-depleted cell fractions meeting the abovementioned criteria can be used for infusion into subjects (e.g. patients) in need thereof. Any of the methods for ex-vivo expansion (culturing), selection and preparation of CD3-depleted cell fractions for administration described hereinabove, and each of their embodiments taken alone or in various combinations may be used for affecting the methods for administering (e.g. transplanting, infusing) expanded CD3-depleted cell fractions as is described in this section and the sections that follow.

Thus, in some embodiments, there is provided a thawed, cryopreserved NK cell fraction prepared according to any of the methods for preparing a thawed and diluted CD3-depleted cell fraction of the invention described herein. In specific embodiments, the thawed, cryopreserved CD3-depleted NK cell fraction is characterized by the following parameters:
(a) at least 70% CD56+/CD3− cells;
(b) at least 70% viability;
(c) fewer than 1×10⁶ CD3+/CD56− cells/Kg mass of patient, upon infusion;
(d) no more than 5 EU endotoxin/Kg mass of patient, upon infusion;
(e) no mycoplasma, and
(f) sterile.

Thus, in some embodiments, there is provided a thawed, cryopreserved NK cell fraction prepared according to any of the methods for preparing a thawed and diluted CD3-depleted cell fraction of the invention described herein. In specific embodiments, the thawed, cryopreserved CD3-depleted NK cell fraction is characterized by the following parameters:
(a) at least 70% CD56+/CD3− cells;
(b) at least 70% viability;
(c) less than 0.5% of cells are CD3+/CD56−;
(d) no more than 0.5 Endotoxin Units (EU)/mL;
(e) no mycoplasma, and
(f) sterile.

In some embodiments, following thawing and dilution, the NK cell fraction is provided in a container (e.g. for transfer to the site of transplantation (infusion)). In some embodiments, the container is a culture bag. Culture bags constructed of inert materials, having high gas permeability and low water loss, flexibility and high optical transmission are desirable. In specific embodiments, the thawed cryopreserved and diluted CD3-depleted cell fraction of the invention transplantable expanded NK cell fraction is provided in a fluorinated ethylene propylene (FEP) culture bag.

In other embodiments, there is provided a transplantable human NK cell fraction characterized by the following parameters:
(a) at least 70% CD56+/CD3− cells;
(b) at least 70% viability;
(c) fewer than 5.0×10⁵ CD3+/CD56− cells/Kg mass of patient, upon infusion;
(d) no more than 5 EU endotoxin/Kg mass of patient, upon infusion; and
(e) no mycoplasma, and
(f) sterile.

In some embodiments, following thawing and dilution, the NK cell fraction is provided in a container (e.g. for transfer to the site of transplantation (infusion)). In some embodiments, the container is a culture bag. Culture bags constructed of inert materials, having high gas permeability and low water loss, flexibility and high optical transmission are desirable. In specific embodiments, the thawed cryopreserved and diluted CD3-depleted cell fraction of the invention transplantable expanded NK cell fraction is provided in a fluorinated ethylene propylene (FEP) culture bag.

In other embodiments, there is provided a transplantable human NK cell fraction characterized by the following parameters:
(a) at least 70% CD56+/CD3− cells;
(b) at least 70% viability;
(c) less than 0.5% of cells are CD3+/CD56−;
(d) no more than 0.5 Endotoxin Units (EU)/mL;
(e) no mycoplasma, and
(f) sterile.

Expanded CD3-depleted cell fractions of the invention can be used for infusion into subjects in need thereof. In some embodiments, the expanded CD3-depleted cell fractions are thawed and diluted CD3-depleted cell fraction of the invention.

As used herein, the term “transplantation”, in the context of cell therapy, adoptive transfer, cellular immunotherapy or the like refers to administration of cells having an expected therapeutic effect to a subject, preferably to a subject in need thereof, for example, as treatment of a patient for a disease or condition. Since such cell therapy comprises introduction of the therapeutic cell fraction into the subject's body via a vascular connection, as used herein, “transplantation” and “administration” of NK cells is equivalent to “infusion”. Typically, therapeutic cell fractions are infused into the subject intravenously, for example, via a central venous catheter (e.g. Hickman catheter). Rate of infusion of the therapeutic cell fraction into the subject can be controlled by a pump, or unassisted, fed by gravity and adjusted by the height differential between the cell faction and the entrance catheter. In some embodiments, the expanded NK cell fraction is transplanted (infused, administered) intravenously, by gravity feed, without a pump or pumps and/or without filters. In some aspects, the expanded NK cell fraction is transplanted (infused/administered) using a filter. In some aspects, the filter can have a pore size of about 170 to about 260 microns.

In some embodiments, the subject in need of transplantation is suffering from a hematological disease. In some embodiments, the subject is suffering from a hematological malignancy. In specific embodiments, hematologic malignancy indicated for treatment with the CD3-depleted, expanded NK cell fraction, thawed and diluted CD3-depleted cell fraction of the invention or methods described herein is non-Hodgkin's lymphoma (NHL). In particular, the hematological malignancy indicated for treatment with the cell fractions or methods of the invention is CD20+-expressing NHL, including but not limited to Follicular leukemia (FL) and high grade B cell lymphoma (HGBCL). In some aspects, the NHL can be diffuse large B cell lymphoma (DLBCL). In some aspects, the NHL can be mantle cell lymphoma (MCL). In some aspects, the HGBCL can be HGBCL, not otherwise specified (HGBCL, NOS). In some aspects, the NHL can be primary mediastinal large B-cell lymphoma (PMBCL).

Thus, in some embodiments, there is provided a method of treating a hematological disease in a subject in need thereof, the method comprising:
(a) administering an anti-cancer monoclonal antibody to the subject;
(b) administering at least one immunosuppressive agent to the subject;
(c) transplanting an allogeneic thawed, cryopreserved expanded NK cell fraction into said subject in need thereof, wherein said allogeneic thawed cryopreserved expanded NK cell fraction has been expanded by ex-vivo culturing with CD3+ cells, nutrients, serum, IL-15, a CD3 agonist and nicotinamide in an amount between 1.0 mM to 10 mM; and
(d) administering IL-2 to said subject,
thereby treating the hematological disease in the subject.

As used herein, a “subject” or “patient” can be any mammal, e.g., a human, a primate, mouse, rat, dog, cat, cow, horse, pig, sheep, goat, camel. In a specific embodiment, the subject is a human. In further embodiments, the subject is human and the CD3-depleted cell fraction is a human CD3-depleted cell fraction.

As used herein, a “subject in need thereof” is a subject having the need for transplantation, transfusion, infusion or implantation of the thawed and diluted CD3-depleted cell fraction of the invention to treat or ameliorate a disease, disorder or condition. In one embodiment, the subject has (been diagnosed with) or suffering from a hematological disease. In some embodiments, the hematological disease is a cell proliferative disorder. In other embodiments, the hematological disease is a hematological malignancy.

As used herein, the term “risk of” or “probability of” refers to the likelihood of an occurrence. In some embodiments, the risk or probability of an occurrence (e.g. tumor shrinkage, response, progression-free survival, and the like) in an individual refers to a risk calculated from comparative data between groups receiving treatment compared to groups not receiving the same treatment. In some embodiments, an increased or decreased risk or probability reflects the difference between treatment and control groups with respect to the outcome under consideration. In some embodiments, an increase or decrease in the risk or probability of a particular occurrence or condition is only relative, and not expressed in numerical values.

As used herein, the term “cell proliferative disorder” refers to conditions in which unregulated or abnormal growth, or both, of cells can lead to the development of an unwanted condition or disease, which may or may not be cancerous. Exemplary cell proliferative disorders of the invention encompass a variety of conditions wherein cell division is deregulated. The term “rapidly dividing cell” as used herein is defined as any cell that divides at a rate that exceeds or is greater than what is expected or observed among neighboring or juxtaposed cells within the same tissue. A cell proliferative disorder includes a precancer or a precancerous condition. A cell proliferative disorder includes cancer. In specific embodiments, the methods and cell compositions provided herein are used to treat or alleviate a symptom of cancer. The term “cancer” includes solid tumors, as well as, hematologic tumors and/or malignancies. In specific embodiments, the hematological malignancy is non-Hodgkin's lymphoma (NHL) or multiple myeloma (MM).

According to yet another aspect of one embodiment of the present invention there is provided a method of inhibiting tumor growth in a subject in need thereof. The method according to this aspect of the present invention is effected by administering a therapeutically effective amount of a population of NK cells of the invention to said subject.

“Treating” or “treatment” includes but is not limited to the administration of an enriched, activated or cultured NK cell composition or population of the present invention to prevent or delay the onset of the symptoms, complications, or biochemical indicia of a disease, alleviating the symptoms or arresting or inhibiting further development of the disease, condition, or disorder (e.g., cancer, metastatic cancer, or metastatic solid tumors). Treatment can be prophylactic, i.e., adjuvant (to prevent or delay the onset of the disease, or to prevent the manifestation of clinical or subclinical symptoms thereof) or therapeutic suppression or alleviation of symptoms after the manifestation of the disease.

In one embodiment, the NK cell population is administered in an amount effective to reduce or eliminate a cancer, such as a solid tumor or a malignancy, or prevent its occurrence or recurrence. “An amount effective to reduce or eliminate the solid tumor or to prevent its occurrence or recurrence” or “an amount effective to reduce or eliminate the hyperproliferative disorder or to prevent its occurrence or recurrence” refers to an amount of a therapeutic composition that improves a patient outcome or survival following treatment for the tumor disease state or hyperproliferative disorder as measured by patient test data, survival data, elevation or suppression of tumor marker levels, reduced susceptibility based upon genetic profile or exposure to environmental factors. “Inhibiting tumor growth” refers to reducing the size or viability or number of cells of a tumor. “Cancer”, “malignancy”, “solid tumor” or “hyperproliferative disorder” are used as synonymous terms and refer to any of a number of diseases that are characterized by uncontrolled, abnormal proliferation of cells, the ability of affected cells to spread locally or through the bloodstream and lymphatic system to other parts of the body (i.e., metastasize) as well as any of a number of characteristic structural and/or molecular features. A “cancerous” or “malignant cell” or “solid tumor cell” is understood as a cell having specific structural properties, lacking differentiation and being capable of invasion and metastasis. “Cancer” refers to all types of cancer or neoplasm or malignant tumors found in mammals, including carcinomas and sarcomas. Examples are cancers of the breast, lung, non-small cell lung, stomach, brain, head and neck, medulloblastoma, bone, liver, colon, genitourinary, bladder, urinary, kidney, testes, uterus, ovary, cervix, prostate, melanoma, mesothelioma, sarcoma, (see DeVita, et al., (eds.), 2001, Cancer Principles and Practice of Oncology, 6th. Ed., Lippincott Williams & Wilkins, Philadelphia, Pa.; this reference is herein incorporated by reference in its entirety for all purposes).

“Cancer-associated” refers to the relationship of a nucleic acid and its expression, or lack thereof, or a protein and its level or activity, or lack thereof, to the onset of malignancy in a subject cell. For example, cancer can be associated with expression of a particular gene that is not expressed, or is expressed at a lower level, in a normal healthy cell. Conversely, a cancer-associated gene can be one that is not expressed in a malignant cell (or in a cell undergoing transformation), or is expressed at a lower level in the malignant cell than it is expressed in a normal healthy cell.

“Hyperproliferative disease” refers to any disease or disorder in which the cells proliferate more rapidly than normal tissue growth. Thus, a hyperproliferating cell is a cell that is proliferating more rapidly than normal cells.

“Advanced cancer” means cancer that is no longer localized to the primary tumor site, or a cancer that is Stage III or IV according to the American Joint Committee on Cancer (AJCC).

“Well tolerated” refers to the absence of adverse changes in health status that occur as a result of the treatment and would affect treatment decisions.

“Metastatic” refers to tumor cells, e.g., human solid tumor or genitourinary malignancy, that are able to establish secondary tumor lesions in the lungs, liver, bone or brain of immune deficient mice upon injection into the mammary fat pad and/or the circulation of the immune deficient mouse.

A “solid tumor” includes, but is not limited to, sarcoma, melanoma, carcinoma, or other solid tumor cancer. “Sarcoma” refers to a tumor which is made up of a substance like the embryonic connective tissue and is generally composed of closely packed cells embedded in a fibrillar or homogeneous substance. Sarcomas include, but are not limited to, chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

“Melanoma” refers to a tumor arising from the melanocytic system of the skin and other organs. Melanomas include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, and superficial spreading melanoma.

“Carcinoma” refers to a malignant new growth made up of epithelial cells tending to infiltrate the surrounding tissues and give rise to metastases. Exemplary carcinomas include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidernoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma viflosum.

“Leukemia” refers to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease—acute or chronic; (2) the type of cell involved; myeloid (myelogenous), lymphoid (lymphogenous), or monocytic; and (3) the increase or non-increase in the number of abnormal cells in the blood—leukemic or aleukemic (subleukemic). Leukemia includes, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia. In specific embodiments, the hematologic malignancy is Non-Hodgkin's Lymphoma (NHL).

Additional cancers include, for example, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, cervical cancer, endometrial cancer, adrenal cortical cancer, and prostate cancer.

In another particular embodiment of this aspect of the present invention the method is affected concomitantly with, following or prior to hematopoietic, hematopoietic progenitor or hematopoietic stem cell transplantation into said subject. In yet further embodiments, the subject is being concomitantly treated with a sensitizing or potentiating agent (e.g., proteasome inhibitor, IL-2, IL-15, etc.) further enhancing the in-vivo function of the transfused NK cells (for details see, for example, US Patent Application 20090104170 to Childs et al).

Decreased numbers and functionality of NK cells in autoimmune patients has been observed, indicating the possibility of NK cell therapy in a variety of autoimmune diseases and conditions (see Schleinitz, et al., Immunology 2010; 131:451-58, and French and Yokohama, Arthrit Res Ther 2004; 6:8-14). Thus, in still another embodiment of the present invention there is provided a method of treating an autoimmune disease or condition in a subject in need thereof. The method according to this aspect of the present invention is effected by administering a therapeutic amount of a population of NK cells of the invention to said subject.

Autoimmune diseases which can be treated by the method of the invention include, but are not limited to cardiovascular diseases, rheumatoid diseases, glandular diseases, gastrointestinal diseases, cutaneous diseases, hepatic diseases, neurological diseases, muscular diseases, nephric diseases, diseases related to reproduction, connective tissue diseases and systemic diseases.

Examples of autoimmune cardiovascular diseases include, but are not limited to atherosclerosis, myocardial infarction, thrombosis, Wegener's granulomatosis, Takayasu's arteritis, Kawasaki syndrome, anti-factor VIII autoimmune disease, necrotizing small vessel vasculitis, microscopic polyangiitis, Churg and Strauss syndrome, pauci-immune focal necrotizing and crescentic glomerulonephritis, antiphospholipid syndrome, antibody-induced heart failure, thrombocytopenic purpura, autoimmune hemolytic anemia, cardiac autoimmunity in Chagas' disease and anti-helper T lymphocyte autoimmunity.

Examples of autoimmune rheumatoid diseases include but are not limited to rheumatoid arthritis and ankylosing spondylitis.

Examples of autoimmune glandular diseases include, but are not limited to, pancreatic disease, Type I diabetes, thyroid disease, Graves' disease, thyroiditis, spontaneous autoimmune thyroiditis, Hashimoto's thyroiditis, idiopathic myxedema, ovarian autoimmunity, autoimmune anti-sperm infertility, autoimmune prostatitis and Type I autoimmune polyglandular syndrome. diseases include but are not limited to autoimmune diseases of the pancreas, Type 1 diabetes, autoimmune thyroid diseases, Graves' disease, spontaneous autoimmune thyroiditis, Hashimoto's thyroiditis, idiopathic myxedema, ovarian autoimmunity, autoimmune anti-sperm infertility, autoimmune prostatitis and Type I autoimmune polyglandular syndrome.

Examples of autoimmune gastrointestinal diseases include, but are not limited to, chronic inflammatory intestinal diseases, celiac disease, colitis, ileitis and Crohn's disease.

Examples of autoimmune cutaneous diseases include, but are not limited to, autoimmune bullous skin diseases, such as, but are not limited to, pemphigus vulgaris, bullous pemphigoid and pemphigus foliaceus.

Examples of autoimmune hepatic diseases include, but are not limited to, hepatitis, autoimmune chronic active hepatitis, primary biliary cirrhosis and autoimmune hepatitis.

Examples of autoimmune neurological diseases include, but are not limited to, multiple sclerosis, Alzheimer's disease, myasthenia gravis, neuropathies, motor neuropathies; Guillain-Barre syndrome and autoimmune neuropathies, myasthenia, Lambert-Eaton myasthenic syndrome; paraneoplastic neurological diseases, cerebellar atrophy, paraneoplastic cerebellar atrophy and stiff-man syndrome; non-paraneoplastic stiff man syndrome, progressive cerebellar atrophies, encephalitis, Rasmussen's encephalitis, amyotrophic lateral sclerosis, Sydeham chorea, Gilles de la Tourette syndrome and autoimmune polyendocrinopathies; dysimmune neuropathies; acquired neuromyotonia, arthrogryposis multiplex congenita, neuritis, optic neuritis and neurodegenerative diseases.

Examples of autoimmune muscular diseases include, but are not limited to, myositis, autoimmune myositis and primary Sjogren's syndrome and smooth muscle autoimmune disease.

Examples of autoimmune nephric diseases include, but are not limited to, nephritis and autoimmune interstitial nephritis.

Examples of autoimmune diseases related to reproduction include, but are not limited to, repeated fetal loss.

Examples of autoimmune connective tissue diseases include, but are not limited to, ear diseases, autoimmune ear diseases and autoimmune diseases of the inner ear.

Examples of autoimmune systemic diseases include, but are not limited to, systemic lupus erythematosus and systemic sclerosis.

In yet another embodiment of the present invention there is provided a method of inhibiting a viral infection in a subject in need thereof. The method according to this aspect of the present invention is effected by (a) ex-vivo culturing a population of NK cells with NK cell growth factors, CD3+ feeder cells and an effective concentration of nicotinamide, as described herein, wherein said effective concentration of nicotinamide enhances proliferation of said NK cells, as compared to said population of cells cultured with growth factors without said concentration of nicotinamide; and (b) administering a therapeutic amount of said cultured NK cells to said subject. Viral infections suitable for treatment with NK cells or NK cell compositions of the invention include, but are not limited to HIV, lymphatic choriomenengitis virus (LCMV), cytomegalovirus (CMV), vaccinia virus, influenza and para-influenza virus, hepatitis (including hepatitis A, hepatitis B, hepatitis C, non-A- non-B, etc.), herpes simplex virus, herpes zoster virus, Theiler's virus and HSV-1. Other infectious diseases suitable for treatment with NK cells or NK cell preparations of the present invention include but are not limited to parasitic infections such as Plasmodium, Leishmania and Toxoplasma infections, and bacterial infections such as mycobacteria and Listeria (for a review of NK cells in treatment of viral, bacterial and protozoan diseases see Zucchini et al., Exp Rev Anti-Infect Ther 2008; 6:867-85, which reference is incorporated by reference herewith).

In some embodiments, the methods and compositions and kits of the present invention can be used for treatment of subjects of all age groups. In specific embodiments, the subject or patient is greater than 18 and less than 70 years of age.

In some embodiments, the subject in need thereof can have multiple myeloma. In further embodiments, the multiple myeloma is (MM) characterized by at least one of the following criteria: (a) relapsed disease between 2-18 months following first autologous stem cell transplantation, (b) relapsed disease at least 4 months following allogeneic stem cell transplantation with no evidence of active graft versus host disease (GVHD), (c) relapsed/refractory disease following at least two lines of therapy including proteasome inhibitor and an immunomodulatory drug (IMiD), (d) Serum IgG, IgA, IgM or IgD Myeloma protein (M-protein) greater than or equal to 0.5 g/dL and (e) Urine M-protein greater than or equal to 200 mg/24 collection. In some embodiments, the multiple myeloma is also characterized by serum IgE Myeloma protein (M-protein) greater than or equal to 0.5 g/dL, and has undergone plasmapheresis no fewer than 4 weeks prior to the start of cell therapy treatment with the thawed and diluted CD3-depleted cell fraction of the invention. In some embodiments, the subject in need thereof has multiple myeloma characterized by more than one of the criteria described herein.

The subject in need thereof can have Non-Hodgkin's lymphoma (NHL). In some embodiments, the Non-Hodgkin's Lymphoma is a CD20 positive B cell NHL, with CD20 expression confirmed by flow cytometry or immunohistochemistry. In further embodiments, the NHL is characterized by at least one of the following features: (a) relapsed/refractory disease that has failed conventional therapy, (b) relapsed disease at least 60 days following autologous stem cell transplantation, (c) relapsed disease at least 4 months following allogeneic stem cell transplantation with no evidence of active graft versus host disease, (d) received at least 2 types of prior therapy, at least one being chemotherapy and at least one containing an anti-CD20 monoclonal antibody, (e) measurable disease as defined by Lugano response criteria (for splenomegaly, vertical length of spleen greater than 13 cm; for measurable adenopathy, nodal long-axis diameter greater than 1.5 cm (a unidimensional measurement)).

In some embodiments, the subject in need thereof has NHL characterized by more than one of the criteria described herein. In specific embodiments, the subject or patient is suffering from NHL characterized by:
(a) relapsed/refractory disease that has failed conventional therapy;
(b) received at least 2 prior lines of therapy (at least one of which contained chemotherapy and at least one of which contained an anti-CD20 monoclonal antibody);
(c) measurable disease as defined by the Lugano response criteria (Cheson et al. 2014, J. Clin Oncol 32(27):3059-3067);
(d) for FL transformed to HGBCL, must have received at least one line of therapy after transformation to HGBCL

In some embodiments, a subject in need thereof can be further defined according to the following criteria: a performance score of at least 60% by Karnofsky, and adequate organ function defined as: a. Cardiac function: Left ventricular ejection fraction (LVEF) of ≥40% by echocardiogram, radionuclide scan or cardiac MRI; b. Pulmonary function: Oxygen saturation at least 90% on room air, pulmonary function tests demonstrating FVC and FEV1 of ≥50% of predicted for age and cDLCO≥50% of predicted; c. Renal function: Creatinine clearance test (by Cockcroft-Gault equation)≥40 mL/min or creatinine≤1.5 mg/dL, d. Hepatic function: Total Serum Bilirubin≤1.5× upper limit of institutional norm, Hepatic transaminases (ALT and AST) <3×upper limit of institutional normal range; e Hematology: Total white blood cell (WBC) count≥3000/μL, absolute neutrophil count (ANC)≥1000/μL, platelet count≥75,000/μL and hemoglobin≥8.0 g/dL (may be waived if abnormalities are due to disease related bone marrow involvement), and f. Calcium (for multiple myeloma patients only): Corrected calcium<11.5 mg/dL within 2 weeks prior to enrollment for treatment.

In some embodiments, eligible subjects should be capable of discontinuing prednisone or other immunosuppressive medications for at least 3 days prior to the cell infusion (excluding preparative regimen pre-medications). Sexually active females of childbearing potential and males with partners of childbearing potential may be requested to agree to use effective contraception during therapy and for 4 months after completion of therapy.

In some embodiments, subjects can be excluded from consideration for treatment for any of the following:
1. High titer of donor specific anti-HLA antibodies (MFI>1000);
2. Active, untreated CNS involvement;
3. Chronic lymphocytic leukemia (CLL)/small lymphocytic lymphoma (SLL), or high-grade lymphomas (Burkitt lymphoma/Lymphoblastic lymphoma);
4. Pregnant or breastfeeding;
5. Marked baseline prolongation of QT/QTc interval (e.g. demonstration of a QTc interval greater than 500 milliseconds);
6. Class II or greater New York Heart Association Functional Classification criteria (appendix III) or serious cardiac arrhythmias likely to increase the risk of cardiac complications of cytokine therapy (e.g. ventricular tachycardia, frequent ventricular ectopy, or supraventricular tachyarrhythmia requiring chronic therapy);
7. Active autoimmune disease requiring immunosuppressive therapy;
8. History of severe asthma, presently on chronic medications (a history of mild asthma requiring inhaled steroids only is eligible);
9. New or progressive pulmonary infiltrates on screening chest x-ray or chest CT scan [unless cleared for study by a pulmonary specialist. Infiltrates attributed to infection must be stable/improving (with associated clinical improvement) after 1 week of appropriate therapy (4 weeks for presumed or documented fungal infections)];
10. Active uncontrolled bacterial, fungal, or viral infections—all prior infections must have resolved following optimal therapy;
11. Known hypersensitivity to any of the therapeutic agents used in the methods of the invention;
12. Received investigational drugs within the 14 days before initiation of treatment with NK cell fraction;

In some embodiments, cell donors (for example, candidates for apheresis) are selected according to the following criteria:
1. 12 to 70 years of age—Priority should be given to age (<35 years), followed by HLA matching (haploidentical and if not available then fully mismatched donor);
2. At least 40 kilogram body weight;
3. In general good health as determined by an evaluating medical provider;
4. Adequate organ function defined as: Hematologic: hemoglobin, WBC, platelet within 10% of upper and lower limit of normal range of test (gender based for hemoglobin), Hepatic: ALT<2× upper limit of normal and Renal: serum creatinine<1.8 mg/dL;
5. Completion of a donor infectious disease screen panel including CMV Antibody, Hepatitis B Surface Antigen, Hepatitis B Core Antibody, Hepatitis C Antibody, HIV PCR, HIV ½ Antibody, HTLVA ½ Antibody, Rapid Plasma (RPR) Treponema, Trypanosoma Cruzi (T. Cruzi), HCV by NAT, HIV by NAT and WNV (West Nile Virus) by NAT or per current panel—must be negative for HIV and active hepatitis B;
6. Not pregnant—females of childbearing potential must have a negative pregnancy test within 7 days of apheresis;
7. Able and willing to undergo apheresis;
8. Voluntary written consent (using assent form if donor<18 years of age).

In some embodiments, the subject in need thereof receives a lymphodepleting preparatory regime. In specific embodiments, the subject is subjected to lymphodepleting preparatory regime prior to transplantation or administration of the compositions of the present invention. The regime can include cyclophosphamide, fludarabine, or other chemotherapy and/or immunosuppressive therapy.

### Combination Therapy

In some embodiments, the subject in need is treated with the expanded CD3-depleted cell fraction described herein in conjunction with additional cancer therapy. In some embodiments, the additional cancer therapy includes a cytotoxic agent and/or non-cytotoxic agent. A “cytotoxic agent” refers to a substance that inhibits or prevents the function of cells and/or causes destruction of cells. The term is intended to include radioactive isotopes (e.g. ¹³¹I, ¹²⁵I, ⁹⁰Y and ¹⁸⁶Re), chemotherapeutic agents, and toxins such as enzymatically active toxins of bacterial, fungal, plant or animal origin or synthetic toxins, or fragments thereof. A non-cytotoxic agent refers to a substance that does not inhibit or prevent the function of cells and/or does not cause destruction of cells. A “non-cytotoxic agent” may include an agent that can be activated to be cytotoxic. A non-cytotoxic agent may include a bead, liposome, matrix or particle (see, e.g., U.S. Patent Publications 2003/0028071 and 2003/0032995, which are incorporated by reference herein). Such agents may be conjugated, coupled, linked or associated with an expanded CD3− depleted NK cell fraction composition described herein.

In some embodiments, conventional cancer medicaments are administered with the compositions described herein. In some cases, the subject in need is treated with the thawed, cryopreserved expanded CD3-depleted cell fraction described herein in conjunction with one or more additional agents directed to target cancer cells. Highly suitable agents include those agents that promote DNA-damage, e.g., double stranded breaks in cellular DNA, in cancer cells. Any form of DNA-damaging agent know to those of skill in the art can be used. DNA damage can typically be produced by radiation therapy and/or chemotherapy. DNA-damaging agents are also referred to as genotoxic agents. As used herein, “in conjunction with” shall mean that the expanded CD3-depleted NK cell fraction is administered to a subject concurrently with one or more additional therapies (either simultaneously or separately but in close proximity), prior to, or after administration of one or more additional therapies.

Examples of radiation therapy include, without limitation, external radiation therapy and internal radiation therapy (also called brachytherapy). Energy sources for external radiation therapy include x-rays, gamma rays and particle beams, energy sources used in internal radiation include radioactive iodine (iodine¹²⁵ or iodine¹³¹), strontium⁸⁹, or radioisotopes of phosphorous, palladium, cesium, indium, phosphate, or cobalt. Methods of administering radiation therapy are well known to those of skill in the art.

Examples of DNA-damaging chemotherapeutic agents that may be particularly useful include, without limitation: Busulfan (Myleran), Carboplatin (Paraplatin), Carmustme (BCNU), Chlorambucil (Leukeran), Cisplatin (Platmol), Cyclophosphamide (Cytoxan, Neosar), Dacarbazme (DTIC-Dome), Ifosfamide (Ifex), Lomustme (CCNU), Mechlorethamme (nitrogen mustard, Mustargen), Melphalan (Alkeran), and Procarbazine (Matulane).

A number of other chemotherapeutic agents may be also used for the method described herein, either alone or in combination. These include: methotrexate, vincristine, adriamycin, cisplatin, non-sugar containing chloroethylnitrosoureas, 5-fluorouracil, mitomycin C, bleomycin, doxorubicin, dacarbazine, taxol, fragyline, Meglamine GLA, valrubicin, carmustaine and poliferposan, MM1270, BAY 12-9566, RAS farnesyl transferase inhibitor, farnesyl transferase inhibitor, MMP, MTA/LY231514, LY264618/Lometexol, Glamolec, CI-994, TNP-470, Hycamtin/Topotecan, PKC412, Valspodar/PSC833, Novantrone/Mitroxantrone, Metaret/Suramin, Batimastat, E7070, BCH-4556, CS-682, 9-AC, AG3340, AG3433, Incel/VX-710, VX-853, ZD0101, ISI641, ODN 698, TA 2516/Marmistat, BB2516/Marmistat, CDP 845, D2163, PD183805, DX8951f, Lemonal DP 2202, FK 317, Picibanil/OK-432, AD 32/Valrubicin, Metastron/strontium derivative, Temodal/Temozolomide, Evacet/liposomal doxorubicin, Yewtaxan/Paclitaxel, Taxol/Paclitaxel, Xeload/Capecitabine, Furtulon/Doxifluridine, Cyclopax/oral paclitaxel, Oral Taxoid, SPU-077/Cisplatin, HMR 1275/Flavopiridol, CP-358 (774)/EGFR, CP-609 (754)/RAS oncogene inhibitor, BMS-182751/oral platinum, UFT(Tegafur/Uracil), Ergamisol/Levamisole, Eniluracil/776C85/5FU enhancer, Campto/Levamisole, Camptosar/Irinotecan, Tumodex/Ralitrexed, Leustatin/Cladribine, Paxex/Paclitaxel, Doxil/liposomal doxorubicin, Caelyx/liposomal doxorubicin, Fludara/Fludarabine, Pharmarubicin/Epirubicin, DepoCyt, ZD1839, LU 79553/Bis-Naphtalimide, LU 103793/Dolastain, Caetyx/liposomal doxorubicin, Gemzar/Gemcitabine, ZD 0473/Anormed, YM 116, iodine seeds, CDK4 and CDK2 inhibitors, PARP inhibitors, D4809/Dexifosamide, Ifes/Mesnex/Ifosamide, Vumon/Teniposide, Paraplatin/Carboplatin, Plantinol/cisplatin, Vepeside/Etoposide, ZD 9331, Taxotere/Docetaxel, prodrug of guanine arabinoside, Taxane Analog, nitrosoureas, alkylating agents such as melphelan and cyclophosphamide, Aminoglutethimide, Asparaginase, Busulfan, Carboplatin, Chlorombucil, cisplatin, Cytarabine HCl, Dactinomycin, Daunorubicin HCl, Estramustine phosphate sodium, Etoposide (VP16-213), Floxuridine, Fluorouracil (5-FU), Flutamide, Hydroxyurea (hydroxycarbamide), Ifosfamide, Interferon Alfa-2a, Alfa-2b, Leuprolide acetate (LHRH-releasing factor analog), Lomustine (CCNU), Mechlorethamine HCl (nitrogen mustard), Mercaptopurine, Mesna, Mitotane (o.p′-DDD), Mitoxantrone HCl, Octreotide, Plicamycin, Procarbazine HCl, Streptozocin, Tamoxifen citrate, Thioguanine, Thiotepa, Vinblastine sulfate, Amsacrine (m-AMSA), Azacitidine, Erthropoietin, Hexamethylmelamine (HMM), Interleukin 2, Mitoguazone (methyl-GAG; methyl glyoxal bis-guanylhydrazone; MGBG), Pentostatin (2′deoxycoformycin), Semustine (methyl-CCNU), Teniposide (VM-26), and Vindesine sulfate, but it is not so limited.

In addition, the following agents may be also useful for the instant invention: alkylating agents, such as carboplatin and cisplatin, nitrogen mustard alkylating agents, nitrosourea alkylating agents, such as carmustine (BCNU), antimetabolites, such as methotrexate, folinic acid, purine analog antimetabolites, mercaptopurine, pyrimidine analog antimetabolites, such as fluorouracil (5-FU) and gemcitabine (Gemzar®), hormonal antineoplastics, such as goserelin, leuprolide, and tamoxifen, natural antineoplastics, such as aldesleukin, interleukin-2, docetaxel, etoposide (VP-16), interferon alfa, paclitaxel (Taxol®), and tretinoin (ATRA), antibiotic natural antineoplastics, such as bleomycin, dactmomycin, daunorubicin, doxorubicin, daunomycin and mitomycins including mitomycin C, and vinca alkaloid natural antineoplastics, such as vinblastine, vincristine, vindesine, hydroxyurea, acetone, adriamycin, ifosfamide, enocitabine, epitiostanol, aclarubicin, ancitabine, nimustine, procarbazine hydrochloride, carboquone, carboplatin, carmofur, chromomycin A3, antitumor polysaccharides, antitumor platelet factors, cyclophosphamide (Cytoxan®), Schizophyllan, cytarabine (cytosine arabinoside), dacarbazine, thiomosine, thiotepa, tegafur, dolastatins, dolastatin analogs such as auristatin, CPT-11 (irinotecan), mitozantrone, vinorelbine, teniposide, aminopterin, carbomycin, esperamicins (See, e.g., U.S. Pat. No. 4,675,187, which is incorporated by reference herein), neocarzinostatin, OK 432, bleomycin, furtulon, broxundine, busulfan, honvan, peplomycin, bestatin (Ubenimex®), interferon-0, mepitiostane, mitobromtol, melphalan, laminin peptides, lentinan, Coriolus versicolor extract, tegafur/uracil, estramustine (estrogen/mechlorethamine), thalidomide, and lenalidomide (Revlmid®).

Other suitable chemotherapeutics include proteasome inhibiting agents. Proteasome inhibitors block the action of proteasomes, cellular complexes that degrade proteins, particularly those short-lived proteins that are involved in cell maintenance, growth, division, and cell death. Examples of proteasome inhibitors include bortezomib (Velcade®), lactacystin (AG Scientific, Inc, San Diego, Calif.), MG132 (Biomol International, Plymouth Meeting, Pa.) PS-519, eponemycin, epoxomycin, aclacinomycin A, the dipeptide benzamide, CVT-63417, and vinyl sulfone tripeptide proteasome inhibitors.

In some embodiments, the methods and cell fractions described herein are used in conjunction with one or more other cancer treatments, including cancer immunotherapy. Cancer immunotherapy is the use of the immune system to reject cancer. The main premise is stimulating the subject's immune system to attack the tumor cells that are responsible for the disease. This can be either through immunization of the subject, in which case the subject's own immune system is rendered to recognize tumor cells as targets to be destroyed, or through the administration of therapeutics, such as antibodies, as drugs, in which case the subject's immune system is recruited to destroy tumor cells by the therapeutic agents. Cancer immunotherapy includes antibody-based therapies and cytokine-based therapies.

The cytokine-based cancer therapy utilizes one or more cytokines that modulate a subject's immune response. Non-limiting examples of cytokines useful in cancer treatment include interferon-alpha (IFN-alpha), interleukin-2 (IL-2), Granulocyte-macrophage colony-stimulating factor (GM-CSF) and interleukin-12 (IL-12).

In order to facilitate tumor targeting and antibody dependent cellular cytotoxicity (ADCC), in some embodiments, disease specific monoclonal antibodies can be administered to the subject in need thereof in conjunction with (e.g. prior to, concomitantly with or following) the expanded CD3-depleted NK cell fraction described herein.

A non-limiting list of monoclonal antibodies suitable for use along with the methods and expanded CD3-depleted NK cell fractions and compositions of the present invention, their cancer cell targets and some of the specific diseases currently indicated for their use is provided in Table 5 below.

TABLE 5
Anti-Cancer Therapeutic Monoclonal Antibodies

| Antibody | Cancer Indication | Target |
|---|---|---|
| Trastuzumab (Herceptin ®) | B/C, gastric, gastrointestinal junction | HER2 |
| | adenocarcinoma | |
| Pertuzumab (Perjeta ®) | HER2+ B/C | HER2 |
| Certuximab (Erbitux ®) | Metastatic CRC, HNSCC | EGFR |
| Panitumumab (Vectibix ®) | Metastatic CRC | EGFR |
| Necitumumab (Portrazza ®) | Metastatic squamous NSCLC | EFGR |
| Dinutuximab (Unituxin ®) | Pediatric neuroblastoma | GD2 |
| Bevacizumab (Avastin ®) | CRC, NSCLC, B/C, Renal Cell Carcinoma | VEGF-A |
| | (RCC), cervical cancer, Glioblastoma, | |
| | Ovarian, Fallopian, primary peritoneal | |
| | cancer | |
| Ramucirumab (Cyramza ®) | Metastatic gastric, gastroesophageal | VEGFR-2 |
| | junction adenocarcinoma | |
| Olaratumab (Lartruvo ®) | Soft tissue sarcoma | PDGFR-alpha |
| Ipilimumab (Yervoy ®) | Metastatic or cutaneous melanoma, | CTLA-4 |
| Nivolumab (Opdivo ®) | Metastatic melanoma, squamous NSCLC, | PD-1 |
| | NSCLC, RCC, HNSCC | |
| Pembrolizumab | Metastatic melanoma, NSCLC, HNSCC | PD-1 |
| (Keytruda ®) | | |
| Atezolizumab (Tecentriq ®) | Urothelial carcinoma | PD-L1 |
| Ado-trastuzumab emtansine | HER2+ B/C | HER2 |
| (Kadcycla ®) fusion | | |
| Denosumab (Xgeva ®) | Bone metastases | RANKL |
| Alemtuzumab (Campath ®) | CLL | CD52 |
| Avelumab (Bavencio ®) | Merkel cell carcinoma | PD-L1 |
| Blinatumomab (Blincyto ®) | ALL | CD19 |
| Brentuximab vedotin | Hodgkins lymphoma | CD30 |
| (Adcetris ®) | | |
| Capromab pendetide | Prostate | PSMA |
| (ProstaScint ®) | | |
| Daratumumab (Darzalex ®) | Multiple myeloma | CD38 |
| Durvalumab (Imfinzi ®) | Urothelial carcinoma | PD-L1 |
| Elotuzumab (Empliciti ®) | Multiple myeloma | SLAMF7 |
| Ibritumomab tiuxetan | Non-Hodgkins lymphoma | CD20 |
| (Zevalin ®) | | |
| Obinutuzumab (Gazyva ®) | CLL | CD20 |
| Ofatumumab (Arzerra ®) | CLL | CD20 |
| Pertuzumab (Perjeta ®) | Metastatic B/C | HER2 |
| Rituximab (Rituxan ®) | B cell Non-Hodgkins lymphoma | CD20 |
| Rituximab-hyaluronidase | CLL, B-cell lymphoma | CD20 |
| (Rituxan Hycela ®) | | |
| Inotuzumab ozogamicin | ALL | CD22 |
| (Besponsa ®) | | |
| Bevacizumab-awwb | CRC, NSCLC, Glioblastoma, RCC, | VEGF |
| (Mvasi ®) | Cervical cancer | |
| Trastuzumab dkst | B/C, gastric, gastroesophageal | HER2 |
| (Ogivri ®) | | |
| Tositumomab (Bexxar ®) | NHL | |

RCC: Renal Cell Carcinoma; ALL: Acute Lymphoblastic Leukemia; CLL: Chronic Lymphocytic
Leukemia; NSCLC: Non-Small Cell Lung Cancer; HNSCC: Head and neck squamous cell
carcinoma; B/C: Breast Cancer; CRC: Colorectal cancer, NHL: Non-Hodgkin's Lymphoma.

In order to facilitate tumor targeting and antibody dependent cellular cytotoxicity (ADCC), in some embodiments, disease specific monoclonal antibodies can be administered to the subject in need thereof. Wherein the hematological malignancy is NHL, one or more NHL-specific monoclonal antibodies (such as rituximab) is administered to the subject in need thereof. An exemplary dosage of rituximab useful for the method of the invention is 375 mg/m² of the subject (patient). In specific embodiments, disease-specific monoclonal antibody treatment comprises administration of the monoclonal antibody(s) in three doses: first dose 10 days prior to administration (infusion, transplantation) of the NK cell fraction, second dose three days prior to administration (infusion, transplantation) of the NK cell fraction and third, and last dose 11 days following administration (infusion, transplantation) of the NK cell fraction, and in some embodiment, approximately 1 week following administration (infusion, transplantation) of the final (second) NK cell fraction. In certain embodiments, the disease specific monoclonal antibody is administered at 9-11 days before the first dose, at 3 days before the first dose and at 12-16 days following the first dose of the allogenic, thawed cryopreserved expanded CD3− depleted cell fraction.

Standard guidelines for infusion, monitoring reactions and toxicities to monoclonal antibody administration are followed. Elotuzumab is typically administered along with a premedication regimen including dexamethasone, an H1 blocker such as diphenhydramine, an H2 blocker such as ranitidine and acetaminophen prior to start of the infusion.

In some embodiments, the subject in need thereof receives a preparative regime of immunosuppressive therapy prior to administration (infusion, transplantation) of the thawed and diluted CD3-depleted cell fraction of the invention. Suitable immunosuppressive agents include, but are not limited to alkylating agents, purine analogs, antimetabolites, and the like. Some immunosuppressive agents are also considered chemotherapeutic immunosuppressive agent. In specific embodiments, the immunosuppressive therapy comprises administration of cyclophosphamide and fludarabine. An exemplary dosage of cyclophosphamide useful for the method of the invention is 400 mg/m² of the subject (patient), and an exemplary dosage of fludarabine useful for the method of the invention is 25 mg/m² of the subject (patient). In specific embodiments, cyclophosphamide is administered (by IV) 5 days prior to administration (transplantation, infusion) of thawed and diluted CD3-depleted cell fraction of the invention, and the fludarabine is administered (IV) on each one of days 5, 4 and 3 prior to administration (transplantation, infusion) of the thawed and diluted CD3-depleted cell fraction of the invention. Alternatively, fludarabine and cyclophosphamide administration can be adjusted such that the last dose of the immunosuppressive agent is completed 2 or 3 days prior to initiation of the cell fraction administration.

According to the methods of the present invention, in some embodiments, the thawed and diluted CD3-depleted cell fraction of the invention is administered into the subject in need thereof in a single dose, or multiple doses. In specific embodiments, administering the thawed and diluted CD3-depleted cell fraction of the invention comprises administering a single dose of the thawed and diluted CD3-depleted cell fraction of the invention. As used herein, the “single dose” of the thawed and diluted CD3-depleted cell fraction of the invention refers to administration during a single treatment visit, and it will be appreciated that the “single dose” can comprise infusion of the contents of multiple thawed and diluted, cryopreserved expanded CD3-depleted cell cryopreservation bags, depending on the number of cells (i.e. CD56+/CD3−) in each bag and according to the individual parameters (mass, surface area) of each individual patient/subject.

In some embodiments, the total dose of the thawed and diluted CD3-depleted cell fraction of the invention for administration to the subject (patient) comprises between 1×10⁷/cells per kg of the subject and 5×10⁸/cells per kg of the subject, between 2×10⁷/cells per kg of the subject and 2×10⁸/cells per kg of the subject, between 5×10⁷ and 2×10⁸/cells per kg of the subject, or between 2×10⁷ and 5×10⁷/cells per kg of the subject expanded allogeneic thawed NK cells. In some embodiments, the total dose of the thawed and diluted CD3-depleted cell fraction of the invention for administration to the subject (patient) comprises 1×10⁷, 5×10⁷, 1×10⁸ or 2×10⁸ expanded allogeneic thawed NK cells per kg of the subject. In specific embodiments, the total dose of the thawed and diluted CD3-depleted cell fraction of the invention for administration to the subject (patient) comprises 2×10⁸ expanded allogeneic thawed cryopreserved NK cells per kg of the subject.

In some embodiments, the allogeneic thawed and diluted CD3-depleted cell fraction of the invention is provided in a first and a second dose (e.g. in two separate treatment visits, with at least 12 hours, 24 hours, 36 hours, 48 hours, 72 hours, 1 week, 2 weeks or more between the doses).

In some embodiments, the combined said first and said second doses comprise between 1×10⁷/cells per kg of the subject and 5×10⁸/cells per kg of the subject, between 2×10⁷/cells per kg of the subject and 2×10⁸/cells per kg of the subject, between 5×10⁷ and 2×10⁸/cells per kg of the subject, or between 2×10⁷ and 5×10⁷/cells per kg of the subject expanded allogeneic thawed NK cells. In some embodiments, the combined first and second dose of the thawed and diluted CD3-depleted cell fraction of the invention for administration to the subject (patient) comprises 1×10⁷, 5×10⁷, 1×10⁸ or 2×10⁸ expanded allogeneic thawed NK cells per kg of the subject. In specific embodiments, the combined first and second dose of the thawed and diluted CD3-depleted cell fraction of the invention for administration to the subject (patient) comprises 2×10⁸ expanded allogeneic thawed cryopreserved NK cells per kg of the subject.

Administration of thawed and diluted CD3-depleted cell fraction of the invention is may be performed as an inpatient or outpatient procedure. Administration of the CD3-depleted NK cell fractions described herein is by infusion, and in specific embodiments, thawed and diluted CD3-depleted cell fraction of the invention are infused into the subject (patient) within 1 hour of thawing and dilution of the cryopreserved CD3-depleted cell fraction and no later than 4 hours after final product release of the thawed and diluted expanded CD3-depleted cell fraction. In specific embodiments, the thawed and diluted CD3-depleted cell fraction of the invention is maintained, until administration, at room temperature, and is not refrigerated before use.

Thus, in some embodiments, the thawed and diluted allogenic expanded CD3-depleted cell fraction of the invention is administered to the subject no more than 1 hour later after provision for transplantation and no more than 4 hours following final product release of the thawed and diluted cell fraction. In some embodiments, the thawed and diluted expanded CD3− depleted allogenic cell fraction is administered to the subject by intravenous infusion, without a filter or pump, for a duration of no less than 15 and no more than 60 minutes per infusion. In specific embodiments, the cell fraction is administered at a rate not greater than 10 cc/kg weight of the patient/hr.

In some embodiments, the thawed and diluted expanded CD3-depleted allogenic cell fraction is administered to the subject by intravenous infusion, without a pump, with a filter, for a duration of no less than 15 and no more than 60 minutes per infusion. In specific embodiments, the cell fraction is administered at a rate not greater than 10 cc/kg weight of the patient/hr.

In some embodiments, the subject in need thereof receives a supportive regimen of interleukin 2 (IL-2) following the thawed and diluted cell fraction administration.

In some embodiments, IL-2 is administered subcutaneously (SC) at a dosage of 6 MU (for patients weighing<45 kilograms, the IL-2 dosage is 3 MU/m²) on the day of administration of the thawed and diluted CD3-depleted cell fraction of the invention, 2 days later and 4 days after the day of administration. In some embodiments, the IL-2 is administered no sooner than 4 hours after the thawed and diluted CD3-depleted cell fraction of the invention on days of the cell infusion. In certain embodiments, the first two IL-2 doses are administered as part of the hospitalization for the cell infusion. The third IL-2 dosage may be administered in an outpatient context. Thus, in specific embodiments, I1-2 administration comprises administering 6×10⁶ units IL-2 following transfusion of expanded CD3-depleted NK cells:
(i) on the day of transfusion of said thawed and diluted expanded allogeneic CD3-depleted cell fraction of the invention, and
(ii) two days following transfusion of said thawed and diluted expanded allogenic CD3− depleted cells, and
(iii) four days following transfusion of said thawed and diluted expanded allogenic CD3− depleted cells.

To reduce the intensity of fever and chills, premedication with acetaminophen/paracetamol 500-1000 mg PO and diphenhydramine 25-50 mg PO/IV before and 4 hours after each dose of IL-2 is recommended.

Further, if the patient has experienced grade 3 toxicity, and the toxicity resolves to grade 2 or better within 48 hours, IL-2 can be resumed at a reduced dosage (for example, 2-4 million units per m squared). If it worsens, IL-2 should be discontinued. If patient has experienced grade 2 or greater IL-2 infusion-related toxicity with the first or second dose, the dose of IL-2 may be held for up to 48 hours. If the toxicity resolves to grade 1 or better within the 48 hours, IL-2 may be given with all planned doses to be given; however the administration of remaining dose(s) is to be at least 24 hours apart.

In some embodiments, subjects can receive any or all of the following: infusion support (e.g. diphenhydramine or dexchlorpheniramine, hydrocortisone and acetaminophen), supportive cytokines (e.g. G-CSF), blood products as needed, anti-viral, anti-bacterial, PCP and/or fungal prophylaxis, CMV, EBV and HHV6 surveillance and IV immunoglobulin as needed.

In some embodiments, subjects receive any or all of an additional treatment for the hematological disease. Said treatment can be a treatment selected from the group consisting of an immunosuppressive treatment, chemotherapy and radio-therapy.

Thus, in some embodiments there is provided a method of treating a hematological disease in a subject in need thereof, the method comprising:
(i) obtaining an apheresis product comprising NK cells and CD3+ cells allogeneic to the subject;
(ii) separating the apheresis product into a CD3-depleted cell fraction and a CD3+ cell fraction;
(iii) inactivating cells of said CD3+ cell fraction by irradiation;
(iv) ex vivo culturing said CD3-depleted cell fraction with the inactivated, irradiated CD3+ cell fraction under conditions allowing for cell proliferation, wherein said conditions comprise providing nutrients, serum, IL-15, CD3 agonist and nicotinamide in an amount between 1.0 mM to 10 mM;
(v) supplementing said combined CD3-depleted and CD3+ cell fractions with fresh nutrients, serum, IL-15 and nicotinamide 6-10 days following step (d) to produce an expanded CD3-depleted cell fraction;
(vi) harvesting said combined CD3-depleted and CD3+ cell fractions 14-16 days following step (d); and
(vii) washing and concentrating said combined CD3-depleted and CD3+ cell fraction of step (vi),
(viii) suspending the washed and concentrated cells of (vii) in a DMSO-free cryopreservation buffer;
(ix) adding DMSO to 10% v/v
(x) reducing temperature of the cells to −120° C., and
(xi) storing the cryopreserved cells at <−120° C.
thereby producing a cryopreserved expanded CD3-depleted cell fraction;
(xii) thawing the cryopreserved cell fraction of (xi) in a 37° C. water bath,
(xiii) diluting the thawed cell fraction of (xii) with an infusion solution;
thereby producing a thawed, diluted cryopreserved NK cell fraction for transplantation;
(xiv) administering an anti-cancer monoclonal antibody to the subject;
(xv) administering at least one immunosuppressive agent to the subject;
(xvi) administering the thawed, diluted cryopreserved NK cell fraction (xiii) into the subject in need thereof; and
(xix) administering IL-2 to the subject,
thereby treating the hematological disease in the subject.

In some specific embodiments, transplantation (administration, infusion) of the thawed and diluted CD3-depleted cell fraction of the invention is preceded by a safety assessment of the subject in need thereof on the day of cell transplantation, typically including a physical examination, CBC, blood chemistry (e.g. at least serum creatinine, total bilirubin, alkaline phosphatase, AST, ALT and magnesium), Vital Signs: weight, temperature, blood pressure, pulse, and respiratory rate, and administration of concomitant medication, including RBC and platelet transfusions.

Infusion of the thawed and diluted CD3-depleted cell fraction of the invention into the subject in need thereof is typically done by infusion via the patient's central venous catheter, subject to the limitations of individual site practice.

The method of treatment of hematological disease of the present invention can be used to treat hematological malignancies, including, but not limited to MM and NHL. As used herein, the term “treating a hematological disease” or “treating a hematological malignancy” refers to reducing the symptoms or signs of the hematological disease. In some embodiments, treating hematological diseases or a hematological malignancy is assessed according to, but not exclusively, reduction in symptoms over time, improvement in clinical parameters, reduced hospitalization and reduced risk of relapse or mortality.

In some embodiments, infusion of thawed and diluted CD3-depleted cell fraction of the invention described herein increases the probability of successful in-vivo expansion of the infused NK cells when compared to infusion of NK cells not cultured and/or administered according to the methods described herein. In some embodiments, the success of expansion in-vivo is measured on days 7 and 14 following infusion.

In other embodiments, infusion of thawed and diluted CD3-depleted cell fraction of the invention described herein increases the function of the NK cells in the peripheral blood of the subject when compared to infusion of NK cells not cultured and/or administered according to the methods described herein. In some embodiments, NK cell function is measured on days 7 and 14 following infusion.

According to some embodiments of the method of the present invention, infusion of thawed and diluted CD3-depleted cell fraction of the invention described herein increases the probability of favorable disease response following infusion of the NK cell fraction, when compared to infusion of NK cells not cultured and/or administered according to the methods described herein. In some embodiments, NK cell function is measured on day 28 and at one year following infusion. In specific embodiments, the hematological malignancy is NHL and the disease response criteria for NHL are assessed according to the Lugano or International Working Group Response Criteria for NHL (for details, see Cheson, et al, J Clin Oncol 2014; 32:3059-68).

In some embodiments, the article of manufacture, composition or kit of the present invention further comprises instructions for administering the expanded NK cell fractions suitable for transplantation into a subject in need thereof.

In some embodiments of the article of manufacture, composition or kit of the present invention, the thawed and diluted CD3-depleted cell fraction of the invention suitable for transplantation into a subject in need thereof comprises at least 7×10⁸ total viable NK cells. In some embodiments, the thawed and diluted expanded cell fractions suitable for transplantation into a subject in need thereof comprises at least 8×10⁸ total viable NK cells, at least 10×10⁸ total viable NK cells, at least 15×10⁸ total viable NK cells, at least 20×10⁸ total viable NK cells, at least 25×10⁸ total viable NK cells, at least 40×10⁸ total viable NK cells, at least 50×10⁸ total viable NK cells, at least 60×10⁸ total viable NK cells, at least 80×10⁸ total viable NK cells or at least 100×10⁸ total viable NK cells. In some embodiments, each cryopreservation bag of the composition comprises in the range of 40-60×10⁸ total viable NK cells. In particular embodiments, each cryopreservation bag comprises 50×10⁸ total viable NK cells. In some embodiments, the composition, article of manufacture or kit comprises up to 4 cryopreservation bags with at least 50×10⁸ total viable NK cells each.

Selected cell populations of the present invention can be provided per se, along with the culture medium containing same, isolated from the culture medium, and combined with a pharmaceutically acceptable carrier as well as with additional agents which may promote cell engraftment and/or organ function (e.g., immunosuppressing agents, antibiotics, growth factor). Hence, cell populations of the invention can be administered in a pharmaceutically acceptable carrier or diluent, such as sterile saline and aqueous buffer solutions. The use of such carriers and diluents is well known in the art.

Compositions of the present invention may, if desired, be presented in a pack or dispenser device, such as an FDA-approved kit or article of manufacture, which may contain one or more unit dosage forms containing the active ingredient (e.g., cells). The pack may, for example, comprise metal or plastic foil, such as a blister pack. The pack or dispenser device may be accompanied by instructions for administration. The pack or dispenser device may also be accompanied by a notice in a form prescribed by a governmental agency regulating the manufacture, use, or sale of pharmaceuticals, which notice is reflective of approval by the agency of the form of the compositions for human or veterinary administration. Such notice, for example, may include labeling approved by the U.S. Food and Drug Administration for prescription drugs or of an approved product insert. Compositions comprising a preparation of the invention formulated in a pharmaceutically acceptable carrier may also be prepared, placed in an appropriate container, and labeled for treatment of an indicated condition, as further detailed above.

The cells prepared according to the methods of the present invention can be administered to the subject per se, or in a pharmaceutical composition where it is mixed with suitable carriers or excipients.

As used herein, a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Hereinafter, the phrases “physiologically acceptable carrier” and “pharmaceutically acceptable carrier,” which may be used interchangeably, refer to a carrier or a diluent that does not cause significant irritation to an organism and does not abrogate the biological activity and properties of the administered compound. An adjuvant is included under these phrases.

Herein, the term “excipient” refers to an inert substance added to a pharmaceutical composition to further facilitate administration of an active ingredient. Techniques for formulation and administration of drugs may be found in the latest edition of “Remington's Pharmaceutical Sciences,” Mack Publishing Co., Easton, PA, which is herein fully incorporated by reference.

Pharmaceutical compositions for use in accordance with the present invention thus may be formulated in conventional manner using one or more physiologically acceptable carriers comprising excipients and auxiliaries, which facilitate processing of the active ingredients into preparations that can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer.

Pharmaceutical compositions suitable for use in the context of the present invention include compositions wherein the active ingredients are contained in an amount effective to achieve the intended purpose. More specifically, a “therapeutically effective amount” means an amount of active ingredients (e.g. expanded CD3-depleted NK cells) effective to prevent, alleviate, or ameliorate symptoms of a disorder (e.g., leukemia, multiple myeloma) or prolong the survival of the subject being treated.

Determination of a therapeutically effective amount is well within the capability of those skilled in the art, especially in light of the detailed disclosure provided herein.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration, and dosage can be chosen by the individual physician in view of the patient's condition. (See, e.g., Fingl, E. et al. (1975), “The Pharmacological Basis of Therapeutics,” Ch. 1, p. 1.)

Depending on the severity and responsiveness of the condition to be treated, dosing can be of a single or a plurality of administrations. The amount of a composition to be administered will, of course, be dependent on the subject being treated, the severity of the affliction, the manner of administration, the judgment of the prescribing physician, etc.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXEMPLARY EMBODIMENTS

Embodiment 1. A method of preparing an NK cell fraction for a subject in need thereof, the method comprising:
(a) obtaining an apheresis product comprising NK cells and CD3+ cells allogeneic to subject;
(b) separating the apheresis product into a CD3-depleted cell fraction and a CD3+ cell fraction;
(c) inactivating cells of said CD3+ cell fraction by irradiation;
(d) ex vivo culturing said CD3-depleted cell fraction with the inactivated, irradiated CD3+ cell fraction under conditions allowing for cell proliferation, wherein said conditions comprise providing nutrients, serum, IL-15, a CD3 agonist and nicotinamide in an amount between 1.0 mM to 10 mM;
(e) supplementing said combined CD3-depleted and CD3+ cell fractions with fresh nutrients, serum, IL-15 and nicotinamide 6-10 days following step (d) to produce an expanded CD3-depleted cell fraction;
(f) harvesting said combined CD3-depleted and CD3+ cell fractions 14-16 days following step (d); and
(g) washing and concentrating said combined CD3-depleted and CD3+ cell fraction of step (f),
thereby producing an expanded NK cell fraction for transplantation in said subject.

Embodiment 2. The method of embodiment 1, wherein said apheresis product is a human apheresis product comprising human NK and CD3+ cell fractions.

Embodiment 3. The method of embodiment 1, wherein said serum is human serum.

Embodiment 4. The method of embodiment 3, wherein said conditions for allowing for cell proliferation comprise providing 10% human serum.

Embodiment 5. The method of embodiment 1, wherein said IL-15 comprises 20 ng/ml IL-15.

Embodiment 6. The method of embodiment 1, wherein said CD3 agonist is OKT3.

Embodiment 7. The method of embodiment 6, wherein said OKT3 comprises 1 μg/ml OKT3.

Embodiment 8. The method of embodiment 1, wherein said nicotinamide comprises 7.0 mM nicotinamide.

Embodiment 9. The method of embodiment 1, wherein said nutrients comprise minimal essential cell culture medium.

Embodiment 10. The method of embodiment 1, wherein said NK cells of said expanded NK cell fraction comprise at least 40-97% CD56+/CD3− cells.

Embodiment 11. The method of embodiment 1, wherein said washed and concentrated expanded NK cell fraction generated by step (e) is characterized by the following parameters:
(a) at least 70% CD56+/CD3− cells;
(b) at least 70% viability;
(c) fewer than 1×106 CD3+/CD56− cells/Kg mass of patient, upon infusion;
(d) no more than 5 EU endotoxin/Kg mass of patient, upon infusion;
(e) no mycoplasma, and
(f) sterile.

Embodiment 12. The method of embodiment 11, wherein said CD3+ cells are fewer than 0.5× ×106 CD3+/CD56− cells/Kg mass of patient, upon infusion.

Embodiment 13. The method of embodiment 1, wherein said CD3-depleted and said irradiated CD3+ cells are seeded at a 1:1 ratio in step (d).

Embodiment 14. The method of embodiment 13, wherein said culturing of step (d) is affected in flasks at 0.30-0.40×106 CD3-depleted and 0.30-0.40×106 irradiated CD3+ cells/ml.

Embodiment 15. The method of embodiment 13, wherein said culturing of step (d) is affected in flasks at 0.35×106 CD3-depleted and 0.35×106 irradiated CD3+ cells/ml.

Embodiment 16. The method of embodiment 13, wherein said culturing of step (d) is affected in flasks at 400-900×106 CD3-depleted and 400-900×106 irradiated CD3+ cells per flask.

Embodiment 17. The method of embodiment 13, wherein said culturing of step (d) is affected in flasks at 700×106 CD3-depleted and 700×106 irradiated CD3+ cells per flask.

Embodiment 18. The method of embodiment 13, wherein said expanded CD3-depleted cells have increased expression of CD62L compared to CD3-depleted cells expanded under identical conditions having 5 mM nicotinamide and without said CD3+ cell fraction.

Embodiment 19. The method of embodiment 13, wherein said expanded CD3-depleted cells have increased antibody dependent cytotoxicity compared to CD3-depleted cells expanded under identical conditions having 5 mM nicotinamide and without said CD3+ cell fraction.

Embodiment 20. A method for cryopreservation of NK cell fractions comprising:
(a) suspending NK cells of an NK cell fraction in a DMSO-free cryopreservation buffer;
(b) adding DMSO to 10% v/v,
(c) reducing temperature of the cells to −120° C., and
(d) storing the cryopreserved NK cell at <−120° C.

Embodiment 21. An NK cell fraction prepared according to embodiment 1.

Embodiment 22. A cryopreserved NK cell fraction prepared according to embodiment 20.

Embodiment 23. The cryopreserved NK cell fraction of embodiment 22, provided in a cryopreservation bag comprising 2.5×108 total cells/ml in a volume of 20 ml.

Embodiment 24. A method for preparing a cryopreserved NK cell fraction for administration comprising
(a) thawing the cryopreserved NK cell fraction in a 37° C. water bath;
(b) diluting said cryopreserved NK cell fraction with infusion solution
thereby producing a thawed, cryopreserved NK cell fraction for transplantation.

Embodiment 25. A thawed, cryopreserved NK cell fraction for transplantation produced by the method of embodiment 24, wherein said cryopreserved NK cell fraction is the cryopreserved NK cell fraction of embodiment 23.

Embodiment 26. The thawed, cryopreserved NK cell fraction of embodiment 25, wherein said expanded CD3-depleted cells have increased in-vivo retention in the spleen and bone marrow following infusion into irradiated mice compared to CD3-depleted cells expanded under identical conditions having 5 mM nicotinamide and without said CD3+ cell fraction.

Embodiment 27. The thawed, cryopreserved NK cell fraction for transplantation of embodiment 25, characterized by the following parameters:
(a) at least 70% CD56+/CD3− cells;
(b) at least 70% viability;
(c) fewer than 1×106 CD3+/CD56− cells/Kg mass of patient, upon infusion;
(d) no more than 5 EU endotoxin/Kg mass of patient, upon infusion;
(e) no mycoplasma, and
(f) sterile.

Embodiment 28. The thawed cryopreserved NK cell fraction of embodiment 25, provided in a fluorinated ethylene propylene (FEP) cryopreservation bag.

Embodiment 29. The thawed cryopreserved NK cell fraction of embodiment 28, comprising 2.5×108 cells/ml.

Embodiment 30. The thawed cryopreserved NK cell fraction of embodiment 28, provided in a volume of 100 ml.

Embodiment 31. A method of treating a hematological disease in a human subject in need thereof, the method comprising:
(a) administering an anti-cancer monoclonal antibody to said subject;
(b) administering at least one immunosuppressive agent to said subject;
(c) transplanting an allogeneic thawed, cryopreserved expanded NK cell fraction into said subject in need thereof, wherein said allogeneic thawed cryopreserved expanded NK cell fraction has been expanded by ex-vivo culturing with CD3+ cells, nutrients, serum, IL-15, a CD3 agonist and nicotinamide in an amount between 1.0 mM to 10 mM; and
(d) administering IL-2 to said subject,
thereby treating said hematologic disease in said subject.

Embodiment 32. The method of embodiment 31, wherein said immunosuppressive agent is a chemotherapeutic immunosuppressive agent and/or irradiation.

Embodiment 33. The method of embodiment 31, wherein said hematological disease is a hematologic malignancy.

Embodiment 34. The method of embodiment 31, wherein said hematological disease is non-Hodgkins lymphoma (NHL).

Embodiment 35. The method of embodiment 34, wherein said hematological disease is follicular lymphoma (FL) or a high-grade B-cell lymphoma (HGBCL).

Embodiment 36. The method of embodiment 34, wherein said NHL is CD20 positive B cell NHL.

Embodiment 37. The method of embodiment 34, wherein said NHL is characterized by at least one of:
(a) relapsed/refractory disease that has failed conventional therapy;
(b) patient having received at least 2 prior lines of therapy (at least one of which contained chemotherapy and at least one of which contained an anti-CD20 monoclonal antibody);
c) measurable disease as defined by the Lugano response criteria;
d) wherein said NLH is FL transformed to HGBCL, must have received at least one line of therapy after transformation to HGBCL.

Embodiment 38. The method of embodiment 33, wherein said hematologic malignancy is NHL and said anticancer monoclonal antibody is rituximab (375 mg/m2).

Embodiment 39. The method of embodiment 31, wherein step (a) is performed three times.

Embodiment 40. The method of embodiment 31, wherein step (c) comprises administering a first dose of said allogeneic thawed cryopreserved expanded cell fraction followed two days later by a second dose of said allogeneic thawed cryopreserved expanded NK cell fraction.

Embodiment 41. The method of embodiment 40, wherein step (a) is performed three times: at 10 days before said first dose, at 3 days before said first dose and at 12-16 days following said first dose of said allogeneic thawed cryopreserved expanded NK cell fraction.

Embodiment 42. The method of embodiment 31, wherein said cell fraction comprises between 1×107 and 5×108 allogeneic thawed cryopreserved expanded NK cells per kg subject.

Embodiment 43. The method of embodiment 31, wherein said cell fraction comprises between 2×108 allogeneic thawed cryopreserved expanded NK cells per kg subject.

Embodiment 44. The method of embodiment 41, wherein the combined said first and said second doses comprise 2×107/kg to 2×108/kg total allogeneic thawed cryopreserved expanded NK cells.

Embodiment 45. The method of embodiment 40, wherein:
(a) said first dose and said second dose of said NK cell fraction each comprise 2.5×107 allogeneic thawed cryopreserved expanded NK cells per kg subject, for a total dose of 5×107 allogeneic thawed cryopreserved expanded NK cells per kg subject, or
(b) said first dose and said second dose of said NK cell fraction each comprise 5×107 allogeneic thawed cryopreserved expanded NK cells per kg subject, for a total dose of 1×108 allogeneic thawed cryopreserved expanded NK cells per kg subject, or
(c) said first dose and said second dose of said NK cell fraction each comprise 1×108 allogeneic thawed cryopreserved expanded NK cells, for a total dose of 2×108/kg allogeneic thawed cryopreserved expanded NK cells per kg subject.

Embodiment 46. The method of embodiment 31, wherein said allogeneic thawed cryopreserved expanded NK cell fraction is administered to said subject no more than 4 hours after thawing.

Embodiment 47. The method of embodiment 31, wherein said allogeneic thawed cryopreserved expanded NK cell fraction is administered to said subject by infusion, without a filter or pump, at a rate not greater than 10 cc/kg weight of the patient/hr.

Embodiment 48. The method of embodiment 31, wherein said at least one immunosuppressive agent comprises cyclophosphamide and/or fludarabine.

Embodiment 49. The method of embodiment 48, wherein:
(i) said at least one immunosuppressive agent comprises both cyclophosphamide (400 mg/m2) and fludarabine (30 mg/m2); and
(ii) wherein said cyclophosphamide and said fludarabine are administered on each one of days 5, 4 and 3 days prior to transfusion of said allogeneic thawed cryopreserved expanded NK cells.

Embodiment 50. The method of embodiment 31, wherein step (d) comprises administering 6×106 units IL-2 following transfusion of said allogeneic thawed cryopreserved expanded NK cells:
(i) on the day of transfusion of said allogeneic thawed cryopreserved expanded NK cells; and
(ii) two days following transfusion of said allogeneic thawed cryopreserved expanded NK cells; and
(iii) four days following transfusion of said allogeneic thawed cryopreserved expanded NK cells.

Embodiment 51. The method of embodiment 31, wherein step (c) comprises transplanting a transplantable NK cell fraction prepared according to the method of embodiment 24.

### EXAMPLES

Reference is now made to the following examples, which together with the above descriptions illustrate some embodiments of the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1, 2, 317, Academic Press; “PCR Protocols: A Guide To Methods And Applications”, Academic Press, San Diego, CA (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1—the Effect of CD3+ Feeder Cells on the Expansion and In Vitro Functionality of Ex Vivo Expanded NK Cells

The effect of autologous irradiated CD3+ cell fraction feeder cells, after inactivation and in the presence of a CD3 agonist such as OKT3, on expansion and function of ex-vivo expanded cryopreserved and thawed NK cells was assessed.

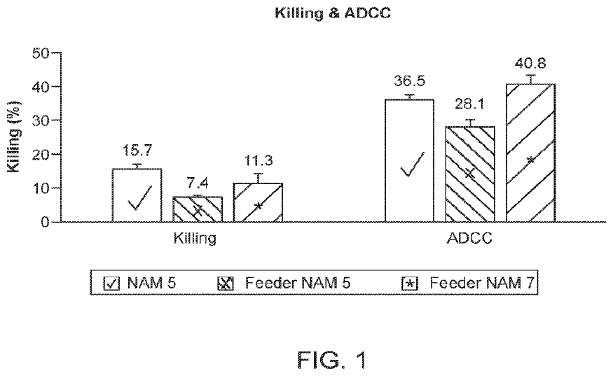

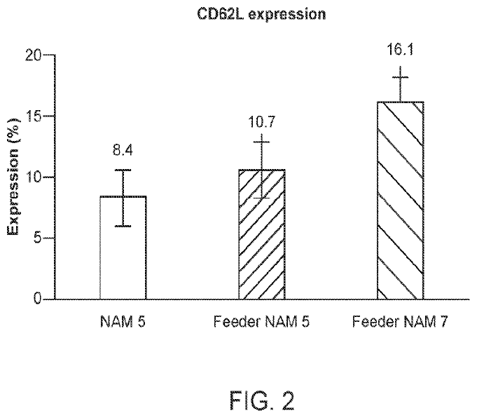

After 2 weeks in culture according to the methods of the invention, a modified procedure using inactivated autologous CD3+ cells as feeder, supplemented with OKT3 and NAM, led to an improved fold expansion, which increased the total number of viable cells obtained at harvest. The functionality of the cells in killing and ADCC was assessed by combination of NK cells with target cells BL2 (Burkitt lymphoma cell line)—at different ratios. Culture of the CD-depleted fraction along with inactivated CD3+ feeder cells led to reduced performance in a cytotoxicity assay which was partially overcome by culturing cells in a higher NAM concentration (7 mM) (FIG. 1). Culturing in 7 mM NAM also led to increased expression of CD62L (FIG. 2).

NK cells play a critical role in surveying and eliminating infected and malignantly transformed cells. They also have the ability to directly kill target cells, as well as mediate ADCC via the membrane receptors FecyRIII (CD16) which binds to the Fc portion of IgG antibody.

To test the influence of NAM on the cytotoxic activity, NK cells were cultured according to the methods of the invention for 2 weeks in 5- or 7-mM NAM with or without feeders. ADCC assay was performed using labeled BL2 cells. Fluorescence-activated cell sorting (FACS) analysis showed increase in cytotoxic activity of NK cells cultured with 7 mM NAM and feeders compared to cells cultured with NAM alone (FIG. 1).

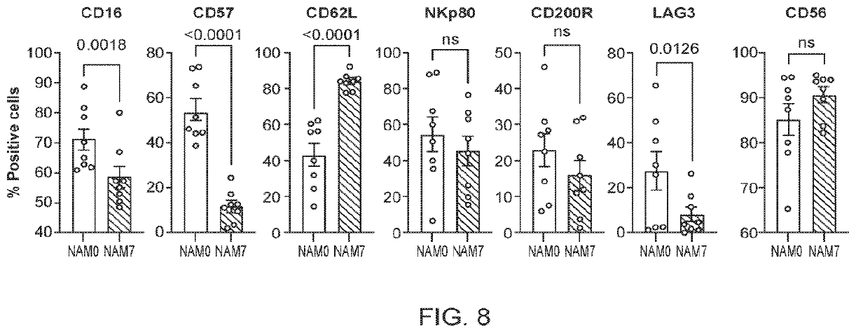

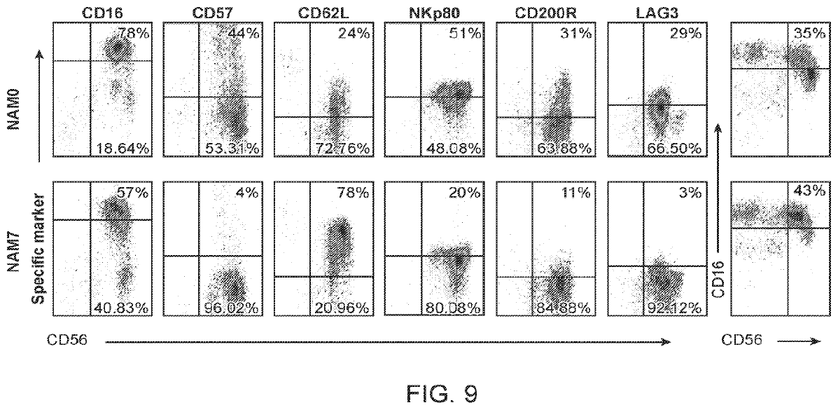

Following culture using the methods of the present disclosure, the phenotypes of cells produced were analyzed using FACS. The results are shown in FIGS. 8 and 9. As shown in FIGS. 8 and 9, the production methods described herein produce cells that have a unique phenotype of surface markers, including a unique expression pattern of CD16, CD57, CD62L, NKp80, CD200R, LAG3 and CD56.

Further analysis of cellular phenotypes, viability and fold expansion of certain cells, was performed using a combination of FACS and CEDEX, on several batches of cells harvested following the culturing methods described herein. The results of these analyses are presented in Tables I, IIa and IIb below.

TABLE I
PHENOTYPE - HARVEST (Day 14 of culture with NAM)

| | | | | | | AVERAGE |
|---|---|---|---|---|---|---|
| Parameter | BATCH 1 | BATCH 2 | BATCH 3 | BATCH 4 | BATCH 5 | of Batches 1-5 |
| Total nucleated cells | 27105 | 41274 | 45315 | 58409 | 31503 | 40721 |
| (TNC) (×10\hat{ }6) | | | | | | |
| TNC fold expansion | 9.8 | 6.6 | 8 | 7 | 11 | 8.48 |
| NK cell fold | 35.2 | 22.7 | 42 | 35 | 23 | 31.6 |
| expansion | | | | | | |
| % viability (cedex) | 99 | 99 | 99 | 99 | 99 | 99 |
| % viability (FACS) | 96 | 96 | 96.3 | 95 | 94.6 | 95.6 |
| % T cell | 1.92 | 2.18 | 0.34 | 2.9 | 0 | 1.47 |
| (CD3⁺/CD56⁻) | | | | | | |
| % NKT | 2.67 | 1.12 | 0.34 | 3.02 | 0.07 | 1.44 |
| (CD3⁺/CD56⁺) | | | | | | |
| % B cells (CD19+) | 0.1 | 0.09 | 0.13 | 0.29 | 0.02 | 0.11 |
| % Monocytes | 0.03 | 0.01 | 0.04 | 0 | 0 | 0.016 |
| (CD14+) | | | | | | |
| % NK cells (CD56⁺) | 94.39 | 96.18 | 98.57 | 92.87 | 94.58 | 95.35 |
| % CD16⁺ | 87.02 | 61.18 | 61.2 | 64.84 | 57.62 | 41.35 |
| % CD62⁺ | 82.54 | 84.71 | 82.36 | 56.31 | 87.84 | 78.75 |

TABLE IIa
PHENOTYPE - HARVEST (Day 14 of culture with NAM)

| Parameter | BATCH 6 | BATCH 7 | BATCH 8 |
|---|---|---|---|
| Total nucleated cells | 34500 | 64810 | 60124 |
| (TNC) | | | |
| (×10\hat{ }6) | | | |
| TNC fold expansion | 10 | 9 | 11 |
| NK cell fold | 40 | 55 | 64 |
| expansion | | | |
| % viability (cedex) | 100 | 99 | 100 |
| % viability (FACS) | 99 | 99 | 99 |
| % T cell | 0.27 | 0.49 | 0.00 |
| (CD3⁺/CD56⁻) | | | |
| % NKT | 0.44 | 1.11 | 0.04 |
| (CD3⁺/CD56⁺) | | | |
| % B cells (CD19⁺) | 0.32 | 0.09 | 0.10 |
| % Monocytes | 0.00 | 0.00 | 0.00 |
| (CD14⁺) | | | |
| % NK cells (CD56⁺) | 98.24 | 98.18 | 99.78 |
| % CD16⁺ | 81.93 | * | 63.24 |
| % CD62⁺ | 82.40 | 74.99 | 95.00 |

TABLE IIb
PHENOTYPE - with or without NAM

| | | | | | CD223 | | | | | |
|---|---|---|---|---|---|---|---|---|---|---|
| | CD200R+ | | NKp80+ | | (LAG3)+ | | CD49a+ | | CD57+ | |
| | NAM | NAM | NAM | NAM | NAM | NAM | NAM | NAM | NAM | NAM |
| | 0 mM | 7 mM | 0 mM | 7 mM | 0 mM | 7 mM | 0 mM | 7 mM | 0 mM | 7 mM |
| Batch A | 14.06 | 11.94 | 35.24 | 26.39 | 40.89 | 16.46 | 70.51 | 82.05 | 46.22 | 11.07 |
| Batch B | 30.71 | 10.92 | 88.87 | 76.56 | 1.23 | 0.57 | 49.1 | 75.35 | 43.88 | 12.66 |
| Batch C | 7.43 | 3.68 | 39.71 | 15.77 | 65.62 | 26.42 | 53.03 | 61.78 | 65.55 | 12.47 |
| Batch D | 6.11 | 1.03 | 50.72 | 19.57 | 26.21 | 4.8 | 58.34 | 69.64 | 44.44 | 3.59 |
| Batch E | 45.92 | 30.82 | 87.75 | 66.33 | 2.82 | 1.92 | 64.75 | 96.02 | 38.8 | 2.05 |
| Batch F | 27.16 | 15.88 | 67.74 | 43.83 | 2.8 | 1.45 | | | 51.19 | 9.71 |
| Batch G | 28.3 | 21.07 | 59.72 | 62.93 | 49.33 | 11.21 | 74.05 | 80.2 | 72.96 | 17.25 |
| Batch H | 22.38 | 31.77 | 6.39 | 52.28 | 30.13 | 3.58 | 47.92 | 44.99 | 73.78 | 24.59 |
| Batch I | 30.92 | 22.9 | 70.74 | 69.95 | 28.25 | 7.56 | 36.29 | 65.25 | 49.43 | 17.54 |
| Batch J | 28.5 | 19.17 | 72.77 | 76.68 | 1.98 | 1.11 | 72.26 | 78.1 | 27.03 | 15.91 |
| Batch K | 37.94 | 31.81 | 74.95 | 71.13 | 24.24 | 19.24 | 60.35 | 52.38 | | |
| Batch L | 32.42 | 19.65 | 73.59 | 58.13 | 23.71 | 8.63 | 55.47 | 68.9 | | |
| Batch M | 21.19 | 13.03 | 48.74 | 27.55 | 43.75 | 26.89 | 77.61 | 79.56 | | |
| Batch N | 21.58 | 13.37 | 53.93 | 58.8 | 14.65 | 5.72 | 62.27 | 68.42 | | |

In addition to cellular phenotypes, the potency of the cell batches described above was measured using the first and second potency assays described herein. The results of these analyses are presented in Tables III and IV below.

TABLE III
POTENCY- HARVEST (Day 14)

| | | | Batch 1 | Batch 2 | Batch 3 | Batch 4 | Batch 5 |
|---|---|---|---|---|---|---|---|
| | | Parameter | Day 14 | Day 14 | Day 14 | Day 14 | Day 14 |
| First Potency Assay | | % Killing K562 | 54.7 | 35.8 | 78.65 | 75.3 | 67.0 |
| (Incucyte 12 hr; NK | | (First potency | | | | | |
| cell fraction # to | | assay, target cells | | | | | |
| target cell ratio = 5:1) | | K562) | | | | | |
| | | (First potency | 11 | 25.3 | 29.23 | 16.6 | 24.4 |
| | | % Killing Raji | | | | | |
| | | assay, target cells | | | | | |
| | | Raji) | | | | | |
| | | % Antibody- | | 51.5 | 28.1 | 35.8 | 30.71 |
| | | dependent | | | | | |
| | | cellular | | | | | |
| | | cytotoxicity | | | | | |
| | | (ADCC) Raji | | | | | |
| | | (First potency | | | | | |
| | | assay, target cells | | | | | |
| | | Raji + rituximab) | | | | | |
| Second | K562 | % CD107a+ | 49.18 | 53.76 | 40.11 | 31.94 | 26.43 |
| Potency | (Second | % INFg+ | ND | 13.54 | 11.73 | 19.83 | 8.98 |
| Assay | potency | % TNFa+ | 38.9 | 29.1 | 30.61 | 21.8 | 21.14 |
| (NK cell | assay, target | | | | | | |
| fraction # | cells K562) | | | | | | |
| to target | RAJI | % CD107a+ | 10.08 | 14.98 | 16.89 | 10.51 | 12.22 |
| cell ratio = | (Second | % INFg+ | ND | 3.59 | 4.86 | 10.15 | 4.25 |
| 1:3) | potency | % TNFa+ | 7.92 | 10.49 | 19.15 | 15.8 | 16.4 |
| | assay, target | | | | | | |
| | cells Raji) | | | | | | |
| | RAJI + | % CD107a+ | 24.15 | 25.76 | 24.7 | 14.39 | 24.64 |
| | RTX | % INFg+ | ND | 7.76 | 9.43 | 14.9 | 6.85 |
| | (ADCC) | % TNFa+ | 17.57 | 18.19 | 27.31 | 18.52 | 25.17 |
| | (First | | | | | | |
| | potency | | | | | | |
| | assay, target | | | | | | |
| | cells Raji + | | | | | | |
| | rituximab) | | | | | | |

TABLE IV
POTENCY- HARVEST (Day 14)

| | | | Batch 6 | Batch 7 | Batch 8 |
|---|---|---|---|---|---|
| | | Parameter | Day 14 | Day 14 | Day 14 |
| First Potency Assay | | % Killing K562 | 84.81 | 87.22 | 78.69 |
| (Incucyte 12 hr; NK | | (First potency | | | |
| cell fraction # to | | assay, target cells | | | |
| target cell ratio = 5:1) | | K562) | | | |
| | | % Killing Raji | 64.10 | 67.28 | 17.06 |
| | | (First potency | | | |
| | | assay, target cells | | | |
| | | Raji) | | | |
| | | dependent | 85.34 | 80.75 | 74.23 |
| | | % Antibody- | | | |
| | | cellular | | | |
| | | cytotoxicity | | | |
| | | (ADCC) Raji | | | |
| | | (First potency | | | |
| | | assay, target cells | | | |
| | | Raji + rituximab) | | | |
| Second | K562 | % CD107a+ | 48.86 | 45.32 | 43.06 |
| Potency | (Second | % INFg+ | 27.66 | 36.43 | 19.35 |
| Assay | potency | % TNFa+ | 54.01 | 52.31 | 40.57 |
| (NK cell | assay, target | | | | |
| fraction # | cells K562) | | | | |
| to target | RAJI | % CD107a+ | 16.81 | 9.02 | 4.13 |
| cell ratio = | (Second | % INFg+ | 5.31 | 7.34 | 1.02 |
| 1:3) | potency | % TNFa+ | 21.45 | 16.64 | 4.86 |
| | assay, target | | | | |
| | cells Raji) | | | | |
| | RAJI + | % CD107a+ | 50.95 | 35.97 | 12.18 |
| | RTX | % INFg+ | 24.30 | 27.67 | 2.92 |
| | (ADCC) | % TNFa+ | 59.26 | 47.57 | 17.54 |
| | (First | | | | |
| | potency | | | | |
| | assay, target | | | | |
| | cells Raji + | | | | |
| | rituximab) | | | | |

Tables 3-5 shows results of an assessment of cell number, viability and phenotype characterization of exemplary cryopreserved, thawed CD3-depleted and irradiated CD3+ cells cultured, frozen and thawed according to the methods described herein. T=0 corresponds to thawing 7±2 days after freezing of the fraction.

TABLE 3
Profile of cryopreserved, thawed expanded CD3-depleted and
CD3+ feeder cells following culture and cryopreservation.

| | | ER#1 | | ER#2 | | GMP#1 | GMP#2 |
|---|---|---|---|---|---|---|---|
| | | Post Thaw | | Post Thaw | | Post Thaw | Post Thaw |
| | | T = 0 | 1 month | T = 0 | 1 month | T = 0 | 1 month |
| | Parameter | (1 bag) | (1 bag) | (1 bag) | (1 bag) | (1 bag) | (1 bag) |
| # of Cells | TNC (×10\hat{ }6) | 5239* | 4771* | 4796* | 4801* | 3420** | 1905** |
| Viability | % viability | 85 | 85 | 73 | 73 | 82 | 89 |
| | (cedex) | | | | | | |
| | % viability | 94 | 85 | 85 | 70 | 93.1 | 91.9 |
| | (FACS) | | | | | | |
| Phenotyping | % T Cell | 3.56 | 1.55 | 1.43 | 2.33 | 0.05 | 1.25 |
| | (CD3⁺/CD56⁻) | | | | | | |
| | % NKT Cell | 3.38 | 3.11 | 3.6 | 1.32 | 0.36 | 7.02 |
| | (CD3⁺/CD56⁺) | | | | | | |
| | % B cells | 0.14 | 0.04 | 0.1 | 0.08 | 0.27 | 0.09 |
| | % Monocytes | 0.04 | 0 | 0.0 | 0.02 | 0 | 0.06 |
| | % NK cells | 91.43 | 65 | 94.72 | 95.06 | 92.99 | 91.28 |
| | (CD56⁺) | | | | | | |
| | % CD16⁺ | 87.74 | 33.52 | 43.69 | 58.35 | 30.21 | 39.18 |
| | % CD62⁺ | 74.38 | 53.91 | 85.40 | 80.06 | 56.69 | 44.35 |

*Total nucleated cells frozen in 1 250 ml bag= 5000 × 10⁶ (in 20 ml volume)
**Total nucleated cells frozen in 1 50 ml bag= 2500 × 10⁶ (in 10 ml volume)

TABLE 4
Profile of cryopreserved, thawed expanded CD3-depleted and
CD3+ feeder cells following culture and cryopreservation.

| | Batch 6 | | | |
|---|---|---|---|---|
| | Post Thaw | | | |
| Parameter | T = 0 | 1 month | 3 months | 6 months |
| Total nucleated cells | 2518 | 2410 | 2179 | 2366 |
| (TNC) | | | | |
| (×10\hat{ }6) | | | | |
| % viability (cedex) | 91 | 93 | 91 | 91 |
| % viability (FACS) | 97 | 97 | 96 | 97 |
| % T cell | 0.12 | 0.06 | 0.23 | 0.19 |
| (CD3⁺/CD56⁻) | | | | |
| % NKT | 0.48 | 0.59 | 0.5 | 0.62 |
| (CD3⁺/CD56⁺) | | | | |
| % B cells (CD19⁺) | 0.38 | 0.21 | 0.64 | 0.41 |
| % Monocytes | 0.00 | 0.00 | 0.00 | 0.00 |
| (CD14⁺) | | | | |
| % NK cells (CD56⁺) | 98.17 | 98.68 | 98.39 | 98.35 |
| % CD16⁺ | 41.14 | 30.02 | 53.65 | 66.38 |
| % CD62⁺ | 31.9 | 37.74 | 56.07 | 61.20 |

TABLE 5
Profile of cryopreserved, thawed expanded CD3-depleted and
CD3+ feeder cells following culture and cryopreservation.

| | Batch 7 | | | Batch 8 | | |
|---|---|---|---|---|---|---|
| | Post Thaw | | | | | |
| Parameter | T = 0 | 1 month | 3 months | T = 0 | 1 month | 3 months |
| Total nucleated cells | 1932 | 1816 | 2256 | 5316 | 4875 | 5453 |
| (TNC) (×10\hat{ }6) | | | | | | |
| % viability (cedex) | 90 | 94 | 88 | 92 | 92 | 90 |
| % viability (FACS) | 96 | 94 | 99 | 93 | 100 | 94 |
| % T cell | 0.22 | 0.33 | 0.48 | 0.01 | 0.01 | 0.01 |
| (CD3⁺/CD56⁻) | | | | | | |
| % NKT | 1.77 | 1.17 | 1.50 | 0.14 | 0.23 | 0.06 |
| (CD3⁺/CD56⁺) | | | | | | |
| % B cells (CD19+) | 0.16 | 0.18 | 0.19 | 0.17 | 0.29 | 0.06 |
| % Monocytes | 0.00 | 0.00 | 0.04 | 0.00 | 0.00 | 0.01 |
| (CD14+) | | | | | | |
| % NK cells (CD56⁺) | 97.85 | 97.75 | 97.75 | 99.59 | 99.54 | 99.82 |
| % CD16⁺ | 62.07 | 39.18 | 53.83 | 20.7 | 36.03 | 61.43 |
| % CD62⁺ | 61.1 | 13.94 | 54.65 | 32.62 | 53.35 | 81.35 |

Tables 6-8 shows results of an assessment of cell function (cell killing and potency, as measured by the first potency assay and the second potency assay described herein, respectively) of exemplary cryopreserved, thawed CD3-depleted and irradiated CD3+ cells cultured according to the methods described herein. T=0 corresponds to thawing 7±2 days after freezing of the fraction.

TABLE 6
Function and Potency of cryopreserved, thawed expanded CD3-depleted
and CD3+ feeder cells following culture and cryopreservation.

| | | | ER#1 | | ER#2 | |
|---|---|---|---|---|---|---|
| | | | Post | Post | Post | Post |
| | | | Thaw | Thaw | Thaw | Thaw |
| | | Parameter | T = 0 | 1 month | T = 0 | 1 month |
| Function | | % Killing K562 | 68.1 | 54.6 | 61.3 | 47.07 |
| (Incucyte 12 hr) | | % Killing Raji | 19.3 | 17.2 | 54.1 | 27.79 |
| | | % ADCC Raji | 60.8 | 60.2 | 64.4 | 50.65 |
| Potency | K562 | % CD107a | 31.05 | 27.39 | 35.6 | 37.2 |
| | | % INFg | 17.19 | 25.67 | 8.21 | 18.99 |
| | | % TNFa | 20.99 | 17.88 | 17.73 | 19.72 |
| | RAJI | % CD107a | 9.35 | 8.11 | 22.24 | 13.37 |
| | | % INFg | 3.14 | 9.86 | 4.81 | 13.25 |
| | | % TNFa | 3.63 | 5.53 | 13.01 | 10.93 |
| | RAJI + | % CD107a | 22.75 | 14.14 | 28.45 | 19.31 |
| | RTX | % INFg | 4.91 | 18.63 | 8.36 | 26.21 |
| | (ADCC) | % TNFa | 6.66 | 10.96 | 17.19 | 16.46 |

TABLE 7
Function and Potency of cryopreserved, thawed expanded CD3-depleted
and CD3+ feeder cells following culture and cryopreservation.

| | | | Batch 6 | | | Batch 7 | | |
|---|---|---|---|---|---|---|---|---|
| | | | Post Thaw | | | | | |
| | | Parameter | T = 0 | 1 month | 3 months | T = 0 | 1 month | 3 months |
| First Potency Assay | | % Killing | 65.36 | 93.60 | 68.96 | 57.35 | 52.32 | 86.99 |
| (Incucyte 12 hr; NK | | K562 (First | | | | | | |
| cell fraction # to | | potency assay, | | | | | | |
| target cell ratio = | | target cells | | | | | | |
| 5:1) | | K562) | | | | | | |
| | | % Killing | 47.74 | 84.15 | 28.15 | 35.28 | 11.76 | 56.87 |
| | | Raji (First | | | | | | |
| | | potency assay, | | | | | | |
| | | target cells | | | | | | |
| | | Raji) | | | | | | |
| | | % Antibody- | 83.52 | 93.74 | 67.88 | 69.38 | 31.66 | 70.36 |
| | | dependent | | | | | | |
| | | cellular | | | | | | |
| | | cytotoxicity | | | | | | |
| | | (ADCC) Raji | | | | | | |
| | | (First potency | | | | | | |
| | | assay, target | | | | | | |
| | | cells Raji + | | | | | | |
| | | rituximab) | | | | | | |
| Second | K562 | % CD107a+ | 25.71 | 33.35 | 18.79 | 27.93 | 46.57 | 33.13 |
| Potency | (Second | % INFg+ | 31.08 | 26.91 | 15.58 | 19.53 | 35.18 | 21.78 |
| Assay | potency | % TNFa+ | 36.23 | 39.2 | 23.54 | 29.04 | 47.75 | 32.37 |
| (NK cell | assay, | | | | | | | |
| fraction | target cells | | | | | | | |
| # to | K562) | | | | | | | |
| target | RAJI | % CD107a+ | 7.73 | NA | 7.62 | 4.68 | 7.54 | 11.09 |
| cell | (Second | % INFg+ | 9.54 | NA | 7.32 | 1.73 | 4.72 | 6.33 |
| ratio = | potency | % TNFa+ | 13.45 | NA | 12.14 | 0 | 9.26 | 15.02 |
| 1:3) | assay, | | | | | | | |
| | target cells | | | | | | | |
| | Raji) | | | | | | | |
| | RAJI + | % CD107a+ | 25.23 | NA | 19.76 | 26.02 | 22.40 | 34.48 |
| | RTX | % INFg+ | 33.89 | NA | 19.27 | 23.27 | 15.92 | 19.62 |
| | (ADCC) | % TNFa+ | 41.95 | NA | 31.77 | 33.08 | 24.81 | 35.18 |
| | (First | | | | | | | |
| | potency | | | | | | | |
| | assay, | | | | | | | |
| | target cells | | | | | | | |
| | Raji + | | | | | | | |
| | rituximab) | | | | | | | |

TABLE 8
Function and Potency of cryopreserved, thawed expanded CD3-depleted
and CD3+ feeder cells following culture and cryopreservation.

| | | | Batch 8 | | |
|---|---|---|---|---|---|
| | | | Post Thaw | | |
| | | Parameter | T = 0 | 1 month | 3.5 months |
| First Potency Assay | | K562 (First | 74.16 | 40.38 | 53.32 |
| (Incucyte 12 hr; NK | | % Killing | | | |
| cell fraction # to | | potency assay, | | | |
| target cell ratio = | | target cells | | | |
| 5:1) | | K562) | | | |
| | | % Killing | 35.16 | 47.81 | 31.49 |
| | | Raji (First | | | |
| | | potency assay, | | | |
| | | target cells | | | |
| | | Raji) | | | |
| | | % Antibody- | 71.16 | 70.44 | 64.99 |
| | | dependent | | | |
| | | cellular | | | |
| | | cytotoxicity | | | |
| | | (ADCC) Raji | | | |
| | | (First potency | | | |
| | | assay, target | | | |
| | | cells Raji + | | | |
| | | rituximab) | | | |
| Second | K562 | % CD107a+ | 21.65 | 37.05 | 33.53 |
| Potency | (Second | % INFg+ | 11.54 | 24.68 | 14.65 |
| Assay | potency | % TNFa+ | 17.14 | 33.19 | 23.94 |
| (NK cell | assay, target | | | | |
| fraction | cells K562) | | | | |
| # to | RAJI | % CD107a+ | 4.61 | 16.28 | 9.19 |
| target | (Second | % INFg+ | 1.58 | 9.12 | 2.89 |
| cell ratio = | potency | % TNFa+ | 3.92 | 16.45 | 8.51 |
| 1:3) | assay, target | | | | |
| | cells Raji) | | | | |
| | RAJI + RTX | % CD107a+ | 24.16 | 39.01 | 32.82 |
| | (ADCC) | % INFg+ | 10.04 | 21.9 | 13.48 |
| | (First | % TNFa+ | 22.6 | 35.25 | 27.80 |
| | potency | | | | |
| | assay, target | | | | |
| | cells Raji + | | | | |
| | rituximab) | | | | |

### Example 2—the Effect of NAM and CD3+ Feeder Cells on the Phenotype Characterization of Ex Vivo Expanded NK Cells

The immune checkpoint receptors, CD200R and Programmed Death receptor-1 (PD-1), are exploited by tumor cells which overexpress their ligands to suppress lymphocytes anti-tumor activity. The effect of expansion with nicotinamide (NAM) on expression of immune checkpoint receptors in NK cells was assessed.

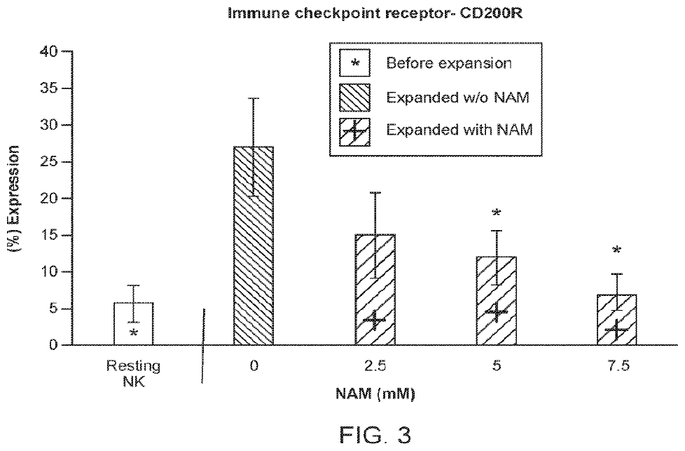

Immunophenotyping using FACS analysis demonstrated that NAM down regulated the expression of CD200R expressed on the expanded NK cells (FIG. 3).

### Example 3 the Effect of NAM and CD3+ Feeder Cells on the In-Vivo Homing of Ex Vivo Expanded NK Cells

During activation in culture, NK cells lose some of their ability to traffic, localize and proliferate in vivo directly impacting their clinical efficacy. The effect of culture with CD3+ feeder cells, cryopreservation and thawing with and without NAM on the retention and retention efficacy of ex vivo expanded NK cells was assessed.

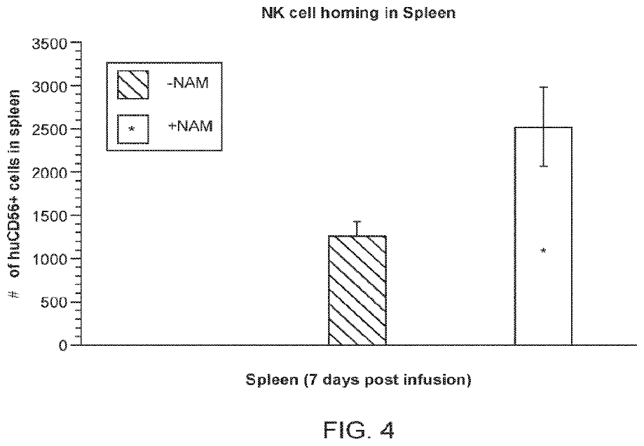

The in vivo retention model (spleen) showed that the overall retention of NK cells from CD3-depleted cultures cultured with NAM and irradiated CD3+ feeder cells, following cryopreservation and thawing according to the methods described herein was higher when compared to the retention of cytokines only cultured cells (FIG. 4).

### Example 4—the Effect of CD3+ Feeder Cells on the In Vivo Functionality of Ex Vivo Expanded NK Cells

The effect of culture with CD3+ feeder cells, and cryopreservation and thawing on NK cell function have been shown in vitro (See Example 1).

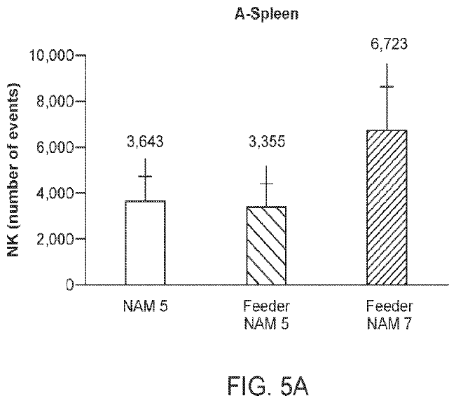

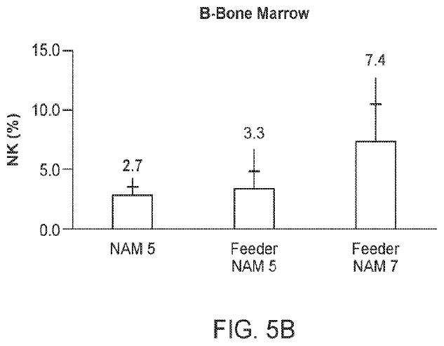

NK expansion with or without feeders in either 5 mM or 7 mM NAM was compared in two different in vivo assays. In a retention assay irradiated NSG mice (n=6-7 mice per group) were injected IV with 20×10⁶ human NK cells/mouse. NK cells expanded with feeders and 7 mM NAM showed increased retention in terms of overall number of NK cells in the spleen (FIG. 5A) and an increased percentage of NK cells in the bone marrow after 3 days (FIG. 5B).

### Example 5—Ex-Vivo Expanded, Cryopreserved and Thawed CD3-Depleted (NK) Cells

The effect of culture with CD3+ feeder cells, and cryopreservation and thawing on NK cell function have been shown in vitro (See Example 1), and in vivo (Example 4). Tumor growth inhibition potential of the expanded NK cells was assessed in A549 (human lung adenocarcinomic epithelial tumor) cells.

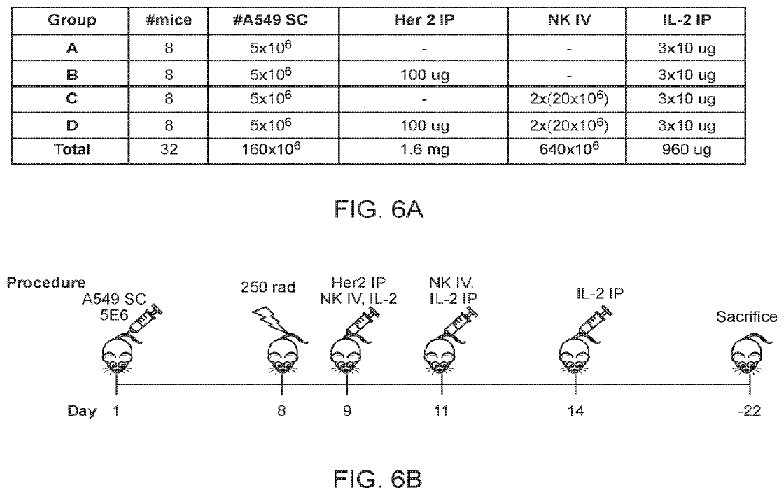

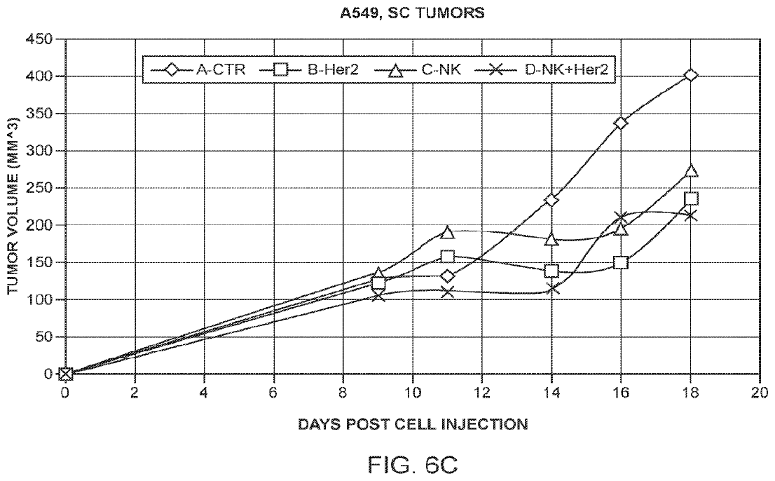

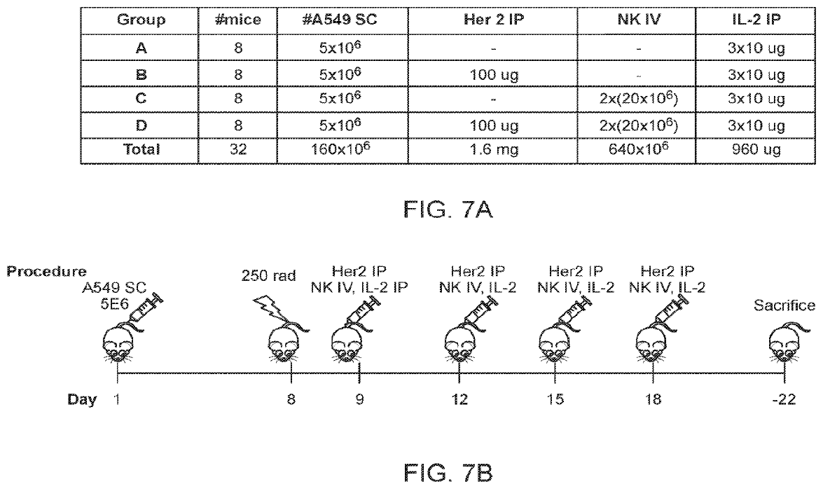

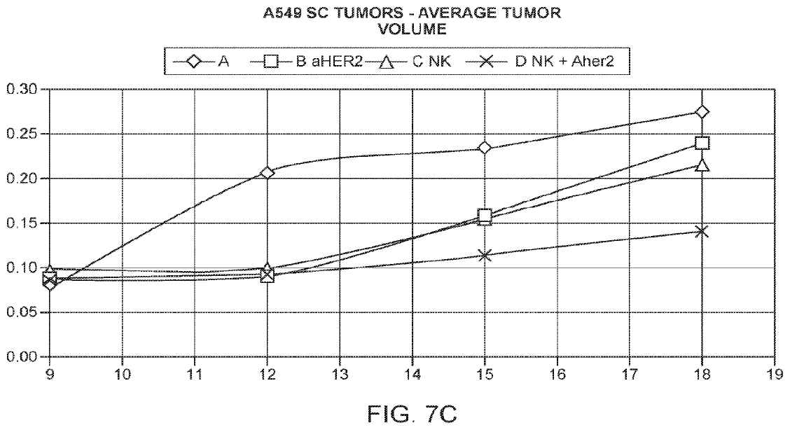

FIGS. 6 and 7 show the synergistic effect of combined NK cells administration with anti-Her2 antibody administration (NK+AHer2), both with minimal NK cell administration (days 9 and 11)(FIGS. 6B and 6C) and multiple, extended NK cell administrations (days 9, 12, 15, and 18) (FIGS. 7B and 7C).

Without wishing to be bound by theory, when taken together, the results presented herein demonstrate that ex-vivo culture of NK cells in the CD3-depleted cell fraction, with nicotinamide, irradiated CD+ feeder cells, and a CD3 agonist (e.g. OKT3) according to the methods described herein, cryopreserved and thawed as described, can provide an effectively storable expanded NK cell population having enhanced ex-vivo and in-vivo functionality, overcoming the negative effects of freezing and thawing and suitable for use in treatment of cancer and other diseases.

1. An NK cell fraction selected comprising a population of nucleated cells,
wherein the population comprises at least 1.0×10⁶ nucleated cells,
wherein at least about 70% of the cells in the population are viable,
wherein:
at least about 85% of the cells in the population are CD56+;
no more than about 0.5% of the cells in the population are CD3+;
no more than about 10% of the cells in the population are CD19+;
no more than about 10% of the cells in the population are CD14+;
no more than about 25% of the cells in the population are CD57+;
at least about 10% of the cells in the population are CD16+; and
at least about 10% of the cells in the population are CD62L+,
said population is sterile and devoid of mycoplasma, and
said population has no more than 0.5 EU/ml endotoxin.

2. The NK cell fraction of claim 1, wherein the target cell death in a K562 cell potency assay is at least about 10%, and the target cell death in Antibody-Dependent Cellular Cytotoxicity (ADCC) Raji cell potency assay is greater than the value for Raji cells without the antibody.

3. The NK cell fraction of claim 1, wherein the percentage of viable cells stained with an anti-CD107a antibody in a degranulation assay is at least 10%.

4.-6. (canceled)

7. The NK cell fraction of claim 1, wherein at least about 15% of the cells in the population are CD62L+.

8. (canceled)

9. The NK cell fraction of claim 1, wherein at least about 20% of the cells in the population are CD16+.

10.-18. (canceled)

19. A cryopreserved NK cell fraction, comprising:
i) the NK cell fraction of claim 1; and
ii) DMSO, wherein the concentration of DMSO is about 10% v/v.

20. The cryopreserved NK cell fraction of claim 19, wherein the cryopreserved NK cell fraction is stable:
i) at about −80° C. for up to six weeks; and/or
ii) at about −150° C. for up to about 12 months.

21. A method of preparing the NK cell fraction of claim 1, the method comprising:
(a) obtaining a human apheresis product comprising NK cells and CD3+ cells allogeneic to subject;
(b) separating the apheresis product into a CD3-depleted cell fraction and a CD3+ cell fraction;
(c) inactivating cells of said CD3+ cell fraction by irradiation;
(d) ex vivo culturing said CD3-depleted cell fraction with the inactivated, irradiated CD3+ cell fraction at a ratio of 1:1 under conditions allowing for cell proliferation, wherein said conditions comprise providing nutrients, serum, IL-15, OKT-3 and nicotinamide in an amount between 1.0 mM to 10 mM, and optionally 7.0 mM;
(e) supplementing said combined CD3-depleted and CD3+ cell fractions with fresh nutrients, serum, IL-15 and nicotinamide 6-10 days following step (d) to produce an expanded CD3-depleted cell fraction;
(f) harvesting said combined CD3-depleted and CD3+ cell fractions 14-16 days following step (d); and
(g) washing and concentrating said combined CD3-depleted and CD3+ cell fraction of step (f),
thereby producing the NK cell fraction.

22.-25. (canceled)

26. The method of claim 21, wherein said culturing of step (d) is affected in flasks at
i) 0.30-0.40×10⁶ CD3-depleted and 0.30-0.40×10⁶ irradiated CD3+ cells/ml;
ii) 0.35×10⁶ CD3-depleted and 0.35×10⁶ irradiated CD3+ cells/ml;
iii) 400-900×10⁶ CD3-depleted and 400-900×10⁶ irradiated CD3+ cells per flask; or
iv) 700×10⁶ CD3-depleted and 700×10⁶ irradiated CD3+ cells per flask.

27. A method for cryopreservation of a NK cell fraction comprising:
(a) suspending the NK cell fraction of claim 1 in a DMSO-free cryopreservation buffer;
(b) adding DMSO to 10% v/v;
(c) reducing temperature of the cells to −120° C.; and
(d) storing the cryopreserved NK cell at a temperature of less than or equal to −120° C.

28. A method for preparing a thawed NK cell fraction comprising:
(a) thawing the cryopreserved NK cell fraction of claim 19 in a 37° C. water bath;
(b) diluting said cryopreserved NK cell fraction thawed in step (a) with infusion solution
thereby producing a thawed, cryopreserved NK cell fraction.

29. A method of treating a hematological disease in a human subject in need thereof, the method comprising:
(a) administering at least one anti-cancer monoclonal antibody to said subject;
(b) administering at least one immunosuppressive agent to said subject;
(c) thawing and administering the NK cell fraction produced by the method of claim 21; and
(d) administering IL-2 to said subject.

30. The method of claim 29, wherein step (c) comprises administering a first dose of said NK cell fraction and two days later administering a second dose of said NK cell fraction.

31. The method of claim 30, wherein:
(i) said first dose and said second dose each comprise at least about 1.25×10⁷ cells/kg, for a total dose of 2.5×10⁷ cells/kg;
(ii) said first dose and said second dose each comprise at least about 2.5×10⁷ cells/kg, for a total dose of at least about 5×10⁷ cells/kg;
(iii) said first dose and said second dose of said NK cell fraction each comprise 5×10⁷ cells/kg, for a total dose of 1×10⁸ cells/kg; or
(iv) said first dose and said second dose of said NK cell fraction each comprise 1×10⁸ cells/kg, for a total dose of 2×10⁸ cells/kg.

32. The method of claim 29, wherein said immunosuppressive agent is a chemotherapeutic immunosuppressive agent, irradiation or any combination thereof.

33. The method of claim 29, wherein said hematological disease is a hematologic malignancy.

34. The method of claim 29, wherein the hematological disease is multiple myeloma.

35. The method of claim 29, wherein said hematological disease is non-Hodgkins lymphoma (NHL), wherein the NHL is:
i) CD20 positive B cell NHL;
ii) follicular lymphoma (FL);
iii) high grade B-cell lymphoma (HGBCL);
iv) HGBCL, not otherwise specified (HGBCL, NOS);
v) primary mediastinal large B-cell lymphoma (PMBCL); or
vi) diffuse large B cell lymphoma (DLBCL), and
wherein said NHL is characterized by at least one of:
a) relapsed/refractory disease that has failed conventional therapy;
b) patient having received at least two prior lines of therapy, preferably wherein at least one of the at least two prior lines of therapy comprised the administration of chemotherapy and at least one of the at least two prior lines of therapy comprise the administration of an anti-CD20 monoclonal antibody;
c) measurable disease as defined by the Lugano response criteria;
d) wherein said NLH is FL transformed to HGBCL, the subject has previously received at least one line of therapy after transformation to HGBCL.

36. (canceled)

37. The method of claim 29, wherein said hematologic disease is NHL and said anticancer monoclonal antibody is rituximab, preferably wherein the rituximab is administered at least once at a dose of about 375 mg/m², preferably wherein rituximab is administered at least three times:
at about 10 days before the first administration of the NK cell fraction or thawed NK cell fraction;
at about 3 days before the first administration of the NK cell fraction or thawed NK cell fraction; and
at 12-16 days following the first administration of the NK cell fraction or thawed NK cell fraction.

38. (canceled)

39. The method of claim 39, wherein said at least one immunosuppressive agent comprises cyclophosphamide and/or fludarabine, or
both cyclophosphamide (400 mg/m²) and fludarabine (30 mg/m²),
wherein the cyclophosphamide is administered at a dose of 400 mg/m² and the fludarabine is administered at a dose of 30 mg/m², and
wherein said cyclophosphamide and said fludarabine are administered:
at about 5 days before the first administration of the NK cell fraction or thawed NK cell fraction;
at about 4 days before the first administration of the NK cell fraction or thawed NK cell fraction; and
at about 4 days before the first administration of the NK cell fraction or thawed NK cell fraction.

40. (canceled)

41. The method of claim 29, wherein step (d) comprises administering 6×10⁶ units IL-2 one each one of following transfusion of said allogeneic thawed cryopreserved expanded NK cells:
(i) on the day of the first administration of the NK cell fraction or thawed NK cell fraction;
(ii) two days following the first administration of the NK cell fraction or thawed NK cell fraction; and
(iii) four days following the first administration of the NK cell fraction or thawed NK cell fraction.

42. A method of treating an autoimmune disease in a human subject in need thereof, the method comprising:
(a) administering at least one anti-cancer monoclonal antibody to said subject;
(b) administering at least one immunosuppressive agent to said subject;
(c) thawing and administering the NK cell fraction produced by the method of claim 12; and
(d) administering IL-2 to said subject.

43. The method of claim 42, wherein step (b) comprises administering a first dose of said NK cell fraction and two days later administering a second dose of said NK cell fraction.

44. A method of treating a solid tumor in a human subject in need thereof, the method comprising:
(a) administering at least one anti-cancer monoclonal antibody to said subject;
(b) administering at least one immunosuppressive agent to said subject;
(c) thawing and administering the NK cell fraction produced by the method of claim 12; and
(d) administering IL-2 to said subject.
